(19) United States
(12) Patent Application Publication
(10) Pub. No.: US 2026/0108537 A1
(43) Pub. Date: Apr. 23, 2026

(54) Integrative Treatment for Shingles Based on Cannabinoid Compounds

(71) Applicant: Cannabis Bioscience International Holdings Inc, Houston, TX (US)

(72) Inventors: Rosangel del Valle Andrades Fuentes, Caracas (VE); Nancy Paola Duarte Delgado, Bogotá (CO); Nancy Rodrigues Das Fontes, Viseu (PT); Stephanie Dona Hartmann, Caracas (VE); Ana Maria Maldonado Vacca, Bogotá (CO); Maria Angelica Clavijo Romero, Santa Marta (CO)

(73) Assignee: Cannabis Bioscience International Holdings Inc, Houston, TX (US)

(21) Appl. No.: 18/920,359

(22) Filed: Oct. 18, 2024

(51) Int. Cl. A61K 31/00; A61K 9/06; A61K 9/107; A61K 9/48; A61K 31/165; A61K 36/886; A61K 47/10; A61K 47/14; A61K 47/22; A61K 47/26; A61K 47/34; A61K 47/38

(52) U.S. Cl. CPC A61K 31/658; A61K 9/06; A61K 9/1075; A61K 9/4858; A61K 31/165; A61K 36/886; A61K 47/10; A61K 47/14; A61K 47/22; A61K 47/26; A61K 47/34; A61K 47/38

(57) ABSTRACT

This disclosure describes a cannabinoid-based nanoplatform for treating shingles, designed to enhance bioavailability and controlled release of active ingredients. The composition includes cannabinoids like cannabidiol (CBD) and cannabigerol (CBG), alongside flavonoids, polyphenols, and alkaloids, in oral and topical formulations. The oral capsule combines cannabinoids with polyphenols and flavonoids to provide systemic anti-inflammatory, antiviral, and neuroprotective effects, particularly for nerve pain and inflammation, including postherpetic neuralgia. The topical cream includes cannabinoids, capsaicin, and aloe vera, offering localized relief from pain, itching, and rash. The method targets both systemic and localized symptoms during the acute phase of shingles, aiming to inhibit varicella-zoster virus replication, disrupt viral assembly, and prevent viral release, while also providing immune modulation and anti-inflammatory benefits. The nanoplatform uses nano-emulsification techniques, producing particles smaller than 200 nm to ensure optimal delivery and therapeutic efficacy, offering comprehensive relief for shingles symptoms.

### FIELD OF THE DISCLOSURE

The technical field of the disclosure relates to medicinal, therapeutic, and healing cannabis and cannabinoids, as well as pharmaceutical cannabis and cannabinoids. The present disclosure describes Cannabinoid-Based Compositions for managing shingles symptoms through several routes of administration. These compositions are designed to alleviate key shingles related issues, targeting symptoms such as pain or tingling, rash, itching, sensitivity to touch, flu-like symptoms and swelling. By combining the therapeutic properties of cannabinoids and enhancing them with analgesic agent, flavonoids and polyphenols, this disclosure presents a comprehensive strategy to improve overall well-being in cases of shingles.

### BACKGROUND INFORMATION

Herpes zoster or shingles (HZ), is a viral disease that results from the reactivation of the varicella-zoster virus (VZV), which remains dormant in the sensory ganglia of the cranial nerves or in the dorsal root ganglia after a previous chickenpox infection (Freer & Pistello, 2018). This condition is more common in older adults and in immunosuppressed individuals, when latent VZV is reactivated. VZV causes the first expression of the disease known as chickenpox, which occurs mainly in childhood. Transmission occurs through aerosolized particles of respiratory secretions or skin lesions of an infected person (Arvin et al., 2010). The main cellular targets of VZV are T lymphocytes, epithelial cells and ganglia (Gershon A et al., 2015). During chickenpox, the virus spreads from the skin and mucous membranes to nerves and sensory ending fibers, and finally settles in cranial or dorsal root ganglia. Here, viral fragments reside within neuronal nuclei or satellite cells, protected from circulating antibodies that persist after primary infection.

The structure that envelops the viral genome or capsid facilitates its latent infection in neurons, where it is transported along the axon from nerve endings to cell bodies in sensory ganglia to establish latency and then years later, viral reactivation occurs. The capsid is subsequently transported from neuronal cell bodies to innervated dermatomes in an anterograde manner, leading to the manifestation of herpes zoster (Shingles) (Perciani et al., 2017). Shingles manifests as pain, tingling, or itching in a dermatome that then develops into fluid-filled swellings surrounded by an erythematous area that appears on the area of skin innervated by the infected nerve fibers. The swellings can appear anywhere on the body and are highly contagious. The affected area is usually sensitive to any stimulus causing pain. The swellings continue to form for the next 3 to 5 days and then crust over to resolve.

HZV poses a significant health concern due to its impact on the general population (Gilden et al., 2010). In the United States, herpes zoster is a well-documented condition with clear epidemiological data. The incidence of HZ varies significantly with age, with older adults and immunocompromised individuals being more susceptible. Likewise, recurrences are most common in patients who are immunosuppressed (Parikh R et al., 2024). Younger healthy individuals experience incidence rates between 1.2 and 3.4 cases per 1,000 people per year, while the risk rises to 3.9 to 11.8 per 1,000 for those over 65. While prevalence rates are relatively consistent across different regions, variations exist due to healthcare access, vaccination rates, and demographic factors influencing reporting and healthcare seeking behavior (Nair et al., 2024).

Approximately one in three individuals with HZ experience complications like neuropathic pain, postherpetic neuralgia (PHN) and other serious neurological and ocular disorders like meningoencephalitis, myelitis, cranial nerve palsies, vasculopathy, keratitis and retinopathy, as well as multiple visceral and gastrointestinal disorders, including ulcers, hepatitis and pancreatitis (Gilden et all, 2010; Parikh R et al, 2024). PHN affects 30% of those afflicted with HZ, underscoring the ongoing impact on quality of life and healthcare resources (Parikh R et al, 2024). Concerning the reduction in the incidence of the virus, the introduction of the shingles vaccine has reduced the incidence and severity of the disease, particularly among older adults. Despite vaccination efforts, there are still limitations to achieving universal coverage and addressing disparities in vaccine acceptance and safety among different populations. The negative effects (Gué, E et al., 2016; Hernández-Cervantes et al., 2017) of vaccination include various possible side effects such as pain, muscle discomfort, redness and swelling at the injection site and headache, fatigue, muscle pain, tremors, fever, and digestive discomfort.

Additionally, the vaccine is preventive and does not resolve the development of the disease or the symptoms present due to the expression of the virus. Hence, shingles vaccination only represents one alternative since it is not always effective and there are safer and more natural options that can help eradicate the virus. Patients with a history of underlying malignancies, steroid or immunosuppressive therapy use, HIV infection, or solid organ transplant are susceptible to disseminated varicella due to impaired cellular immunity, experiencing significant morbidity and elevated mortality rates more often than typical hosts. There is a clear need for a safer and more effective treatment option that addresses the symptoms of HZ without exposing patients to the side effects of antiviral drugs. Therefore, our alternative to conventional treatment is a composition focused primarily on cannabinoids, which target the endocannabinoid system to decrease virus replication and disease worsening, and in turn alleviate associated symptoms such as burning, stabbing pain, fever, erythema, and swellings due to immune system activation.

This composition also incorporates flavonoids, such as quercetin, and polyphenols, like resveratrol, which offer additional therapeutic benefits. These compounds have antioxidant effects and reduce the number of virions per infected cell, which generates an unfavorable environment for viral replication. This combination creates a comprehensive treatment that addresses both virus replication and the symptoms of herpes zoster expression in which cannabinoids CBD (Cannabidiol), CBG (Cannabigerol) act as the main therapeutic component, offering an effective and safer alternative to drug therapy such as antivirals acyclovir (Zovirax), famciclovir or valacyclovir (Valtrex).

### SUMMARY OF THE DISCLOSURE

This disclosure relates to novel compositions for the treatment of shingles that utilize a cannabinoid-based nanoplatform designed for enhanced bioavailability and controlled release. These compositions integrate active ingredients such as cannabinoids, flavonoids, polyphenols, and alkaloids, which provide synergistic effects for treating the symptoms of shingles. The general formulation includes cannabinoids like cannabidiol (CBD) and cannabigerol (CBG), along with supportive agents like quercetin, resveratrol, capsaicin, and aloe vera. These ingredients work together to provide both systemic and localized relief, targeting inflammation, pain, and viral activity associated with shingles.

The oral formulation is presented in capsule form and comprises a combination of cannabinoids, flavonoids, and polyphenols, along with stabilizing solubilizing agents. The capsule is designed to modulate the immune response, reduce systemic inflammation, and provide antiviral effects, particularly targeting nerve pain, inflammation, and viral replication during shingles outbreaks. The nanoplatform in the capsules ensures controlled release, allowing the active ingredients to provide long-lasting relief from both acute symptoms and postherpetic neuralgia.

The topical formulation, on the other hand, incorporates cannabinoids such as CBD and CBG, combined with capsaicin and aloe vera, in a cream designed for direct application to the affected areas. This cream provides localized relief from pain, itching, swelling, and rashes. Capsaicin interrupts pain signaling pathways, while aloe vera soothes irritated skin and promotes healing. The topical application works in tandem with the systemic effects of the oral capsules, offering a comprehensive treatment strategy for managing the diverse symptoms of shingles.

The disclosure refers also to methods of treatment for shingles involving administering both the oral capsule and topical cream in a coordinated regimen to provide comprehensive relief from symptoms. The oral capsule is taken once daily to deliver systemic anti-inflammatory, antiviral, and neuroprotective effects, targeting nerve pain, viral replication, and overall inflammation. Simultaneously, the topical cream is applied directly to the skin four times daily, providing immediate localized relief from pain, itching, and swelling. This dual approach ensures that the treatment addresses both the internal and external symptoms of shingles, offering rapid symptom alleviation while also preventing complications like postherpetic neuralgia. The treatment is typically carried out over a period of four weeks, but may be adjusted depending on the severity of the symptoms and the patient's response.

The disclosure also describes a kit for the treatment of shingles, comprising both the oral capsule and the topical cream. The kit is designed to offer an integrated treatment plan, with the oral capsule providing systemic relief and the topical cream offering targeted relief to the lesions. The kit also includes instructions for use, specifying the administration of capsules once daily and the topical cream four times daily, over a period of four weeks, for optimal therapeutic outcomes.

Finally, the method of production involves nano-emulsification techniques to create a nanoplatform formulation with particles smaller than 200 nm. This process ensures that the cannabinoids, polyphenols, and flavonoids are efficiently delivered, maintaining stability and controlled release. The production method is essential for maximizing bioavailability and therapeutic efficacy, ensuring that the active ingredients reach the intended areas of the body and provide sustained relief for shingles symptoms.

### BRIEF DESCRIPTIONS OF THE FIGURES

FIG. 1. Mechanisms of Action of Cannabinoids, Quercetin, Resveratrol, and Capsaicin in Shingles Treatment. This figure illustrates how cannabinoids (CBD, CBG), quercetin, resveratrol, and capsaicin work in systemic and topical formulations to treat shingles. Systemic Treatment: Cannabinoids, quercetin, and resveratrol reduce inflammation by interacting with CB1 and CB2 receptors, inhibiting pro-inflammatory cytokines (TNF-α, IL-6, IFNs). Quercetin and resveratrol also provide antioxidant support. Topical Treatment: Cannabinoids, capsaicin, and aloe vera in topical formulations offer local relief. Capsaicin activates the TRPV1 receptor, reducing pain through desensitization and inhibiting viral replication. Aloe vera soothes and moisturizes, enhancing the anti-inflammatory effects of cannabinoids. This combination addresses inflammation, pain, and viral replication, offering a comprehensive approach to managing shingles symptoms.

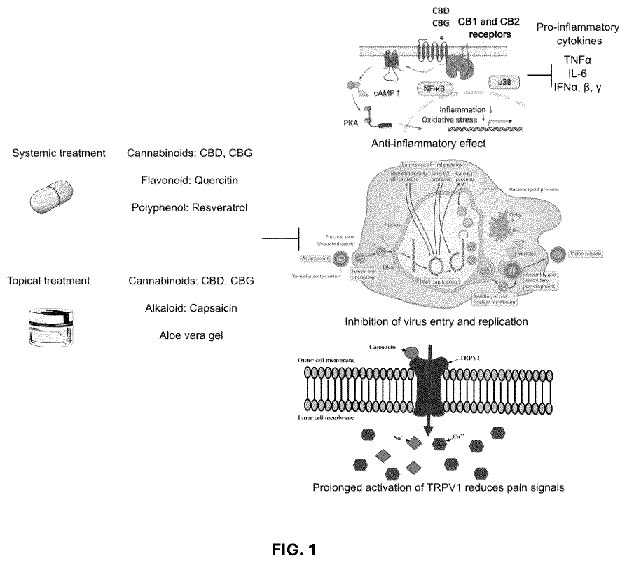

### DETAILED DESCRIPTION

### Definitions

The Varicella-zoster virus (VZV), also known as Human Herpesvirus 3 is a ubiquitous linear double-stranded DNA. Each particle has a diameter of approximately 150-200 nanometers and consists of an icosahedral nucleocapsid surrounded by a lipid envelope (Wang et al., 2020). This neurotropic alphaherpesvirus causes varicella (chickenpox) and Herpes Zoster (HZ) (Gershon A et al., 2015). Upon virus entry, the viral capsid containing the genome is transported to the nucleus by molecular motors along the microtubule system, where the genomic DNA is then released for replication within the nuclear viral factory (Bozzola & Bozzola, 2016).

The newly synthesized viral genome is packaged into the nascent capsid layer, which is subsequently exported from the inner nuclear membrane through egress. Following maturation, progeny varicella-zoster virus virions bud into the plasma membrane but remain associated on the cell surface, a phenomenon unique among all herpesviruses. At least three types of intermediate capsid particles (capsids A, B, and C), corresponding to different assembly states, have been isolated from herpes virus-infected cells (Quinlivan & Breuer, 2014). Capsids A consist of empty protein shells resulting from abortive DNA packaging. Capsids B comprise a proteinaceous core formed by scaffold proteins that support early capsid assembly (Cohen, 2010).

It is postulated that scaffold proteins are released after proteolytic maturation, enabling genomic DNA packaging to produce the complete capsid particle (capsid C). In addition to protecting genomic DNA, the viral capsid also facilitates its latent infection in neurons until it reaches the cell bodies in the sensory ganglia to maintain latency. Upon conditional reactivation, the capsid is subsequently transported from neuronal cell bodies to innervated dermatomes in an anterograde manner, leading to herpes zoster (shingles) (Perciani et al., 2017).

Understanding the structure of viral capsids and their assembly mechanisms provides a crucial foundation for the development of antiviral therapies (Perciani et al., 2019). In varicella-zoster virus (VZV), several open reading frames (ORFs) play critical roles in viral replication and immune evasion. The ORF47 kinase helps assemble the virus by guiding important proteins to the right places inside infected cells. It also weakens the immune response by reducing the activation of interferon (IFN) pathways. ORF66 contributes similarly by blocking the activation of immune-related genes. ORF63 is essential during latency, where the virus hides in sensory ganglia. Deleting ORF63 impairs the viruses ability to replicate and establish latency. ORF63 also regulates viral and host genes, particularly by controlling the major viral activator ORF62, which could be important for balancing virus activity during infection and latency.

Varicella-zoster virus (VZV), originally known for its neurotropic nature, has been found to also attack T cells. T cells or T lymphocytes are a type of white blood cell or defense cell that is part of the immune system and helps protect the body from infections and cancer. Classified as CD3+, CD4+, CD8+ and dual CD4+CD8+ T cells, they are highly susceptible to VZV replication and infection. This virus effectively infects tonsillar T cells, probably spreading from respiratory epithelial cells to T cells in lymphoid tissues such as the tonsils and Waldeyer ring. In addition, VZV can infect dendritic cells, facilitating its dissemination to lymph nodes (Huch et al., 2010). Infected CD4+ T cells often display a memory phenotype, expressing activation markers and skin-localizing proteins such as cutaneous leukocyte antigen (CLA) and chemokine receptor 4 (CCR4). This suggests their potential to migrate through the skin and other tissues (Zerboni et al., 2014).

After viral spread through the bloodstream, infected T cells eventually carry the varicella-zoster virus to epidermal cells of the skin and mucous membranes. Here, the virus replicates, causing typical vesicular lesions, and is shed in droplets from the respiratory tract. Latency is established in innervated neurons, triggering immediate immune responses. Glycoproteins gE and gI play crucial roles in T-cell infection. While gE large amino-terminal region (amino acids 1-188) is essential for replication, specific residues (51-187) facilitate virion envelopment and T-cell infection. Although gE usually forms a heterodimer with gI, this interaction is not required for T-cell infection. Furthermore, residues (27-90) on gE bind to insulin-degrading enzyme (IDE), enhancing VZV replication in melanoma cells, but are not essential for T-cell entry (Berarducci et al., 2010).

Varicella zoster virus (VZV)—specific T cell responses are believed to be vital in recovery from primary VZV infection and also in the prevention of viral reactivation. While glycoprotein E (gE) is the most abundant and one of the most immunogenic proteins of the virus, there is no data addressing potential T cell epitopes within gE, nor the phenotype of specific T cells. After airborne contamination and initial infection of the conjunctiva and respiratory mucosa of the upper airways, the varicella-zoster virus (VZV) undergoes its first replication cycle in lymph nodes (2nd-4th day) followed by a first viremia (4th-6th day). Subsequently, a second viral replication cycle in the reticuloendothelial system leads to systemic dissemination via a second viremia throughout the body.

Resident skin cells and recruited dendritic cells produce interferon-alpha and interferon-beta (IFN-α and IFN-β) as the initial responders to the infection. Natural killer (NK) cells are also activated early on, presumably to eliminate infected cells. The appearance of varicella-zoster virus-specific T-cells coincides with the resolution of lesions and is accompanied by sharp increases in specific IgM and IgG antibodies. (Freer and Pistello, 2018).

Post-acute infection, varicella-zoster virus persists in neurons, periodically reactivating. Subclinical reactivations of endogenous infection and occasional external contacts with chickenpox maintain immunity, thus offering long-term protection against reinfection and clinically evident reactivation. However, immunity declines with age, potentially leading to shingles if periodic reactivation falls below critical anti-varicella-zoster virus immunity thresholds (Weinberg et al., 2017). Young individual ability to mount rapid cellular responses to varicella-zoster virus reactivation correlates with protection against shingles, inversely correlating with disease severity and viremia.

It is thought that the expression of different co-stimulatory and lymph node homing molecules on virus-specific T cells correlates with differentiation and functional characteristics. Recently, it has been shown that IE63-specific CD4+ T cells express early intermediate differentiation markers, and up to 30% showed evidence of recent activation (Arvin et al., 2010). This could be explained by frequent re-exposure or reactivation, or alternatively, since IE63 is expressed during latency, it might reflect T cell exposure to a latently expressed protein. Distinguishing between these possibilities is important for understanding the pathogenesis of the disease and the critical components of immune control of latency. On the other hand, age-related decline in varicella-zoster virus-specific T cell immunity has been implicated in the occasional reactivation of the virus, leading to shingles.

Infection induces a strong humoral response (IgM, IgG, and IgA) and cellular immune responses. It is believed that antibodies persist for life, protecting against reinfections, as evidenced by the protective effect of varicella-zoster virus-specific medications as prophylaxis. Early production of IgG or IgM does not correlate with the severity of primary clinical infection or the onset of shingles, but post-vaccination increases are an excellent marker for protection against recurrent shingles (Gilbert et al., 2014). Shingles or herpes zoster, is an infection that causes a painful rash that usually appears on one side of the face or body and is characterized by swellings that crust over within 7 to 10 days and disappear within 2 to 4 weeks. Symptoms such as pain, itching, or tingling in the affected area usually occur before the rash. Other common symptoms include fever, headache, chills, and upset stomach.

The classification of Herpes Zoster represents 3 stages in which the infection develops and its clinical manifestation. The first pre-eruptive phase presents tingling, variable pain (mild to severe) and paresthesias in the affected area 1 to 4 days before the rash appears. Non-cutaneous symptoms such as headaches, malaise and sensitivity to light (photophobia) may also be present. The second acute eruptive phase is characterized by patchy erythema, occasionally accompanied by indurations, in the dermatomal area, regional lymphadenopathy; at this stage or later, herpetiform vesicles develop grouped on the erythematous base; cutaneous findings usually appear unilaterally, stopping abruptly at the midline of the affected dermatome; vesicles are initially transparent but eventually become cloudy, rupture, crust and involute.

The remaining erythematous plaques resolve slowly, usually without visible spasm. Scarring may occur if deeper dermal and epidermal layers have been affected by excoriation or secondary infection. Almost all adults experience pain. The pain may remain the same as in the prodrome or may change in character and intensity. Symptoms usually resolve in 10 to 15 days. Complete healing of the lesions may require up to a month or longer. The last phase corresponding to chronic phase (CPH) is characterized by persistent or recurrent pain lasting 30 or more days after acute infection and is usually confined to the area of the original dermatomal involvement. The pain can be severe and disabling; it may persist for weeks, months, or years, which is especially common in the elderly (>60 years). Patients experience complaints such as dysesthesias, paresthesias, and dizziness.

Complications of herpes zoster infection represent a medical problem that arises during the course of the disease, such as dermatological (keloids and secondary dermatoses, erythema multiforme, vasculitis) neurological (Guillain-Barre syndrome or stroke may occur, meningitis), ophthalmological (corneal ulcers and vision loss may occur) or visceral (pneumonia). Postherpetic neuralgia (PHN), is the most frequent complication, characterized by persistent and often debilitating pain that persists after the rash has healed.

The diagnosis of herpes zoster in patients is primarily based on clinical evaluation, focusing on the characteristics of the rash, its morphology, and typical distribution. Sometimes, diagnosis can be made even before the rash appears if patients exhibit typical pain in a dermatomal distribution. Essential diagnostic techniques for detecting herpes zoster include polymerase chain reaction (PCR), direct fluorescent antibody (DFA) testing, fluorescein eye staining, viral culture, and the Tzanck smear (Frisch and Guo, 2013).

The Tzanck smear is a diagnostic test used in dermatology to help identify certain viral skin infections, such as those caused by herpes simplex (HSV) or herpes zoster (varicella-zoster). Standard HZ treatment includes acyclovir (ACV), its prodrug valacyclovir, and brivudine, with valacyclovir offering higher bioavailability. ACV resistance can occur due to viral mutations, with famciclovir and valganciclovir as alternatives. New treatments like the helicase-primase inhibitor amenamevir, approved in Japan, and the ethanol extract of Elaeocarpus sylvestris (ES) show promise. For prevention, ACV or low-dose famciclovir is commonly used in immunosuppressed patients (Patil et al., 2022).

HZ vaccines, including the live attenuated VZV vaccine (Zostavax) and the recombinant adjuvanted VZV glycoprotein E subunit vaccine (Shingrix), aim to prevent HZ and PHN. Shingrix is more effective and safer for immunocompromised individuals. Clinical trials confirm the efficacy of both vaccines, with Shingrix inducing a strong immune response lasting over 12 months. Mass vaccination could significantly reduce HZ cases and related healthcare burdens. A US study estimated Shingrix effectiveness at 85.5%, with a large reduction in HZ incidence among vaccinated individuals (Patil et al., 2022).

Endocannabinoids regulate immune functions and maintain immune homeostasis. Immune cells express both CB1 and CB2 receptors, secrete endocannabinoids and possess mechanisms for cannabinoid transport and degradation. Studies indicate that human peripheral blood immune cells exhibit varying levels of cannabinoid receptor expression, with the rank order as follows: B cells>NK cells>monocytes>polymorphonuclear neutrophils>CD8 lymphocytes>CD4 lymphocytes.

CB1 receptors are predominantly found in the central nervous system, where they mediate neurobehavioral effects. In contrast, CB2 receptors are expressed in immune cells at levels 10-100 times higher than CB1 receptors. Furthermore, CB2 receptor mRNA has been detected in the cortex of lymph nodes and the nodular corona of Peyer patches, indicating a significant role in immune cell function and localization. Activation of the CB1 receptor is crucial for effective innate immune responses during viral and bacterial infections, while CB2 activation prevents additional damage from inflammatory processes such as sepsis—a potentially fatal body overreaction to infection causing tissue damage, organ failure, and death—through immunosuppressive effects.

AEA (anandamide or N-arachidonoylethanolamine) is synthesized from membrane phospholipid precursors mainly by the sequential action of N-acyltransferase (NAT) and Nacyl-phosphatidylethanolamines-specific phospholipase D (NAPE-PLD). 2-AG is synthesized by two diacylglycerol lipases (DAGL α/β). The endocannabinoids bio-activities are terminated by their fast catabolism, mainly through hydrolysis by a fatty acid amide hydrolase (FAAH), for AEA, and by a monoacylglycerol lipase (MAGL), for 2-AG. AEA has been demonstrated to have a high affinity for CB1 and a low CB2 affinity, while 2-AG has a moderate affinity for both receptors. (Dotsey et al, 2017).

Biochemical evidence highlights the significant role of endocannabinoids in immune and inflammatory modulation, with biosynthesis, uptake, and degradation occurring in macrophages and leukocytes. Studies show that RBL-2H3 basophil cells and J774 macrophages can biosynthesize and sequester AEA (anandamide) and PEA (palmitoylethanolamide), with these cells utilizing the same inactivating enzyme (FAAH) for hydrolysis. LPS (lipopolysaccharide) down-regulates FAAH expression, increasing AEA levels in human peripheral lymphocytes, suggesting FAAH inhibition as the mechanism.

Additionally, LPS exposure raises 2-AG (2-arachidonoylglycerol) levels in rat platelets and macrophages, indicating immune cells role in regulating peripheral endocannabinoid homeostasis. Human mast cells also hydrolyze AEA via FAAH, impacting inflammation and vascular tone. CB1 and CB2 receptors (cannabinoid receptors) regulate adenylyl cyclase activity and MAPK (mitogen-activated protein kinase) pathways, crucial for immune homeostasis.

CB2 receptor activation shows anti-inflammatory effects across various conditions, and the transient impact of cannabinoids on immune functions suggests minimal side effects, enabling immune activation during infections. Current data suggest that endocannabinoid signaling in lymphoid tissue provides tonic control over immune cell activation, maintaining immune homeostasis, with recent studies underscoring the dual role of endocannabinoids in immune regulation and homeostasis.

Furthermore, the interaction between the immune system and the nervous, immune, and endocrine systems forms the neuroimmunoendocrine network, with the endocannabinoid system (ECS) playing a pivotal role. Endocannabinoid system (ECS) activation is critical in modulating immune responses to infectious diseases, though its effects can be complex and multifaceted (Hernandez-Cervantes et al., 2017). In vitro evidence indicates that cannabinoids can influence infectious agents by either promoting replication or eliminating them, suggesting that cannabinoid receptors on pathogens might be responsible for these effects. This highlights a novel biological function of ECS activation. The immune system role in pathogen clearance, coupled with the expression of cannabinoid receptors on immune cells, underscores the potential impact of ECS modulation on health outcomes. Exploring these dynamics is vital to developing effective therapeutic strategies and enhancing our knowledge of immune responses.

Viral infections play a significant role in human diseases and despite advances in immunization and drug development, many viruses lack preventive vaccines and effective antiviral therapies, often hindered by the generation of viral escape mutants (Lin et al., 2014). Herbal medicines and purified natural products offer a valuable resource for the development of new antiviral medications. Throughout millennia, plants have been humanity primary resource for addressing bodily afflictions, preventing illnesses, and even diagnosing them. Pharmacognosy continues to serve as the primary means of identifying compounds with medicinal properties within plant materials: alkaloids, phenolics, polyphenols, flavonoids, quinones, tannins, coumarins, terpenes, lectins and polypeptides, saponins are among the primary compounds known for their antiviral properties.

The identification of antiviral mechanisms of these natural agents has shed light on how they interact with the viral life cycle-entry, replication, assembly, and viral release—as well as specific virus-host interactions (Lin et al., 2014). Therefore, the following is a brief overview of the antiviral activities of various natural products and medicinal herbs against herpesviruses (Xu et al., 2022). Additionally, more than 90% of adults have been infected with at least one human herpesvirus, which establishes long-term latent infection for the life of the host. While antiviral drugs exist that limit herpesvirus replication, many of these are ineffective against latent infection. Moreover, drug-resistant strains of herpesvirus emerge following chemotherapeutic treatment. For instance, Mutations in either HSV thymidine kinase or DNA polymerases can lead to the development of resistance to acyclovir and related nucleoside analogs (Son et al., 2013).

Therefore, there is an unmet medical need to develop new agents against herpesviruses with diverse mechanisms of action. Natural products provide a particular avenue to discover potent antiviral agents with significant pharmacological effects. Current science is turning to natural substrates, thus returning to the origins of pharmacy and botany, which were once united in the apothecary to address health problems of the time. It is for this reason that faced with viral and bacterial resistance that threatens us today, it is imperative to discover new signaling pathways, new therapeutic targets, new plant molecules, or all three if possible, to provide a comprehensive approach to the pathology, in this case, herpes zoster virus.

Currently, approximately 25% of prescribed drugs are derived from plants. Traditional herbal remedies have been used since ancient times, particularly in Asia, to treat various human and animal ailments. Even today, many parts of the world rely on traditional plant-based medicines for primary healthcare needs. Various bioactive phytochemicals such as polyphenols, alkaloids, flavonoids, saponins, quinones, terpenes, proanthocyanidins, lignins, tannins, polysaccharides, steroids, thiosulfonates, and coumarins have shown efficacy in the treatment of viral infections.

Polyphenols, such as resveratrol in grapes and berries, have demonstrated antiviral properties by inhibiting viral replication and modulating immune responses. Alkaloids like berberine from plants such as Berberis species exhibit antiviral effects against HZV by interfering with viral attachment and entry into host cells. Flavonoids, abundant in fruits and vegetables, including quercetin and catechins, have shown inhibitory effects on HZV replication through various mechanisms, including modulation of cellular signaling pathways critical for viral replication. These natural compounds, alongside other bioactive phytochemicals like proanthocyanidins, lignins, tannins, polysaccharides, steroids, thiosulfonates, and coumarins, collectively present a rich resource for developing novel therapeutic strategies against herpes zoster virus infections, leveraging both their antiviral potency and their potential to mitigate viral resistance mechanisms (Shinde et al., 2020).

Plants are reported to have been traditionally used as analgesics or resources for compounds with pain-relieving effects. For instance, opiate receptor agonists from poppy seeds and cyclooxygenase inhibitors from willow bark are widely used to alleviate pain in ancient medicine. Chili is also one of the sources of analgesic medications derived from plants. The ‘chili’ or ‘chili pepper’ plant, which is categorized under the genus Capsicum, belongs to a dicotyledonous group of flowering plants. The taxonomic position of Capsicum can be represented as follows: Kingdom: Plantae; Division: Magnoliophyta; Class: Magnoliopsida; Order: Solanales; Family: Solanaceae; Genus: Capsicum; Species: chinense/annuum/pubescens, among others.

Since ancient times, chili has been recognized for its broad range of therapeutic properties and has been used for centuries to remedy pain. Several external and internal applications have been reported in various streams of traditional medicine. Externally, it is used to treat different types of pain, including rheumatism (joint pain), lumbago (lower back pain), and neuralgia (pain spread through nerves). It can also be used as a local stimulant, counter-irritant, and anti rubefacient. In particular, neuropathic pain is a major form of chronic pain. This type of pain results from dysfunction or lesions in the central and peripheral nervous system. Capsaicin has been traditionally utilized as a medicine to remedy pain. However, the safety effectiveness of this practice is still elusive. (Yong, 2017).

To better comprehend the diverse effects of capsaicin, research has concentrated on its role in pain induction mechanisms. Capsaicin is recognized as a selective ligand for the TRPV1 receptor. When it binds to these receptors, it triggers the sensation of burning. The analgesic effect of capsaicin is thought to result from its capacity to induce reversible desensitization or defunctionalization, in which sensory axons containing TRPV1 become unresponsive to stimuli during a prolonged refractory period. (Yong, 2017).

Natural products have long been explored for their potential in combating herpes viruses, leveraging their diverse bioactive compounds. Extracts from medicinal plants, such as Melissa officinalis (lemon balm), and compounds like polyphenols from green tea have shown promising antiviral activity against HSV (Son et al., 2013). These natural agents often target various stages of viral replication, including viral entry, replication, and assembly, offering a multi-faceted approach to treatment. Research continues to uncover new natural sources and compounds with potential efficacy against both drug-sensitive and drug-resistant strains of HSV, highlighting their importance in the search for alternative therapies (Sand et al., 2021).

Plant extracts have garnered attention in the search for new molecules with antiherpetic activity. Interestingly, numerous extracts and compounds derived from plants have been reported to inhibit herpesvirus replication. For instance, organic extracts from Peganum harmala have been described to exhibit antiviral activity against herpes simplex virus type 2 (HSV-2) by interfering with virus entry (Benzekri et al., 2018). Essential oils extracted from plants of the Labiatae and Verbenaceae families have also demonstrated antiviral activity against herpes simplex virus (HSV). Vero cells incubated with HSV and essential oils from these plants for 48 to 72 hours significantly reduced viral titers of HSV-1 and HSV-2. Interestingly, their mechanisms of action were found to be related to pre-infectious stages (Brand et al., 2016).

Additionally, natural products have emerged as promising candidates for targeting various stages of the herpesviral life cycle. Firstly, they inhibit viral adsorption and entry by disrupting interactions between viral glycoproteins (e.g., gC and gD) and their cellular receptors (e.g., heparan sulfate, Nectin-1), crucial for viral attachment and fusion. Compounds like berberine, biliverdin, baicalin, and epigallocatechin-3-gallate have shown potent inhibitory effects against drug-resistant strains, suggesting potential therapeutic advantages without significant cytotoxicity (Ruchawapol et al., 2021).

Thirdly, certain natural compounds like berberine, wogonin (flavonoid obtained to Scutellaria baicalensis), and resveratrol inhibit NF-κB activation, pivotal for regulating inflammatory and apoptotic responses during infection. By interfering with NF-κB signaling, these compounds disrupt viral replication and enhance programmed cell death, potentially offering therapeutic benefits against herpesviruses (Ruchawapol et al., 2021). Aloe vera natural antioxidants and vitamins also support skin health, potentially aiding in minimizing scarring and promoting regeneration of damaged tissues. Therefore, it is important to identify which natural products are more efficacious anti-herpetic agents, and to understand the molecular mechanism in detail for further advance in the antiviral therapies.

Cannabis sativa is well documented to contain a rich array of secondary metabolites, including cannabinoids, terpenes, and phenolic compounds, which exhibit diverse pharmacological properties. With more than 400 active compounds that have therapeutic properties, cannabis has been accepted widely as a medical treatment and for recreational purposes in several countries. The compounds offer a range of clinical benefits, including but not limited to anticancer, antimicrobial, and antioxidant effects. The rise of antimicrobial resistance has become a significant global health challenge, highlighting the need for new drug discoveries. Cannabis sativa contains numerous chemical constituents produced through secondary metabolism, showcasing strong antimicrobial and antiviral properties.

A wide variety of compounds have been studied, with numerous research papers indicating that cannabinoids like cannabidiol and delta-9-tetrahydrocannabinol exhibit antiviral properties (Lin et al., 2014). The compounds present in cannabis show promising prospects as novel antibiotic candidates and natural antimicrobial agents for industrial and agricultural applications, addressing the urgent need for effective treatments against resistant microorganisms (Mahmud et al., 2021).

These compounds have shown efficacy in treating conditions such as anticonvulsant therapy, appetite stimulation, neurodegenerative diseases, pain management, skin disorders, and infectious diseases. Research highlights cannabinoids and terpenes, enriched in essential oils (EO), for their notable anti-inflammatory effects during infectious diseases. Approximately 545-550 compounds have been identified from C. sativa, including about 177 phytocannabinoids, 200 terpenes, and a similar number of phenolics (Mahmud et al., 2021).

Research underscores the pharmacological potential of cannabinoids in treating pain, colitis, spasticity, nausea, anorexia, sleep disorders, anxiety, epilepsy, and Alzheimer disease through preclinical and clinical studies (Mahmud et al., 2021). The immunomodulatory effects of cannabinoids via CB1 and CB2 receptors, densely located in immune tissues, nervous tissues, and the brain, are well-documented, prompting extensive discussion in scientific literature on their role in infectious diseases. Moreover, cannabinoids, extracts, and essential oils from C. sativa demonstrate antimicrobial activity against a range of pathogenic bacteria and fungi, supported by various studies, with emerging evidence suggesting efficacy against certain viruses as well (Mabou et al., 2020; Mahmud et al., 2021).

Throughout most of the last century, legal restrictions hindered the cultivation of hemp, isolation of cannabinoids, and research into their antiviral properties. Significant progress in the discovery of antiviral agents only emerged with the identification of antiviral targets through molecular biology in the 1980s. Since then, cannabinoids have demonstrated activity against DNA-type herpes viruses and RNA-type viruses such as HIV/SIV. CBD and Δ9-THC are the most studied antiviral cannabinoids, approved by the FDA for other pharmacological uses, and function through multiple mechanisms, including inhibition of the SARS-CoV-2 main protease 3CLpro and ACE2 receptor (van Breemen and Simchuk, 2023). These cannabinoids also exhibit anti-inflammatory properties beneficial for suppressing the pro-inflammatory effects of viruses. Other cannabinoids, such as CBDA and CBGA, have shown potential in preventing cell entry and infection by viruses like SARS-CoV-2. Research on the antiviral properties of over 100 less abundant cannabinoids is ongoing, with the potential for discovering even more potent agents (van Breemen and Simchuk, 2023).

Clinical trials are essential to explore the safety and efficacy of antiviral cannabinoids, with an emphasis on combination therapies due to their multiple mechanisms of action, which can slow the development of resistant virus strains. Phase 1 trials should determine appropriate dosing routes, levels, and frequencies to achieve therapeutic plasma concentrations. Additionally, these trials should consider possible interactions between cannabinoids and other drugs. Phase 2 clinical trials, designed to be prospective, placebo-controlled, randomized, double-blind, and sufficiently large, are necessary to establish the safety and efficacy of antiviral cannabinoids. These trials should use efficacious dosages to avoid failures seen in previous studies using subtherapeutic levels. Combinations of cannabinoids may exhibit synergistic effects at lower concentrations, as seen in other pharmacological activities (van Breemen and Simchuk, 2023).

While the antiviral activities of some cannabinoids have been documented in various models, studies on the less abundant cannabinoids and carefully designed clinical trials are still needed. Given the preclinical evidence and the long history of safe human use of cannabinoids, multiple clinical studies on the safety and efficacy of antiviral cannabinoids like CBD and Δ9-THC are underway globally, with more studies expected to follow (van Breemen and Simchuk, 2023). Research on the direct antiviral activity of Cannabis sativa compounds is limited. Instead, studies focus on the effects of cannabinoid receptors, CB1 and CB2, in human cells, which mediate the medicinal and psychoactive effects of cannabinoids in viral infectious diseases. The CB1 receptor, primarily found in the central nervous system (CNS), mediates psychoactive effects and significantly impacts viral infections in neural, lung, and liver tissues.

During viral infections, CB1 receptor activation can reduce cellular Ca2+ ion concentrations, impairing Ca2+-dependent enzyme release, nitric oxide (NO) production, nitric oxide synthase (NOS1), and pro-inflammatory mediators, which typically enhance host responses and inhibit viral replication. Conversely, CB2 receptors, mainly expressed in immune cells, play roles in immunomodulatory and inflammatory processes. CB2 activation can alter immune responses, suppress inflammation, regulate cytokine production, and mediate immune cell migration (Mahmud et al., 2021).

Cannabinoid receptor activation can regulate viral pathogenesis by modulating host inflammatory and immune responses. Although this activation can decrease viral pathogenesis in many infections, the literature shows considerable contradictions regarding the effects of cannabinoids. The signaling of cannabinoids can modify epigenetic changes by blocking the expression of genes involved in cell-virus interactions, altering virus entry into cells, replication, production, and host inflammation. However, studies conducted in vitro and in vivo indicate that the therapeutic use of immunoregulatory cannabinoids can sometimes result in disease progression, increased morbidity, and even host death by suppressing antiviral immune responses.

Therefore, in certain viral infections, blocking CB2 receptors is targeted to inhibit immune-suppressive effects. Further research is needed to fully understand the potential antiviral activities of cannabinoids and their implications in treating viral diseases (Mahmud et al., 2021). Numerous direct and indirect antiviral effects of cannabinoids have been observed both in vitro and in vivo. Activation of CB2 receptors by cannabinoid agonists suppresses replication of human immunodeficiency virus (HIV)-1. In vitro studies have highlighted CBD ability to inhibit hepatitis C virus (HCV) replication by 86.4% at a concentration of 10 μM. CBD inhibited the proliferation of KSHV-infected human microvascular endothelial cells (HMVEC) without affecting the virus ability to infect HMVECs when pretreated for 48 hours. CBD also induced greater apoptosis in infected cells compared to normal endothelium in a dose-dependent manner.

However, while cannabinoids anti-inflammatory activity shows promising therapeutic potential, especially in mitigating host immune responses to pathogens, direct evidence of C. sativa extracts inhibitory effects on viruses remains limited in the literature. Further preclinical trials are necessary to fully elucidate their antiviral capabilities (Mahmud et al., 2021). Cannabinoids, terpenes, and flavonoids are being investigated for their potential in shingles treatment due to their combined therapeutic effects. Cannabinoids like CBD (cannabidiol) have anti-inflammatory and analgesic properties that can help reduce pain and inflammation associated with shingles.

Terpenes, aromatic compounds found in cannabis and other plants, could add additional anti-inflammatory and analgesic effects, potentially enhancing overall therapeutic benefits. Meanwhile, flavonoids, also present in cannabis and various plants, have antioxidant and anti-inflammatory properties that could strengthen the treatment of shingles symptoms. Research into the antiviral activity of cannabinoids, terpenes, and flavonoids against shingles (herpes zoster) viral replication is an emerging area with preliminary findings suggesting potential therapeutic benefits. Research suggests that CBD might regulate immune responses and hinder viral replication in specific viruses. Nevertheless, there is limited specific research on its direct antiviral effects against the varicella-zoster virus (VZV), responsible for causing shingles, and more investigation is needed.

Flavonoids are phytochemicals with antioxidant, anti-inflammatory, and antiviral properties. They are abundant in many plants, including cannabis. Studies have indicated that certain flavonoids, such as quercetin and apigenin, exhibit antiviral effects by interfering with viral replication cycles or by enhancing immune responses against viruses. While their specific effects on VZV are not extensively studied, their broad-spectrum antiviral properties suggest potential relevance in shingles treatment.

The potential mechanisms of action of cannabinoids, terpenes, and flavonoids in the treatment of shingles (caused by the varicella-zoster virus, VZV) are not well established due to limited specific studies on these compounds against VZV. Nevertheless, considering their well-documented attributes and observed antiviral actions in various settings, we can postulate potential mechanisms by which they may influence the development of shingles:

Anti-inflammatory Effects: Cannabinoids, such as CBD (cannabidiol), are well-known for their anti-inflammatory effects. Inflammation is a key factor in the pain and discomfort linked to shingles. By influencing inflammatory responses, cannabinoids could help alleviate pain and swelling in the affected areas, thus enhancing the overall symptoms and quality of life for individuals with shingles.

Analgesic Effects: Cannabinoids have shown analgesic properties, meaning they can help alleviate pain. Shingles typically cause severe nerve pain (neuralgia) due to inflammation and nerve damage caused by VZV. Substances like CBD interact with cannabinoid receptors in the body endocannabinoid system and other pathways related to pain, potentially alleviating pain caused by shingles.

Modulation of Immune Responses: Flavonoids and cannabinoids have been studied for their immunomodulatory effects. In the case of shingles, where VZV reactivation from latency within sensory ganglia elicits immune responses, modulating these responses could affect the severity and duration of the infection. Flavonoids, such as quercetin, have been shown to inhibit inflammatory cytokines and enhance antiviral immune responses in other viral infections, which may have implications for managing shingles symptoms.

Antiviral Potential: While direct antiviral effects of cannabinoids, and flavonoids against VZV have not been conclusively demonstrated, some studies suggest that certain compounds (e.g., cannabinoids) may have broad-spectrum antiviral properties by affecting viral entry, replication, or assembly in other viruses. Further research is needed to determine if similar mechanisms could apply to VZV in the context of shingles.

Neuroprotective Effects: Cannabinoids have been investigated for their neuroprotective properties, which could be beneficial in mitigating nerve damage and pain associated with shingles. These effects might involve reducing oxidative stress, inflammation, and neuronal excitability in affected nerves.

Nanoemulsions are finely dispersed emulsions characterized by droplets with an average size ranging from 50 to 1000 nm. They are often referred to as ultrafine or submicron emulsions, appearing transparent due to their small size and exhibiting the Tyndall effect. Among their types, antimicrobial nanoemulsions are oil-in-water (o/w) systems stabilized by surfactants and co-surfactants like alcohols.

These formulations demonstrate effectiveness against a wide array of microorganisms, including viruses (e.g., herpes simplex, HIV), fungi (e.g., Candida, dermatophytes), and bacteria (e.g., Escherichia coli, Salmonella spp., Staphylococcus aureus) (Begum et al, 2024).

The mechanism of action involves the electrostatic attraction and thermodynamic fusion of nanoemulsion droplets with lipid-coated microorganisms. This fusion disrupts the pathogen lipid membrane, leading to cell lysis and microbial death.

Nanoemulsions offer several advantages, such as clarity, biocompatibility, non-immunogenicity, biodegradability, efficient drug encapsulation and controlled release, small particle size with a high surface area, ease of production, and thermodynamic stability. These properties make nanoemulsions promising candidates for various applications in biomedical and pharmaceutical fields (Begum et al, 2024).

Nanotechnology has emerged as a promising frontier for combating multidrug-resistant microorganisms. The emphasis has been on crafting nanoparticles with tailored physicochemical properties, aimed at improving drug delivery systems using precise carrier technologies.

Among these advancements, nanoemulsion systems stand out as highly effective in modern medicine, facilitating targeted and systemic drug delivery with enhanced therapeutic efficacy (Qian & McClements, 2011).

Nanoemulsions are structured with both lipophilic and hydrophilic components, forming well-defined 3D polymeric and lipid chains encapsulating therapeutic or diagnostic molecules. These systems boast superior drug encapsulation capabilities, ensuring precise delivery to specific receptor sites for optimal pharmacological action.

By optimizing the blend of surfactants and co-surfactants, nanoemulsions achieve favorable changes in their physicochemical characteristics, enhancing bioavailability and overcoming biological membrane barriers at disease sites. This innovative approach enhances drug transport efficiency and improves therapeutic outcomes across various biological environments (Begum et al, 2024).

Nanoemulsion systems represent the pinnacle of advanced drug delivery technologies, offering significant advantages over microemulsions and microemulsions. Unlike microemulsions, nanoemulsions excel in stability, preventing issues such as creaming, flocculation, and caking. They provide numerous benefits, including rapid absorption, consistent drug absorption profiles, enhanced solubilization of hydrophobic drugs, improved bioavailability, and versatility in administration routes, enhanced tissue penetration, and the ability to mask unpleasant drug tastes (Qian & McClements, 2011).

Moreover, nanoemulsions shield drug molecules within the lipid phase, guarding them against hydrolysis and oxidation, thus prolonging their shelf life and preserving their efficacy. Nanoemulsions formulated with chemotherapeutic and bioactive agents are pivotal in enhancing patient compliance and reducing the manufacturing energy footprint (Begum et al, 2024).

In the realm of combating viral infections, delivering effective medications has always posed challenges. Recent advancements in nanoemulsion technology have shown promising results in overcoming these hurdles. Traditionally, methods like oral or parenteral administration of antiviral drugs have drawbacks such as systemic toxicity, drug degradation, and limited therapeutic effects. Nanoemulsions, however, offer a promising alternative due to their ability to encapsulate drugs efficiently and deliver them precisely to target sites (Klang et al., 2010).

Studies have highlighted the effectiveness of nanoemulsions, particularly those containing silver and gold particles, against a variety of viral strains including HIV-1. Silver nanoemulsions have demonstrated potent antiviral activity by inhibiting viral replication steps and reducing the infectiousness of CD4+ cells. They exhibit minimal toxicity and high efficacy against HIV-1 strains, both in cell culture and in free viral forms (Begum et al, 2024).

Similarly, gold nanoemulsions equipped with ligands that bind effectively to HIV envelope proteins, have shown promise in inhibiting viral attachment and fusion with host cells. These properties make them effective in combating viral infections, including flu strains, through mechanisms that interfere with viral replication and cell entry processes (Begum et al, 2024).

Furthermore, polymeric gold nanoemulsions have proven effective against herpes simplex virus Type-1 (HSV-1), leveraging their ability to inhibit viral entry into cells. This targeted approach minimizes cytotoxicity while maximizing antiviral efficacy, demonstrating significant potential in treating infections like hepatitis B and other viral diseases (Paradowska et al., 2021).

In summary, nanoemulsion technology represents a transformative approach in antiviral drug delivery. Its ability to enhance drug stability, target-specific delivery, and minimize adverse effects holds great promise in revolutionizing the treatment of viral infections, addressing critical gaps in current therapeutic strategies (Begum et al, 2024).

### Pharmaceutical Compositions:

Aspects of the disclosure provide a method to treat the symptoms associated with shingles and support immune health using a nanoplatform composition comprising cannabinoids, flavonoids, polyphenols, and alkaloids. This composition takes advantage of the synergistic effects of these bioactive compounds to address both the underlying viral activity and the symptomatic manifestations of shingles, such as pain, itching, and inflammation. Cannabinoids, particularly cannabidiol (CBD) and cannabigerol (CBG), interact with the body's endocannabinoid system, modulating pain perception, reducing inflammation, and potentially inhibiting viral replication. By targeting the CB1 and CB2 receptors, cannabinoids not only alleviate nerve pain but also promote neuroprotective effects, which can be critical in reducing the long-term consequences of shingles, such as postherpetic neuralgia.

Flavonoids, such as quercetin, are incorporated for their well-documented anti-inflammatory and antioxidant properties. They help to mitigate oxidative stress and support the body's natural immune response, which is crucial during a viral infection like shingles. These compounds are known to enhance immune function, allowing the body to fight off viral reactivation more effectively, and they play a role in reducing the duration and severity of symptoms.

Polyphenols, such as resveratrol, add another layer of therapeutic benefit. Resveratrol has been shown to have antiviral properties by inhibiting the replication of viruses and reducing inflammation, further aiding in the management of shingles. Its antioxidant properties also help protect cells from oxidative damage, promoting faster recovery and healing of the skin lesions that typically accompany shingles.

Alkaloids, including capsaicin, are used primarily for their analgesic properties. Capsaicin works by desensitizing pain receptors, thereby reducing the burning and tingling sensations that are characteristic of shingles. This multi-compound approach, encapsulated in a nanoplatform, ensures enhanced bioavailability and targeted delivery, allowing the active ingredients to be absorbed more efficiently and exert their effects at both systemic and local levels. This method offers a comprehensive solution for treating shingles by addressing the pain, inflammation, and viral replication while supporting the immune system's ability to prevent further complications.

The disclosure, as described herein as embodied in the examples provided, is not limited to the details shown because various modifications and changes can be made without departing from the scope of the disclosure and the equivalent of the claims. The following description of specific embodiments will help clarify the construction, operation, and additional benefits of the disclosure. Aspects of the disclosure refer to a composition for treatment of shingles, comprising: a) an active principle comprising at least one cannabinoid; and b) a lipid agent.

In some embodiments, the active principle comprises at least one cannabinoid selected from a group that includes Δ9-Tetrahydrocannabinol (THC), Cannabidiol (CBD), Cannabigerol (CBG), Cannabinol (CBN), Tetrahydrocannabivarin (THCV), Cannabichromene (CBC), Delta-8-Tetrahydrocannabinol (Δ8-THC), Cannabidivarin (CBDV), Tetrahydrocannabinolic Acid (THCA), Cannabidiolic Acid (CBDA), Cannabigerolic Acid (CBGA), Cannabichromenic Acid (CBCA), or combinations thereof. Each of these cannabinoids brings distinct therapeutic properties, making their inclusion versatile and adaptable to the treatment of various symptoms associated with shingles and other conditions.

Δ9-Tetrahydrocannabinol (THC) is known for its psychoactive properties, but it also has potent analgesic and anti-inflammatory effects, which can be crucial in managing the pain and discomfort associated with shingles. It binds primarily to CB1 receptors in the central nervous system, making it effective in modulating nerve pain and reducing inflammation, which are common in shingles outbreaks.

Cannabidiol (CBD) is a non-psychoactive cannabinoid that has garnered significant attention due to its anti-inflammatory, analgesic, and neuroprotective properties. CBD interacts with both CB1 and CB2 receptors, helping to alleviate pain and reduce inflammation without causing the intoxicating effects of THC. It also has immunomodulatory effects, which can aid in controlling the immune system's response to the reactivation of the varicella-zoster virus. Cannabigerol (CBG), often referred to as the “parent cannabinoid” because it is the precursor to other cannabinoids like THC and CBD, is particularly known for its anti-inflammatory and neuroprotective properties. It may be especially beneficial for managing nerve pain and inflammation, which are key symptoms in shingles. Additionally, CBG has demonstrated potential in reducing intraocular pressure, providing further therapeutic options for conditions with ocular complications. Cannabinol (CBN) is another non-psychoactive cannabinoid that has mild sedative effects. It can be particularly useful in helping shingles patients manage sleep disturbances and discomfort caused by persistent pain. CBN has also shown anti-inflammatory and antibacterial properties, making it an effective complement to other cannabinoids in the formulation.

Tetrahydrocannabivarin (THCV) is a psychoactive cannabinoid, though its effects differ from THC, often described as less intoxicating and potentially more energizing. THCV has been studied for its ability to reduce inflammation and modulate blood sugar levels, providing benefits for immune response and metabolic health during infection recovery. Cannabichromene (CBC) is a non-psychoactive cannabinoid that has demonstrated anti-inflammatory, analgesic, and antiviral properties. It works synergistically with other cannabinoids to enhance the overall therapeutic effect, particularly in reducing pain and inflammation in skin conditions like shingles. Delta-8-Tetrahydrocannabinol (Δ8-THC) is a less psychoactive variant of THC that still offers potent analgesic and anti-inflammatory benefits, making it a potential option for patients who seek pain relief without strong psychoactive effects. It also binds to both CB1 and CB2 receptors, contributing to its therapeutic versatility.

Cannabidivarin (CBDV) is similar in structure to CBD and has shown promise in managing neurological conditions, reducing seizures, and potentially alleviating neuropathic pain. Its non-psychoactive nature, combined with its ability to interact with the endocannabinoid system, makes it a valuable component in managing the nerve pain commonly associated with shingles. Tetrahydrocannabinolic Acid (THCA) is the non-psychoactive precursor to THC. While it does not have the intoxicating effects of THC, it has shown anti-inflammatory and neuroprotective properties, contributing to pain relief and the reduction of inflammation in conditions like shingles. Cannabidiolic Acid (CBDA) is the acidic precursor to CBD. Although less studied, CBDA has demonstrated potential anti-inflammatory and anti-nausea properties, which may further support symptom relief in patients with shingles. Cannabigerolic Acid (CBGA) is the acidic precursor to CBG and is responsible for synthesizing other cannabinoids. CBGA exhibits anti-inflammatory and neuroprotective properties, supporting the reduction of inflammation and nerve pain. Cannabichromenic Acid (CBCA) is the acidic precursor to CBC, and like its decarboxylated form, it has demonstrated potential anti-inflammatory and antiviral properties. Including CBCA in formulations offers a broader spectrum of therapeutic benefits, targeting multiple pathways involved in pain and inflammation.

These cannabinoids can be combined in specific ratios to maximize therapeutic effects, leveraging the “entourage effect,” where cannabinoids work synergistically to produce greater efficacy in treating pain, inflammation, and viral symptoms associated with shingles. The composition can be tailored to address patient-specific needs, balancing the benefits of psychoactive and non-psychoactive cannabinoids to optimize pain relief, immune support, and recovery.

In various embodiments, the composition may vary in the concentration of cannabinoids to tailor the therapeutic response to the specific needs of the patient. This flexibility allows for precise dosing depending on the severity of symptoms, patient tolerance, and the desired therapeutic outcomes. For example, higher concentrations of cannabidiol (CBD) may be utilized for its potent anti-inflammatory and analgesic effects, making it effective in managing acute pain and inflammation associated with shingles. Conversely, lower concentrations of cannabinoids like tetrahydrocannabinol (THC) might be employed to minimize psychoactive effects while still providing therapeutic benefits, particularly in managing neuropathic pain or sleep disturbances.

In various embodiments, the composition may vary in the concentration of cannabinoids to customize therapeutic potency and enhance patient response, allowing for precise adjustments based on the specific symptoms of shingles, patient tolerance, and desired outcomes. This flexibility ensures that each formulation can be optimized to treat individual cases of shingles effectively, accounting for the variability in symptoms such as pain intensity, inflammation, and nerve damage. Patients with milder symptoms may benefit from lower cannabinoid concentrations, whereas those suffering from more severe outbreaks, characterized by intense pain or chronic postherpetic neuralgia, may require higher doses to achieve symptom relief.

For instance, higher concentrations of cannabidiol (CBD) may be employed to leverage its potent anti-inflammatory and analgesic properties, which are especially effective in reducing pain and inflammation associated with shingles outbreaks. CBD interacts with the endocannabinoid system, modulating the body's inflammatory response and reducing oxidative stress, which contributes to pain relief and skin healing. This makes it particularly valuable for patients experiencing acute phases of shingles, where rapid relief from inflammation and itching is crucial for improving comfort and preventing secondary infections from constant skin irritation.

Conversely, formulations with increased levels of cannabigerol (CBG) may be utilized to enhance neuroprotective effects, making them particularly effective for addressing nerve pain and discomfort that often accompany shingles. CBG has shown promising results in supporting nerve health and reducing neuroinflammation, which can be critical for patients experiencing sharp, stabbing pains or prolonged postherpetic neuralgia. By focusing on CBG's ability to protect nerve cells and minimize long-term nerve damage, these formulations aim to not only alleviate current symptoms but also prevent the development of chronic complications, thereby improving long-term patient outcomes.

In further embodiments, the composition may combine two or more cannabinoids, with a preference for cannabidiol (CBD) and cannabigerol (CBG), in carefully determined ratios to create a synergistic effect that amplifies their therapeutic potential. This synergy, commonly known as the “entourage effect,” refers to the phenomenon where cannabinoids work together to produce more significant health benefits than when used individually. By combining CBD and CBG in specific ratios, the composition can more effectively modulate various physiological processes, such as inflammation, pain, and immune response, leading to more comprehensive management of shingles symptoms.

For example, CBD is widely recognized for its potent anti-inflammatory and analgesic properties, which help to relieve pain and reduce the inflammation associated with shingles. Meanwhile, CBG offers neuroprotective benefits, targeting nerve damage and reducing neuroinflammation, which can be crucial for patients dealing with nerve pain, a common complication of shingles. When these cannabinoids are combined in a specific ratio, their complementary actions can enhance overall pain relief, making the composition more effective in both acute phases of shingles, where inflammation and pain are prominent, and in chronic phases, where postherpetic neuralgia becomes a concern.

By incorporating these cannabinoids together, the composition not only addresses pain and inflammation but also supports the healing of herpetic lesions and helps manage associated skin irritation and hypersensitivity. CBD's ability to soothe irritated skin, combined with CBG's neuroprotective and anti-inflammatory properties, creates a more robust therapeutic response. This dual-action approach not only alleviates immediate discomfort but also promotes faster healing and reduces the likelihood of long-term complications, such as scarring or chronic pain, thereby refining the composition's overall efficacy in treating shingles.

In further embodiments, the ratio of cannabinoids can be precisely adjusted within defined concentration ranges to tailor the composition to meet specific patient needs or therapeutic objectives. This flexibility allows for personalized treatment plans, where the balance of cannabinoids such as CBD and CBG can be fine-tuned based on the severity of shingles symptoms, patient tolerance, and the desired therapeutic outcome. For instance, patients experiencing more intense nerve pain may benefit from a higher ratio of CBG due to its neuroprotective properties, while those suffering from acute inflammation may require a higher concentration of CBD to reduce swelling and pain effectively. This adaptability ensures that the composition can provide targeted relief for both the immediate and long-term effects of shingles.

Moreover, the ability to adjust cannabinoid ratios makes it possible to create different formulations for various delivery systems, such as oral capsules for systemic relief and topical creams for localized symptom management. Capsules with a tailored cannabinoid composition can address systemic symptoms like widespread pain, immune modulation, and inflammation, offering sustained relief throughout the body. These systemic treatments are particularly effective for patients who experience severe or chronic shingles-related symptoms, including postherpetic neuralgia. The ratio of cannabinoids in capsules can be optimized to enhance bioavailability and therapeutic impact, ensuring that the active compounds are delivered efficiently to address the underlying causes of pain and inflammation.

On the other hand, topical creams with adjusted cannabinoid content provide direct relief to areas affected by shingles, targeting skin rashes, irritation, and localized pain. By tailoring the cannabinoid concentrations in topical formulations, the composition can better manage surface-level symptoms like itching, tingling, and skin sensitivity. This dual-delivery system, incorporating both systemic and localized treatments, offers a comprehensive and versatile approach to shingles care, ensuring that patients receive balanced and effective symptom relief tailored to their unique needs.

Additionally, The ratio of different cannabinoids within the composition can be fine-tuned to optimize its therapeutic profile, ensuring that efficacy is maximized while minimizing potential side effects. This flexibility allows for multiple cannabinoid combinations to be developed based on the patient's specific needs and the phase of shingles being treated. For instance, a high-CBD, low-CBG ratio might be ideal for cases where pain relief and inflammation reduction are primary concerns, as CBD's strong anti-inflammatory and analgesic properties would dominate the formulation. In contrast, a more balanced ratio of CBD and CBG could be employed to harness both the neuroprotective benefits of CBG and the pain-relieving effects of CBD, providing a more comprehensive approach to managing symptoms like nerve pain and viral activity.

Furthermore, the ratio of different cannabinoids within the composition can be adjusted to optimize the therapeutic profile, balancing efficacy with minimized side effects. This approach allows the composition to be customized, such as formulating a high-CBD, low-CBG ratio to enhance pain relief while reducing inflammation. By using a balanced ratio of CBD and CBG, the composition can further enhance its antiviral and neuroprotective effects, providing a targeted solution for shingles symptoms. The composition includes cannabinoids at a concentration ranging from 3% to 12%. In a preferred embodiment, the composition includes cannabinoids at a concentration ranging from 8% to 12%. In a further preferred embodiment, the composition includes cannabinoids at a concentration ranging from 3% to 4%, specifically tailored to address shingles-related discomfort and inflammation with minimal side effects.

In a preferred embodiment, a composition with a total cannabinoid concentration of 8% could include 7% CBD and 1% CBG, focusing primarily on inflammation reduction and pain relief while including CBG for mild neuroprotective benefits. This ratio would be ideal for patients seeking effective relief with minimal risk of side effects from higher concentrations of CBG. Similarly, a 10% total cannabinoid concentration might include 9% CBD and 1% CBG, allowing for a stronger anti-inflammatory effect while still incorporating CBG to address nerve pain and discomfort. A maximum concentration of 12% could consist of 11% CBD and 1% CBG, enhancing the formulation's overall effectiveness in managing severe pain and inflammation while maintaining a focus on minimizing neuroinflammation with CBG.

Alternatively, a more balanced cannabinoid ratio could be beneficial in addressing both systemic inflammation and chronic nerve pain, especially in patients suffering from postherpetic neuralgia. In this case, an 8% concentration might include 5% CBD and 3% CBG, providing stronger neuroprotective effects while still focusing on inflammation reduction. A 10% composition could be formulated with 6% CBD and 4% CBG, enhancing both cannabinoid effects for a more comprehensive treatment approach. In cases where a 12% total cannabinoid concentration is used, a balanced ratio of 7% CBD and 5% CBG could offer robust pain relief, anti-inflammatory effects, and neuroprotection, making it an ideal option for more complex or severe shingles symptoms.

In some instances, a composition specifically tailored for minimal side effects, such as 3% to 4% cannabinoid concentrations, could prioritize a lighter formulation while still providing effective relief. For example, a 4% cannabinoid concentration could include 3% CBD and 1% CBG, designed to focus on reducing shingles-related discomfort and inflammation without overwhelming the patient with high doses. This kind of customization, by varying both the total concentration and the ratios of cannabinoids, allows the composition to offer targeted, patient-specific treatment while maintaining flexibility in addressing a broad range of shingles symptoms.

In further embodiments, the composition may combine two or more cannabinoids in specific ratios designed to optimize therapeutic outcomes. For instance, a high CBD-to-CBG ratio may be utilized to emphasize CBD's anti-inflammatory properties while maintaining low levels of CBG to enhance neuroprotective effects with minimal psychoactivity. This combination would be particularly beneficial for patients seeking relief from inflammation and pain without the psychoactive effects typically associated with higher concentrations of cannabinoids like THC. The high-CBD formulation focuses on reducing inflammation and alleviating pain, which are primary symptoms of shingles, while the lower CBG content provides additional support in reducing nerve damage and discomfort, particularly in cases of postherpetic neuralgia.

For a more balanced approach, formulations could combine CBD and CBG in closer ratios to enhance both cannabinoids' benefits, such as pain relief, inflammation reduction, and neuroprotection. This balanced formulation would be ideal for patients suffering from both acute shingles outbreaks, characterized by skin rashes and inflammation, and the chronic nerve pain that can follow, such as in postherpetic neuralgia. By carefully selecting the ratios, the composition ensures that both systemic and localized symptoms are addressed effectively, providing a comprehensive solution for managing the complex symptom profile of shingles.

In addition to the primary cannabinoids, minor cannabinoids such as cannabinol (CBN) or cannabichromene (CBC) may be incorporated in precise quantities to further enhance the formulation's therapeutic potential. CBN, known for its mild sedative and pain-relieving properties, can be useful in treating shingles-related discomfort, particularly in improving sleep quality for patients dealing with chronic pain. CBC, on the other hand, has demonstrated anti-inflammatory and antiviral effects, making it an ideal complement to CBD and CBG in managing both the physical manifestations of shingles, such as rashes and inflammation, and the underlying viral activity. Including these minor cannabinoids adds another layer of therapeutic benefit, ensuring that the formulation addresses a wider range of symptoms and improves overall patient outcomes.

This multifaceted approach, combining primary cannabinoids like CBD and CBG with minor cannabinoids such as CBN and CBC, allows for a synergistic effect, where the overall therapeutic action is greater than that of the individual cannabinoids used in isolation. By leveraging the unique properties of each cannabinoid, the composition provides a more comprehensive therapeutic benefit, targeting various physiological pathways involved in shingles, from pain and inflammation to nerve damage and viral replication. This strategy enhances the management of shingles symptoms, ensuring that patients receive a holistic treatment that addresses both immediate discomfort and long-term complications.

Compositions of the disclosure may be presented in various pharmaceutical dosage forms, each tailored to provide optimal therapeutic outcomes for the treatment of shingles by addressing both systemic and localized symptoms. These versatile formulations ensure that different aspects of the condition, from widespread inflammation to localized pain, are managed effectively, offering patients a comprehensive treatment strategy. By utilizing multiple delivery systems, the composition can target various physiological mechanisms involved in shingles, enhancing the overall efficacy of the treatment.

In one embodiment, the pharmaceutical dosage form may include oral capsules, designed to provide systemic relief from the broader symptoms of shingles. These capsules can contain a carefully balanced combination of cannabinoids, such as cannabidiol (CBD) and cannabigerol (CBG), which work synergistically to reduce inflammation, alleviate pain, and modulate the immune response. The systemic action of these capsules makes them ideal for patients experiencing more generalized symptoms, such as nerve pain, fever, or fatigue, associated with the body's response to the varicella-zoster virus. By circulating throughout the body, the cannabinoids in the capsules can target widespread nerve inflammation and provide long-lasting relief, helping to prevent the development of postherpetic neuralgia, a common complication of shingles.

In another embodiment, the pharmaceutical dosage form may consist of a topical cream specifically designed for localized application to shingles lesions. This cream can be applied directly to affected areas, targeting symptoms such as pain, itching, and inflammation that occur around the skin rashes caused by shingles. The inclusion of cannabinoids like CBD and CBG, along with additional soothing ingredients such as aloe vera or capsaicin, provides targeted pain relief while promoting the healing of the herpetic blisters and reducing skin sensitivity. By delivering active ingredients directly to the site of inflammation, the cream offers quick, localized relief, helping to alleviate the discomfort caused by shingles without systemic exposure, which may be beneficial for patients with localized symptoms or those who are sensitive to systemic treatments.

These pharmaceutical dosage forms, both systemic and localized, allow for a comprehensive approach to managing shingles symptoms. The capsules address the internal, systemic effects of the virus, such as nerve inflammation and immune response, while the cream focuses on the external, localized symptoms, providing direct relief to the skin and affected nerve areas. By utilizing both approaches, the treatment offers flexibility in addressing a wide range of patient needs, from acute relief during an outbreak to ongoing management of chronic symptoms, such as postherpetic neuralgia, that can persist long after the initial shingles outbreak has subsided. This multi-pronged treatment strategy ensures that the composition can be tailored to the specific symptomatology and therapeutic goals of each patient.

In this embodiment, a pharmaceutical formulation is provided that integrates a second active principle comprising at least one polyphenol, one flavonoid, and one alkaloid, carefully chosen for their synergistic therapeutic effects on inflammation, immune modulation, and antiviral properties. The inclusion of these compounds enhances the overall efficacy of the formulation, providing a more comprehensive approach to managing shingles symptoms and promoting faster recovery.

Polyphenols, such as resveratrol, curcumin, epigallocatechin gallate (EGCG), and ellagic acid, are well known for their potent antioxidant and anti-inflammatory properties. Resveratrol, for example, has demonstrated antiviral effects by inhibiting viral replication and reducing oxidative stress, which is a critical factor in the inflammation associated with shingles. Similarly, curcumin, derived from turmeric, possesses both anti-inflammatory and immune-modulating effects, making it useful in reducing the inflammatory response and supporting the body's defense against the reactivation of the varicella-zoster virus. EGCG, a polyphenol found in green tea, also contributes to immune health and has shown antiviral activity, particularly against herpes viruses, which makes it highly relevant in the context of shingles treatment. Ellagic acid, another powerful polyphenol, adds further antioxidant protection, reducing cellular damage caused by oxidative stress during viral outbreaks.

Flavonoids, such as quercetin, luteolin, kaempferol, apigenin, and rutin, are included in the formulation for their anti-inflammatory, antiviral, and immune-supporting properties. Quercetin, for example, has been shown to inhibit the replication of several viruses, including those in the herpes family, while also reducing the release of pro-inflammatory cytokines. Luteolin and kaempferol further contribute to the anti-inflammatory effects, modulating the immune response to prevent excessive inflammation that can exacerbate shingles symptoms. Apigenin, known for its calming effects on the immune system, and rutin, which supports vascular health, help ensure that inflammation is kept under control while promoting healing in the affected tissues. These flavonoids work together to reduce the severity of shingles outbreaks, helping to alleviate pain and skin irritation.

The alkaloids included in the formulation, such as berberine, piperine, capsaicin, theobromine, and caffeine, offer additional therapeutic benefits, particularly in pain relief and immune modulation. Berberine has been studied for its broad-spectrum antimicrobial and antiviral effects, making it a valuable component for reducing the viral load during shingles outbreaks. Piperine, often used to enhance the bioavailability of other compounds, ensures that the active ingredients in the formulation are absorbed efficiently. Capsaicin, widely known for its ability to desensitize pain receptors, provides targeted relief from the nerve pain associated with shingles. Theobromine and caffeine, both found in cacao, contribute mild stimulatory effects, which can support overall well-being during recovery by improving circulation and reducing fatigue. Together, these alkaloids enhance the formulation's ability to manage pain, reduce inflammation, and support the immune system's response to shingles.

By combining polyphenols, flavonoids, and alkaloids, this pharmaceutical formulation provides a well-rounded approach to managing shingles. The active principles work in synergy to control inflammation, boost immune function, and reduce viral activity, resulting in improved symptom management and faster recovery for patients. This approach leverages the complementary actions of these natural compounds, ensuring that each element enhances the therapeutic effects of the others, leading to a more effective and holistic treatment.

In a further embodiment, the composition includes a second active principle composed of one flavonoid, one polyphenol, and one alkaloid, each selected for their distinct yet complementary therapeutic properties. This combination allows the composition to target multiple biological pathways, providing a multifaceted approach to treating shingles. The integration of these three types of bioactive compounds ensures that the formulation addresses key aspects of the condition, including inflammation, pain relief, immune modulation, and antiviral activity.

The chosen flavonoid, quercetin, is known for its powerful anti-inflammatory and antiviral properties. Quercetin works by inhibiting the production and release of pro-inflammatory cytokines, which are often elevated during shingles outbreaks. By reducing inflammation at a cellular level, quercetin helps to alleviate the swelling, pain, and irritation associated with shingles lesions. Additionally, quercetin's antiviral capabilities are particularly beneficial in managing shingles, as it can help inhibit the replication of the varicella-zoster virus, potentially reducing the severity and duration of the outbreak.

Resveratrol, the polyphenol in this preferred embodiment, is recognized for its antioxidant, anti-inflammatory, and antiviral effects. Resveratrol not only scavenges free radicals, reducing oxidative stress, but also modulates immune responses, making it a valuable component in managing both the acute and chronic phases of shingles. Its antiviral properties further support the reduction of viral replication, contributing to quicker recovery. Resveratrol has been studied for its ability to interfere with viral DNA synthesis, thus limiting the proliferation of the virus responsible for shingles. In addition to its direct effects on the virus, resveratrol helps improve overall skin health by promoting healing and reducing the risk of long-term scarring.

Capsaicin, the alkaloid in this embodiment, is widely known for its role in pain management, particularly in conditions involving nerve pain, such as shingles. Capsaicin works by desensitizing nociceptors, which are the pain receptors in the skin and nerves. This desensitization reduces the sensation of pain, especially the burning or tingling nerve pain often associated with shingles and postherpetic neuralgia. By including capsaicin in the composition, the formulation provides targeted relief from one of the most debilitating symptoms of shingles: persistent nerve pain. Capsaicin also exhibits mild anti-inflammatory properties, which complement the actions of quercetin and resveratrol, ensuring a comprehensive approach to managing both pain and inflammation.

Together, the combination of quercetin, resveratrol, and capsaicin in this embodiment forms a potent second active principle that works synergistically to reduce inflammation, modulate immune responses, relieve pain, and potentially inhibit viral activity. This balanced approach enhances the overall efficacy of the composition, making it a more robust treatment option for shingles by addressing both the immediate symptoms and the underlying viral mechanisms, all while supporting the body's natural healing processes.

In one embodiment, the pharmaceutical composition may further include various excipients or carriers, which play a crucial role in enhancing the stability, bioavailability, and overall patient acceptability of the formulation. These excipients are not merely inactive fillers but are specifically chosen to optimize the delivery and effectiveness of the active ingredients, ensuring that the cannabinoids, flavonoids, polyphenols, and alkaloids within the formulation are delivered in a consistent and effective manner. By improving factors such as solubility, absorption, and shelf-life, excipients ensure that the composition remains potent and therapeutically effective over time, while also being easy and comfortable for the patient to use.

In further embodiments, these excipients may include solubilizers, stabilizers, preservatives, and bioavailability enhancers. Solubilizers are essential for ensuring that the active compounds, many of which may be hydrophobic, are properly dissolved and remain in solution, allowing for better absorption by the body. Stabilizers help maintain the integrity of the formulation, preventing the degradation of active ingredients during storage. Preservatives are added to protect the composition from microbial contamination and to extend the shelf life of the product. Bioavailability enhancers, such as certain surfactants or permeation enhancers, are incorporated to improve the absorption of active ingredients, ensuring that the cannabinoids and other compounds reach therapeutic levels in the bloodstream or targeted tissues more efficiently.

In a further embodiment, the composition may also contain emulsifying agents, stabilizing agents, preservative agents, antioxidant agents, solvents, viscosity agents, and emollient agents. Emulsifying agents are particularly important in formulations that combine hydrophilic and lipophilic ingredients, ensuring a homogenous mixture and improving the delivery of active ingredients to the skin or through the gastrointestinal tract. Stabilizing agents may further support the structural integrity of the composition, preventing the separation of ingredients or the breakdown of sensitive compounds like cannabinoids. Preservative agents, such as parabens or benzoates, are used to inhibit microbial growth and maintain product safety, especially in formulations that are stored for extended periods.

Antioxidant agents, such as Vitamin E or other natural compounds, are included to prevent the oxidation of active ingredients, particularly the cannabinoids and polyphenols, which can degrade over time when exposed to air or light. Solvents, such as ethanol or water, may be used to dissolve the active and inactive components, ensuring even distribution within the formulation. Viscosity agents, like cellulose derivatives or glycerin, are added to modify the texture of the product, making it easier to apply or ingest. In topical formulations, emollient agents such as aloe vera, glycerin, or dimethicone can be included to soften and soothe the skin, reducing irritation and enhancing the patient's comfort when using the product.

By incorporating these excipients and carriers, the pharmaceutical composition not only becomes more stable and effective but also more patient-friendly. These agents ensure that the formulation is easy to administer, whether as a capsule or cream, and that it remains effective throughout its intended shelf life. This attention to formulation details helps ensure that the active ingredients are delivered in the most efficient and user-friendly manner possible, maximizing therapeutic outcomes and improving patient compliance.

In one embodiment, the composition can be formulated for multiple administration forms, offering flexibility in its use and allowing it to cater to a wide range of therapeutic needs. This versatility is particularly important in the treatment of conditions like shingles, where the symptoms can vary significantly in severity, duration, and localization. By preparing the composition in various pharmaceutical forms, it ensures that different delivery methods can be employed based on the patient's specific symptoms, preferences, or treatment goals. These forms may include oral capsules for systemic treatment, topical creams for localized symptom relief, sublingual formulations for rapid absorption, or even injectables for acute management of severe cases.

The ability to prepare the composition in different pharmaceutical forms enhances its practicality and accessibility for various patient populations. For instance, oral capsules provide a convenient option for patients who require systemic relief from shingles-related pain, inflammation, and viral activity. Capsules can be formulated with controlled-release mechanisms to ensure a steady delivery of cannabinoids, flavonoids, polyphenols, and alkaloids throughout the day, maintaining consistent therapeutic levels in the bloodstream without the need for frequent dosing. This controlled-release technology can help reduce symptom flare-ups and improve long-term outcomes, particularly in managing chronic pain, such as postherpetic neuralgia.

Topical creams, on the other hand, are ideal for patients who experience localized symptoms such as pain, itching, or rashes. These creams can be applied directly to the affected areas, offering rapid relief by delivering active ingredients to the skin and underlying tissues. Formulating the cream with a slow-release system ensures that the therapeutic effects are prolonged, providing relief over an extended period without the need for frequent reapplication. Furthermore, topical formulations may be designed to include moisturizing or soothing agents, such as aloe vera or glycerin, to enhance skin health and comfort during treatment.

In addition to oral and topical forms, the composition may also be designed for sublingual administration, allowing for faster absorption through the mucous membranes under the tongue. This method bypasses the digestive system, enabling the active ingredients to enter the bloodstream more quickly, which is particularly useful in cases where rapid symptom relief is needed. For more severe cases of shingles, where immediate and potent intervention is required, injectable formulations may be developed. These can be administered in clinical settings, providing fast and direct delivery of the therapeutic compounds to help manage acute outbreaks or severe complications.

Furthermore, the composition can be designed with different controlled-release options to accommodate various therapeutic needs. Immediate-release formulations may be used for acute symptom management, providing fast-acting relief, while sustained-release or extended-release formulations ensure a gradual release of the active ingredients, maintaining therapeutic effects over a longer period. This level of customization allows healthcare providers to tailor treatment plans to the specific needs of each patient, ensuring that they receive the appropriate level of symptom control based on the severity and progression of their shingles symptoms.

By offering multiple administration forms and controlled-release options, the composition provides a comprehensive solution that can be adapted to the diverse therapeutic needs of shingles patients. This flexibility not only improves patient compliance by offering more convenient and effective treatment methods but also ensures that the therapeutic effects are optimized for both immediate and long-term symptom management.

In one embodiment, the phytocannabinoid-based pharmaceutical composition is formulated for multiple routes of administration, each tailored to meet specific therapeutic needs and patient preferences. This multi-faceted approach enhances treatment flexibility, ensuring that patients receive the most appropriate method of administration based on the severity of their symptoms, the target area of treatment, and their individual medical needs. By offering various routes of administration—oral, topical, inhalation, and parenteral—the composition provides clinicians with a broad range of options to optimize patient care, allowing for precise control over dosage, bioavailability, and the timing of therapeutic effects.

Oral administration, in the form of capsules, tablets, or liquids, is one of the most common and convenient methods for delivering phytocannabinoids systemically. This route is particularly useful for patients requiring broad, systemic relief from shingles symptoms, such as widespread pain, inflammation, and immune response regulation. Oral formulations can also be designed with controlled-release mechanisms, providing sustained therapeutic effects over time and reducing the need for frequent dosing. For patients with chronic symptoms like postherpetic neuralgia, these formulations offer long-lasting relief by maintaining steady cannabinoid levels in the bloodstream.

Topical administration, through creams, gels, or ointments, provides a more localized treatment option, allowing cannabinoids to be applied directly to the areas affected by shingles. This route is ideal for targeting skin rashes, pain, and inflammation at the site of the shingles lesions. By applying the phytocannabinoid-based composition topically, patients can achieve fast relief from localized discomfort without the need for systemic exposure. The addition of soothing agents, such as aloe vera or capsaicin, can further enhance the topical formula's effectiveness, alleviating skin irritation and reducing itching, burning, or tingling sensations. Topical formulations can also incorporate slow-release mechanisms to provide prolonged relief, reducing the frequency of application and improving patient compliance.

Inhalation methods, such as vaporizers or inhalers, offer another route of administration that enables rapid onset of action. This approach is particularly effective when immediate relief from pain or inflammation is needed. Phytocannabinoids, when inhaled, are absorbed quickly into the bloodstream through the lungs, bypassing the digestive system and providing near-instant therapeutic effects. This route of administration is especially beneficial for patients experiencing acute flare-ups of shingles pain or those needing quick, potent relief from inflammation. The inhalation route also allows for precise dosage control, enabling patients and clinicians to adjust the amount of cannabinoids administered based on immediate needs.

Parenteral methods, such as intravenous (IV), intramuscular (IM), or subcutaneous injections, are suitable for more severe or acute cases where rapid and direct delivery of the therapeutic compounds is necessary. Parenteral administration allows cannabinoids to be delivered directly into the bloodstream or targeted tissues, providing immediate and highly controlled therapeutic effects. This route is particularly useful in clinical settings where quick intervention is required to manage intense shingles outbreaks or severe complications, such as nerve damage or immune dysfunction. By administering cannabinoids parenterally, clinicians can ensure that the patient receives the full dosage quickly and efficiently, with minimal loss due to metabolic breakdown.

The ability to formulate the phytocannabinoid-based pharmaceutical composition for such a wide range of therapeutic uses and routes of administration provides unparalleled flexibility in patient care. This versatility ensures that treatment can be tailored to individual needs, offering not only control over the dosage but also the timing and localization of therapeutic effects. Whether a patient requires systemic relief, localized treatment, rapid symptom management, or clinical intervention, the availability of multiple administration methods allows for precision in therapy, maximizing the therapeutic benefits of phytocannabinoids while minimizing potential side effects.

In one embodiment, the phytocannabinoid-based pharmaceutical composition is designed for multiple routes of administration and therapeutic applications, providing significant flexibility in treatment and ensuring precise control over dosage and bioavailability to meet the diverse needs of patients. This multi-route approach allows the composition to be customized for individual conditions, ranging from localized symptoms to more systemic issues, and provides options for both rapid and sustained relief. By offering administration routes such as oral, sublingual, topical, inhalation, parenteral, and intravaginal, this formulation ensures that the therapeutic compounds can be delivered in the most effective manner for each patient, maximizing efficacy while minimizing side effects.

Oral administration, through capsules or tablets, provides a convenient and widely accepted method for delivering phytocannabinoids systemically. This method is particularly useful for long-term management of conditions like shingles, where ongoing treatment is necessary to control inflammation, pain, and immune response. Oral formulations can be tailored for immediate or controlled release, allowing patients to maintain consistent therapeutic levels of cannabinoids throughout the day without frequent dosing. The convenience and ease of use make oral administration an ideal choice for many patients.

Sublingual administration, which involves placing a tincture or dissolvable tablet under the tongue, offers faster absorption than oral routes because the active ingredients bypass the digestive system and are absorbed directly into the bloodstream through the mucous membranes. This method is particularly useful for patients who require rapid symptom relief, such as during acute shingles outbreaks or severe pain episodes. Sublingual delivery allows for precise dosing and ensures that the therapeutic effects are felt quickly, making it an excellent option for managing sudden symptom flare-ups.

Topical application allows the composition to be applied directly to the skin, providing localized relief for symptoms such as pain, itching, and inflammation. This method is particularly beneficial for treating shingles lesions or other skin-related conditions. Topical formulations, such as creams, ointments, or gels, enable cannabinoids to penetrate the skin and deliver targeted relief without the need for systemic exposure. This approach is ideal for patients who need to address specific areas of discomfort without affecting the entire body, making it an effective solution for managing localized symptoms with minimal side effects.

Inhalation methods, such as vaporizers or inhalers, provide another option for rapid relief, especially in cases where immediate symptom management is necessary. By inhaling the cannabinoids, patients experience fast absorption through the lungs, resulting in quick onset of therapeutic effects. This method is particularly useful for patients experiencing acute pain or inflammation that requires immediate intervention, as the cannabinoids can reach therapeutic levels in the bloodstream within minutes.

Parenteral administration, including intravenous (IV), intramuscular (IM), or subcutaneous injections, is suitable for severe cases or when precise and immediate dosing is required. This route bypasses the digestive system entirely, delivering the cannabinoids directly into the bloodstream or targeted tissues, which ensures maximal bioavailability and rapid therapeutic effects. Parenteral methods are particularly effective in clinical settings or for patients who need quick intervention for severe symptoms, such as extreme nerve pain or inflammation.

Intravaginal administration offers another specialized route for delivering the phytocannabinoid-based composition, providing targeted relief for conditions that affect the vaginal area. This route allows cannabinoids to be absorbed directly into the local tissues, offering therapeutic benefits such as pain relief and inflammation reduction in conditions involving pelvic or reproductive system issues. The intravaginal route provides localized treatment with minimal systemic effects, making it a highly effective option for patients who need focused relief in these areas.

By designing the phytocannabinoid-based pharmaceutical composition for multiple routes of administration, this approach ensures that treatment can be tailored to the specific needs of each patient, providing both flexibility and precision in managing symptoms. The ability to control dosage and bioavailability across various administration routes allows healthcare providers to optimize therapeutic outcomes while minimizing unwanted side effects, offering a comprehensive solution for a wide range of conditions.

In one embodiment, the phytocannabinoid-based composition is formulated into various oral dosage forms, offering patients a wide range of options for consuming the active compounds in a manner that is convenient, effective, and suited to their specific therapeutic needs. These oral dosage forms include traditional tablets and capsules, which are commonly used for systemic treatment, providing consistent dosing and prolonged relief from conditions like inflammation, pain, and immune dysfunction. Oral capsules and tablets are easy to administer, making them ideal for long-term management of chronic conditions such as postherpetic neuralgia following shingles outbreaks. These forms can be designed with controlled-release mechanisms, ensuring a steady release of cannabinoids over time, thereby reducing the need for frequent dosing and helping to maintain stable therapeutic levels in the bloodstream.

Other oral forms include oral drops and syrups, which provide liquid alternatives for patients who may have difficulty swallowing pills or who prefer more flexible dosing options. Oral drops are particularly useful in pediatric or elderly populations, where dosing precision and ease of administration are important. Syrups and suspensions offer a palatable option for patients who may struggle with the taste of cannabinoids in their pure form. Powders for oral reconstitution can also be formulated, offering patients the flexibility to mix their medication into liquids for easier consumption, while elixirs and emulsions provide additional liquid options that can enhance the bioavailability of cannabinoids, improving absorption and therapeutic effectiveness.

Novel oral dosage forms such as gummies, caramels, sprays, and mouth rinses provide more patient-friendly options that cater to different preferences and lifestyles. Gummies and caramels, for instance, are a discreet and enjoyable way to consume cannabinoids, making adherence to treatment easier, especially for patients who prefer not to take traditional pills. Sprays and mouth rinses allow for easy application, with the cannabinoids being absorbed quickly through the oral mucosa, offering faster relief. These products can also be formulated with flavor enhancements to make the consumption experience more pleasant, further increasing patient compliance.

In one embodiment, sublingual presentations such as capsules, drops, tablets, or films are developed to offer rapid absorption through the mucous membranes, bypassing first-pass metabolism in the liver. This route allows the cannabinoids to enter the bloodstream directly, providing faster therapeutic effects compared to traditional oral forms that must be metabolized through the digestive system. Sublingual drops or films dissolve quickly under the tongue, ensuring that the active ingredients are absorbed efficiently into the bloodstream, making them particularly useful for patients requiring rapid symptom relief, such as those experiencing acute pain flare-ups or sudden episodes of inflammation. Sublingual tablets, capsules, and drops can be precisely dosed, offering both convenience and quick action, which is especially important in managing conditions where fast onset of relief is critical.

By providing such a wide range of oral and sublingual dosage forms, this phytocannabinoid-based composition offers exceptional flexibility in treatment, ensuring that patients can select the most appropriate form for their lifestyle, symptoms, and preferences. Whether requiring fast-acting relief or long-term management, these dosage forms accommodate diverse patient needs, enhancing both the efficacy of the cannabinoids and overall patient adherence to the treatment regimen.

In a preferred embodiment, the pharmaceutical compositions described herein may be formulated for multiple routes of administration, including oral, topical, and injectable forms, to ensure therapeutic flexibility and patient-specific treatment. This multi-route approach allows for precise control over the dosage, bioavailability, and timing of therapeutic effects, providing customized solutions based on the patient's needs and the severity of their symptoms.

For topical administration, the pharmaceutical forms include a wide range of dosage options, such as creams, emulsions, suspensions, lotions, capsules for topical application, solutions, sprays, powders, gels, and ointments. These formulations are designed for rapid absorption through the skin membranes, ensuring that the active cannabinoids are delivered directly to the affected areas for quicker therapeutic effects. This is particularly beneficial for localized symptoms, such as pain, itching, and inflammation associated with skin conditions or nerve pain, as it allows for targeted treatment without the need for systemic exposure.

In further embodiments, the composition may also be provided as injectable solutions or suspensions, designed for intravenous, intramuscular, or subcutaneous administration. These injectable forms offer precise and immediate delivery of cannabinoids, making them suitable for clinical settings where rapid action is required, such as managing acute pain, severe inflammation, or other urgent medical conditions. By bypassing the digestive system and delivering the active ingredients directly into the bloodstream or targeted tissues, injectable forms ensure maximum bioavailability and fast therapeutic onset, providing effective relief in critical situations. This combination of oral, topical, and injectable options allows the composition to accommodate a wide range of therapeutic needs, from routine symptom management to rapid intervention in acute cases.

In some embodiments, these pharmaceutical forms may include immediate-release, sustained-release, or controlled-release options, offering a high degree of flexibility in managing the timing and duration of cannabinoid bioavailability to meet specific therapeutic requirements. Immediate-release formulations are particularly effective in situations where rapid symptom relief is critical, such as during acute shingles outbreaks. These formulations are designed to deliver cannabinoids quickly, ensuring that therapeutic effects are felt soon after administration. This rapid onset can be especially beneficial for managing severe pain, inflammation, or flare-ups, as cannabinoids like CBD and CBG can immediately begin interacting with the body's endocannabinoid system to reduce discomfort and inflammation.

Sustained-release forms, on the other hand, are formulated to release the active cannabinoids gradually over an extended period. This approach helps maintain therapeutic levels in the bloodstream for longer durations, reducing the need for frequent dosing. Sustained-release formulations are ideal for patients who require consistent, long-lasting relief from chronic symptoms, such as the nerve pain and inflammation associated with postherpetic neuralgia following shingles. By providing a slow, steady release of cannabinoids, these formulations help manage symptoms throughout the day, improving the patient's overall quality of life and simplifying treatment regimens by reducing the number of daily doses required.

Controlled-release options offer an even more refined approach by regulating the release rate of cannabinoids with precision, ensuring that steady levels of the active ingredients are maintained in the system for optimal therapeutic management. This type of formulation is especially valuable for chronic conditions where maintaining a consistent cannabinoid concentration is crucial for symptom control. Controlled-release mechanisms can be tailored to release cannabinoids in a way that aligns with the body's natural rhythms or the progression of symptoms, allowing for more predictable and effective management of long-term conditions like chronic nerve pain. These formulations enhance patient adherence to treatment by reducing fluctuations in symptom relief, providing a smoother, more stable therapeutic experience.

By incorporating immediate-release, sustained-release, and controlled-release mechanisms, the composition can be adapted to a wide range of therapeutic needs. Patients experiencing acute shingles symptoms can benefit from rapid relief with immediate-release formulations, while those managing long-term conditions like postherpetic neuralgia or chronic inflammation can achieve consistent symptom control with sustained or controlled-release options. This flexibility in release mechanisms not only enhances the efficacy of the treatment but also improves patient adherence and satisfaction by providing tailored solutions for both short-term relief and long-term management.

In one embodiment, the composition disclosed comprises cannabinoids in the range of 3-12%, flavonoids in the range of 1-5%, and polyphenols in the range of 0.5-3%. In this embodiment, the cannabinoids may include cannabidiol (CBD) at 7% and cannabigerol (CBG) at 1%, with the flavonoid quercetin present at 3% and the polyphenol resveratrol included at 1%. This combination is designed to offer potent anti-inflammatory and analgesic effects, while also providing antioxidant and neuroprotective properties, making it suitable for managing both the acute and chronic symptoms of shingles.

In another embodiment, the composition may contain a cannabinoid concentration of 5%, consisting of 4% CBD and 1% cannabichromene (CBC), while the flavonoid concentration may range from 2-4%, with luteolin at 2.5%. The polyphenol concentration could be 0.8%, with curcumin as the primary polyphenol. This formulation focuses on enhancing immune modulation and reducing nerve pain, while also leveraging curcumin's anti-inflammatory effects to support quicker skin healing in shingles lesions.

In a further embodiment, the composition includes cannabinoids in a 9-11% range, such as 10% CBD and 1% tetrahydrocannabivarin (THCV), paired with flavonoids at 3%, including kaempferol at 2%, and polyphenols at 2%, featuring ellagic acid. The alkaloid component could range from 0.5-2%, with capsaicin included at 0.8% to address pain management through the desensitization of pain receptors. This combination is optimized to provide comprehensive treatment for nerve pain, inflammation, and skin irritation associated with shingles, while promoting faster recovery through the antioxidant and antiviral effects of the included polyphenols and flavonoids.

In an additional embodiment, the cannabinoid component may be at 8%, with a 6% concentration of CBD and 2% cannabinol (CBN), while the flavonoid concentration may be 4%, with apigenin present at 3.5%. The polyphenol concentration could be set at 1.5%, with epigallocatechin gallate (EGCG) as the primary polyphenol. This formulation aims to reduce inflammation, support immune health, and provide neuroprotection, particularly for patients suffering from postherpetic neuralgia. The inclusion of CBN enhances sleep quality and provides sedative effects, while EGCG offers antiviral benefits.

In yet another embodiment, the composition may include cannabinoids in a 4-6% range, with 5% CBD, flavonoids at 2%, such as rutin at 1.5%, and polyphenols at 1%, including resveratrol at 0.8%. Alkaloids may be included at a concentration of 0.5-1%, featuring piperine at 0.7% to enhance the bioavailability of the active ingredients. This formulation is particularly suited for reducing systemic inflammation, enhancing bioavailability, and providing relief from the painful rashes caused by shingles, while also promoting immune resilience.

Finally, in one embodiment, the composition may contain a cannabinoid concentration of 10%, with CBD and CBG at 7% and 3% respectively, flavonoids at 4%, including quercetin at 2%, and polyphenols at 2%, with curcumin as the primary polyphenol. The alkaloid component may range from 1-3%, featuring berberine at 1.5%. This synergistic combination addresses both acute and chronic symptoms, offering enhanced anti-inflammatory effects, pain relief, and immune modulation, making it an ideal formulation for long-term management of shingles and postherpetic neuralgia.

### Kit for the Treatment of Shingles Symptoms

In some embodiments, the method for treating shingles in a human individual comprises administering an effective concentration of the composition, wherein the active principle includes cannabinoids, flavonoids, polyphenols, alkaloids, and stabilizing and solubilizing agents. This method leverages the combined therapeutic properties of these bioactive compounds to provide a comprehensive approach to managing the various symptoms of shingles, such as pain, inflammation, itching, and viral activity, while also promoting overall immune health and skin recovery. The cannabinoids, such as cannabidiol (CBD) and cannabigerol (CBG), are included for their well-documented analgesic, anti-inflammatory, and neuroprotective effects. By interacting with the body's endocannabinoid system, these cannabinoids help modulate pain, reduce inflammation, and support nerve repair, which is particularly important for patients suffering from shingles-related nerve pain or postherpetic neuralgia.

Flavonoids, such as quercetin or luteolin, are incorporated for their anti-inflammatory and antioxidant properties, which work to reduce oxidative stress and promote faster healing of shingles lesions. These compounds also have immunomodulatory effects, helping to regulate the body's immune response and minimize the severity of the viral outbreak. Polyphenols, including resveratrol or curcumin, further enhance the composition's therapeutic profile by providing additional antiviral and anti-inflammatory benefits. Polyphenols are known to inhibit viral replication and protect cells from damage caused by the inflammatory response, supporting both acute relief and long-term recovery from shingles.

Alkaloids, such as capsaicin or berberine, play a critical role in pain management and immune modulation. Capsaicin, for example, desensitizes pain receptors, providing significant relief from the nerve pain and burning sensations commonly experienced during a shingles outbreak. Berberine, on the other hand, supports the immune system's ability to combat viral infections and reduces inflammation, making it a valuable component for controlling both the pain and viral activity associated with shingles.

The inclusion of stabilizing and solubilizing agents ensures that the active ingredients in the composition are effectively absorbed and remain stable over time. Solubilizing agents help dissolve hydrophobic cannabinoids and other bioactive compounds, improving their bioavailability and ensuring that they reach therapeutic levels in the body. Stabilizing agents protect the integrity of the composition, preventing degradation of the active compounds during storage or when exposed to environmental factors like heat or light. Together, these excipients enhance the overall effectiveness of the treatment by ensuring that the bioactive components are delivered in a consistent and stable form, providing reliable and predictable therapeutic outcomes for patients.

By combining these bioactive compounds and excipients, this method offers a multifaceted approach to treating shingles. The composition can be administered in various forms, such as oral capsules, topical creams, or injectable solutions, depending on the patient's needs and the severity of their symptoms. The comprehensive nature of this method addresses not only the immediate symptoms of shingles but also long-term complications like nerve pain and immune dysregulation, making it an effective solution for both acute and chronic management of the condition.

The present disclosure introduces a comprehensive therapeutic kit designed for the treatment and management of shingles, leveraging a cannabinoid-based formulation in conjunction with polyphenols, flavonoids, and alkaloids. This kit offers an integrated solution that addresses the broad spectrum of symptoms associated with shingles, such as acute pain, tingling, itching, and inflammation. By targeting multiple physiological pathways, the kit aims to provide immediate relief during the acute eruptive phase, where painful vesicles and skin rashes dominate, while also managing long-term complications like postherpetic neuralgia. The cannabinoids, particularly cannabidiol (CBD) and cannabigerol (CBG), serve to reduce inflammation, modulate pain, and promote nerve repair, while the polyphenols and flavonoids contribute antioxidant and anti-inflammatory properties, enhancing skin recovery and protecting against further cellular damage.

In addition to its symptom-management capabilities, the therapeutic kit offers significant benefits in terms of immune modulation and antiviral defense. The inclusion of polyphenols, such as resveratrol, and flavonoids like quercetin, helps regulate the immune response, reducing the overactivation of inflammatory pathways that can exacerbate the condition. These compounds, in conjunction with cannabinoids, also provide potential antiviral effects, inhibiting viral replication and shortening the duration of the shingles outbreak. Alkaloids, such as capsaicin, further contribute to the relief of nerve pain, desensitizing affected areas and alleviating discomfort. This multifaceted approach ensures that the therapeutic kit not only addresses the immediate symptoms but also supports long-term recovery and reduces the risk of lingering nerve pain and other chronic issues, providing a holistic solution for patients at all stages of the disease.

In one embodiment, the therapeutic kit for shingles treatment comprises two main products that work in tandem to provide comprehensive care: an oral capsule and a topical cream. The oral capsule is formulated to address systemic symptoms such as persistent pain, tingling, and immune modulation throughout all stages of the disease. This systemic approach ensures that cannabinoids like cannabidiol (CBD) and cannabigerol (CBG), along with polyphenols and flavonoids, can be absorbed into the bloodstream, offering prolonged relief from widespread symptoms. The oral capsule is particularly effective in managing the nerve pain and immune response associated with shingles, helping to reduce inflammation and modulate immune activity, which can limit the intensity and duration of the outbreak. It also helps in preventing long-term complications, such as postherpetic neuralgia, by promoting nerve health and reducing chronic inflammation.

The second product, a topical cream, is developed to provide localized, targeted relief from pain, itching, and inflammation at the site of shingles lesions. Formulated with cannabinoids for their analgesic and anti-inflammatory properties, the cream also includes polyphenols such as resveratrol and flavonoids like quercetin to accelerate skin healing and reduce oxidative stress. This topical application is designed for rapid absorption through the skin, delivering active compounds directly to the affected area to quickly soothe itching and pain while also addressing the inflammation around the lesions. The cream's localized action complements the systemic effects of the oral capsule, providing a dual approach that ensures both immediate relief at the site of irritation and long-lasting systemic control of symptoms. Together, these two products offer a holistic treatment plan that covers both the internal and external manifestations of shingles, giving patients a versatile, effective solution for managing the disease at every stage.

Oral Capsules: The oral capsules included in the therapeutic kit provide a nanoplatform cannabinoid-based formulation specifically designed for daily use in the treatment of shingles. This advanced delivery system ensures that the active ingredients, including cannabinoids, polyphenols, flavonoids, and alkaloids, are encapsulated in nanosized particles to enhance their bioavailability and absorption into the body. By utilizing this nanoplatform technology, the formulation ensures that the cannabinoids—such as cannabidiol (CBD) and cannabigerol (CBG)—are efficiently delivered to the bloodstream, enabling them to exert their therapeutic effects more effectively. Each capsule contains a precise combination of these active cannabinoids, carefully measured to reduce systemic inflammation, alleviate nerve pain, and support immune modulation throughout all stages of the disease. This approach not only helps manage the acute symptoms of shingles but also offers protection against chronic complications, such as postherpetic neuralgia.

Additionally, the oral capsules incorporate polyphenols, such as resveratrol or curcumin, which are known for their antioxidant and antiviral properties. These polyphenols work synergistically with the cannabinoids to inhibit the replication of the varicella-zoster virus (VZV), thereby reducing the duration and severity of the shingles outbreak. The inclusion of flavonoids, such as quercetin, enhances the immune-modulating properties of the formulation, helping to regulate the body's inflammatory response while boosting its natural defense mechanisms. By combining these bioactive compounds, the capsules provide a multi-dimensional therapeutic effect that not only alleviates pain and inflammation but also helps limit viral proliferation. Designed for daily use, these capsules offer sustained relief, supporting long-term recovery and helping to prevent future flare-ups of the virus by maintaining a balanced immune response and reducing oxidative stress throughout the body. This comprehensive approach allows the oral capsules to target both the underlying viral activity and the systemic symptoms of shingles, providing patients with a powerful and effective treatment option.

Active Cannabinoids: Cannabidiol (CBD) and Cannabigerol (CBG) serve as the primary active cannabinoids in the oral capsules designed for shingles treatment. These cannabinoids interact with the body's endocannabinoid system, particularly with CB1 and CB2 receptors, to regulate critical physiological processes, including pain perception, inflammation, and immune response. CBD, well-known for its potent anti-inflammatory and analgesic properties, effectively alleviates symptoms associated with shingles, such as neuralgic pain, itching, and discomfort. Its role in reducing inflammation is essential in controlling the skin rashes and lesions caused by shingles, minimizing the discomfort patients experience during outbreaks. Additionally, CBD has neuroprotective properties that support the repair of damaged nerve tissues, which is crucial for preventing or mitigating the chronic nerve pain associated with postherpetic neuralgia.

Cannabigerol (CBG), though present in smaller amounts compared to CBD, plays an equally important role in the therapeutic profile of the formulation. CBG is a non-psychoactive cannabinoid that also binds to both CB1 and CB2 receptors, offering analgesic and anti-inflammatory effects similar to CBD. However, CBG stands out for its unique neuroprotective qualities, making it particularly effective in addressing nerve damage and the neuralgic pain that often accompanies shingles outbreaks. Furthermore, CBG is known for its antimicrobial and antiviral properties, which contribute to the reduction of varicella-zoster virus (VZV) activity. By inhibiting viral replication and supporting immune function, CBG helps not only to control the severity of the viral infection but also to speed up recovery from herpetic lesions.

Together, CBD and CBG provide a synergistic effect that enhances the overall efficacy of the capsules. Their combined antiviral and immune-modulating properties help reduce the viral load, preventing the varicella-zoster virus from proliferating and worsening the outbreak. This interaction between cannabinoids and the immune system promotes faster recovery from shingles lesions, while also reducing the likelihood of long-term complications like postherpetic neuralgia. By simultaneously targeting pain, inflammation, and viral activity, these active cannabinoids offer a holistic approach to shingles treatment, addressing both the immediate symptoms and the underlying causes of the condition for a more comprehensive recovery.

Flavonoids and Polyphenols: The inclusion of polyphenols, such as resveratrol, and flavonoids, such as quercetin, in the shingles treatment formulation offers additional layers of therapeutic benefits that complement the action of cannabinoids. These bioactive compounds are well-known for their potent antioxidant and anti-inflammatory properties, making them crucial in managing the oxidative stress and inflammation that accompany shingles outbreaks. Oxidative stress, resulting from an overproduction of free radicals during the immune response, can exacerbate skin damage and prolong the healing process of herpetic lesions. Resveratrol and quercetin work synergistically to neutralize these free radicals, protecting skin cells from further damage and accelerating the repair of affected tissues. This antioxidant action promotes faster healing of the shingles rashes and blisters, helping to restore skin integrity more quickly and reduce the risk of scarring or lingering irritation.

In addition to their antioxidant properties, both resveratrol and quercetin possess strong anti-inflammatory effects, which are critical for controlling the inflammation associated with shingles. This inflammation, driven by the immune system's response to the varicella-zoster virus (VZV), often contributes to the pain, redness, and swelling that patients experience during an outbreak. By downregulating the release of pro-inflammatory cytokines, resveratrol and quercetin help to calm the immune response, thereby reducing pain and discomfort. This anti-inflammatory action not only alleviates the acute symptoms of shingles but also plays a pivotal role in preventing chronic complications such as postherpetic neuralgia. By controlling inflammation in the early stages of the disease, these compounds may reduce the likelihood of nerve damage that can lead to persistent neuralgic pain, a common long-term consequence of shingles.

Moreover, resveratrol and quercetin support overall immune function, enhancing the body's ability to fight off the varicella-zoster virus (VZV). Resveratrol, in particular, has demonstrated antiviral properties, including the inhibition of viral replication, which is essential for controlling the spread of the virus within the body. Quercetin, known for its immunomodulatory effects, helps to balance the immune response, ensuring that the body effectively combats the virus without causing excessive inflammation. Together, these polyphenols and flavonoids aid the immune system in reducing the viral load, which shortens the duration of the shingles outbreak and speeds up recovery. Additionally, by alleviating pain and discomfort through their combined anti-inflammatory and antioxidant effects, resveratrol and quercetin contribute to a more comfortable recovery process, improving the patient's overall quality of life during both the acute and post-acute phases of shingles.

The recommended regimen for shingles treatment involves taking one capsule daily, containing a nanoplatform formulation of cannabinoids, polyphenols, and flavonoids, specifically designed to modulate the immune response and reduce systemic inflammation. In one embodiment, the formulation includes cannabidiol (CBD) at 5%, cannabigerol (CBG) at 1%, quercetin at 2%, and resveratrol at 1%. This combination provides comprehensive symptom management, targeting pain, inflammation, and immune modulation over a duration of 4 weeks. This regimen is recommended for patients with moderate shingles symptoms, focusing on alleviating nerve pain, reducing skin inflammation, and promoting faster healing of herpetic lesions.

In another embodiment, the recommended treatment duration may extend to 6 weeks, with a higher concentration of cannabinoids for more severe cases of shingles. The formulation may consist of CBD at 7%, CBG at 2%, quercetin at 3%, and resveratrol at 2%, providing enhanced anti-inflammatory and analgesic effects. This 6-week course is particularly effective for patients experiencing prolonged or severe outbreaks, where controlling inflammation and viral activity is crucial for preventing long-term complications such as postherpetic neuralgia. The extended duration ensures sustained relief and allows the body to fully recover from the viral attack, reducing the chances of chronic nerve pain.

In a different modality, the treatment duration is shortened to 2 weeks for patients experiencing mild symptoms or those in the early stages of a shingles outbreak. In this case, the formulation contains CBD at 3%, CBG at 1%, quercetin at 1%, and resveratrol at 0.5%. This lower-concentration, shorter-duration regimen is designed to quickly modulate the immune response and provide immediate relief from discomfort, reducing the severity of symptoms before they escalate. The 2-week treatment focuses on rapid intervention to prevent the outbreak from progressing into a more severe phase, making it an ideal option for patients who seek early management of the condition.

In a more intensive modality for chronic shingles cases or patients suffering from postherpetic neuralgia, the recommended treatment may extend up to 8 weeks. This extended regimen contains a higher dose of both cannabinoids and polyphenols, with CBD at 8%, CBG at 3%, quercetin at 4%, and resveratrol at 3%. The prolonged use of these ingredients ensures ongoing pain relief, nerve protection, and immune support, helping to manage the chronic nerve pain associated with long-term shingles complications. The 8-week course is particularly beneficial for patients who need sustained support to fully manage the after-effects of a severe outbreak, providing ongoing relief from neuralgic pain and supporting nerve recovery.

For patients requiring a balanced approach between early intervention and ongoing support, a 3-week regimen may be prescribed. In this modality, the formulation includes CBD at 4%, CBG at 1%, quercetin at 1.5%, and resveratrol at 1%. This moderate-dose regimen aims to provide a solid foundation of symptom control, reducing pain and inflammation while allowing the immune system to regain control over the varicella-zoster virus. The 3-week course provides enough time for substantial recovery without committing to a longer-term treatment plan, making it suitable for patients with mild to moderate shingles symptoms who wish to avoid prolonged use of medication.

In another modality, the treatment can be extended to 5 weeks with a focus on stronger immune modulation and antiviral activity. The formulation in this case may contain CBD at 6%, CBG at 2%, quercetin at 2.5%, and resveratrol at 2%. This option is ideal for patients who experience recurring shingles outbreaks or those with weakened immune systems. The 5-week regimen helps to not only manage the immediate symptoms but also strengthen the body's defense mechanisms against future viral reactivation, promoting long-term recovery and prevention.

These varied modalities offer a range of treatment durations and ingredient combinations, ensuring that the therapeutic regimen can be tailored to each patient's specific needs based on the severity and duration of their shingles outbreak. Whether patients require short-term symptom relief or long-term management of chronic complications, these formulations provide flexible options to optimize recovery and improve overall outcomes.

The formulation is optimized using nanoemulsion technology to improve bioavailability and therapeutic efficacy, ensuring that the active compounds are absorbed effectively within the body. By using a nano-sized platform, the cannabinoids and other active ingredients can cross biological barriers more easily, resulting in faster onset of action and sustained therapeutic benefits.

The composition of the capsule for the treatment of shingles comprises a potent combination of cannabinoids, flavonoids, and polyphenols that work synergistically to address pain, inflammation, and viral activity. In one embodiment, the capsule contains 50 mg of cannabidiol (CBD) (8.33%), 20 mg of cannabigerol (CBG) (3.3%), 100 mg of quercetin (16.67%), and 50 mg of resveratrol (8.33%). This formulation is designed to provide comprehensive relief from shingles symptoms, offering anti-inflammatory, analgesic, and antiviral effects. The combination of CBD and CBG modulates the endocannabinoid system, helping to reduce neuralgic pain and inflammation, while quercetin and resveratrol provide antioxidant support, reducing oxidative stress and promoting immune modulation. This specific formulation is well-suited for a 4-week treatment plan aimed at managing moderate shingles outbreaks, reducing pain, and accelerating skin healing.

In another embodiment, the composition may be adjusted to target more severe cases of shingles or chronic complications such as postherpetic neuralgia. For these cases, the capsule formulation could be enhanced to include 75 mg of CBD (12.5%), 30 mg of CBG (5%), 120 mg of quercetin (20%), and 60 mg of resveratrol (10%). This stronger formulation is ideal for patients experiencing more intense or prolonged symptoms, offering increased anti-inflammatory and neuroprotective support. This composition is recommended for a treatment duration of 6-8 weeks, depending on the severity of the condition, to provide sustained relief and prevent long-term nerve damage.

For patients requiring early intervention or those with mild symptoms, a lower-dose formulation may be recommended. In this modality, the capsule could contain 25 mg of CBD (4.16%), 10 mg of CBG (1.66%), 80 mg of quercetin (13.33%), and 40 mg of resveratrol (6.66%). This formulation offers a lighter, yet effective, approach to symptom management, focusing on rapidly reducing inflammation and discomfort in the early stages of a shingles outbreak. A shorter treatment duration of 2-3 weeks is recommended for this lower-dose option, allowing patients to manage symptoms before they escalate while promoting quick recovery without overmedication.

In a different combination designed for patients with recurrent shingles outbreaks, the formulation could include 60 mg of CBD (10%), 25 mg of CBG (4.16%), 110 mg of quercetin (18.33%), and 55 mg of resveratrol (9.16%). This balanced formulation supports both immediate relief from acute symptoms and long-term immune modulation to reduce the likelihood of future outbreaks. A 5-week treatment regimen with this formulation could provide a robust therapeutic effect, helping to manage both the active outbreak and future viral reactivation risks.

For an intensive, short-term regimen, particularly for patients experiencing severe pain and inflammation, the capsule could be formulated with higher concentrations of cannabinoids. In this embodiment, the capsule may contain 100 mg of CBD (16.67%), 40 mg of CBG (6.67%), 150 mg of quercetin (25%), and 75 mg of resveratrol (12.5%). This high-dose formulation is intended for short, intensive use over a period of 1-2 weeks, providing rapid and powerful relief from severe shingles symptoms. This approach is ideal for patients in the acute phase of the disease who require immediate and significant intervention to manage their pain and inflammation.

Lastly, for long-term management of chronic shingles complications, such as postherpetic neuralgia, a maintenance formulation could be developed with 40 mg of CBD (6.67%), 15 mg of CBG (2.5%), 90 mg of quercetin (15%), and 45 mg of resveratrol (7.5%). This lower-maintenance dose is ideal for prolonged use, offering ongoing support for nerve health, inflammation control, and immune modulation. A treatment duration of 8 weeks or more may be recommended for patients who need consistent relief from chronic symptoms, particularly in cases where long-term nerve damage has occurred.

These multiple formulations offer a wide range of options, allowing healthcare providers to tailor the treatment based on the patient's specific needs, whether they require a short-term intervention for acute symptoms or long-term management of chronic complications. By adjusting the concentrations of CBD, CBG, quercetin, and resveratrol, these capsules can be customized to provide the most effective therapy for each stage of shingles and patient profile.

Cream: The second component of the treatment is a topical cream, formulated with a powerful combination of capsaicin and cannabinoids, specifically designed to target localized shingles symptoms such as pain, itching, and inflammation. Capsaicin, a natural alkaloid derived from chili peppers, works by desensitizing pain receptors in the skin, providing significant relief from the burning and tingling sensations commonly associated with shingles. This effect is particularly beneficial for reducing both acute discomfort during the active phase of the outbreak and persistent pain that may develop as postherpetic neuralgia. When combined with cannabinoids like cannabidiol (CBD) and cannabigerol (CBG), the cream offers a multi-faceted approach, addressing both the inflammation around the herpetic lesions and the underlying nerve pain.

The cannabinoids in the cream, including CBD and CBG, enhance the anti-inflammatory and analgesic properties of the formulation. CBD, known for its strong anti-inflammatory effects, helps reduce redness and swelling around the lesions, while CBG contributes additional neuroprotective benefits by supporting nerve health and reducing long-term nerve damage. Together, these cannabinoids work synergistically with capsaicin to calm the affected area, providing rapid and sustained relief from shingles symptoms. The cannabinoids are absorbed through the skin and interact with local cannabinoid receptors, helping to regulate pain signals and reduce the inflammatory response. This makes the cream an ideal solution for patients suffering from both the acute phase of shingles and those at risk of developing chronic pain from postherpetic neuralgia.

The cream is applied generously to the herpetic lesions four times daily for a duration of 4 weeks, ensuring consistent and direct delivery of the active ingredients to the affected area. The frequent application allows the capsaicin and cannabinoids to maintain their therapeutic effects, preventing pain flare-ups and helping to manage itching and irritation. By providing localized relief, the cream minimizes the need for systemic pain medications, offering a targeted treatment that focuses on the areas most impacted by shingles. Additionally, the cream helps to soothe the skin, supporting faster healing of the lesions and reducing the risk of scarring or skin damage.

For patients with more severe symptoms, the duration of the cream application may be extended beyond 4 weeks, particularly in cases where postherpetic neuralgia is present. In such cases, the cream continues to provide long-term pain relief and support for nerve recovery. Moreover, the cream's formulation is designed to be gentle on the skin, with added emollients and moisturizing agents that prevent dryness or irritation, making it suitable for frequent use without causing additional discomfort to the sensitive skin areas affected by shingles. This dual-action approach-combining the immediate effects of capsaicin and the longer-lasting benefits of cannabinoids-offers a comprehensive solution to managing the localized symptoms of shingles while also supporting long-term skin and nerve health.

Capsaicin: The inclusion of capsaicin in the cream formulation plays a critical role in providing targeted relief from the pain and discomfort associated with shingles, particularly the burning, tingling, and heightened sensitivity that many patients experience. Capsaicin works by interacting with transient receptor potential vanilloid 1 (TRPV1) receptors, which are responsible for transmitting pain signals from the skin to the nervous system. By desensitizing these receptors, capsaicin effectively reduces the sensation of pain, making it especially useful in managing the acute discomfort caused by the shingles rash. This desensitization helps alleviate the sharp, burning pain that accompanies the eruption of herpetic lesions, offering patients a significant reduction in pain without relying on systemic analgesics.

In addition to its potent analgesic properties, capsaicin contributes to the anti-inflammatory effects of the cream. Inflammation is a key factor in both the acute phase of shingles and the development of chronic pain conditions like postherpetic neuralgia. By reducing inflammation in the affected skin areas, capsaicin not only minimizes immediate discomfort but also plays a role in preventing long-term complications. Capsaicin helps to lower the release of pro-inflammatory cytokines, which can exacerbate pain and prolong the healing process. This anti-inflammatory action promotes faster healing of herpetic lesions, allowing the skin to recover more quickly and reducing the risk of scarring or lingering sensitivity.

Furthermore, capsaicin has been shown to decrease hypersensitivity in areas affected by nerve damage, making it a valuable component for preventing and managing postherpetic neuralgia. Postherpetic neuralgia occurs when nerve fibers are damaged during the shingles infection, leading to chronic pain that persists long after the skin has healed. Capsaicin's ability to reduce hypersensitivity in these damaged nerves can help lower the risk of developing this condition by interrupting the cycle of pain signal transmission early in the treatment process. Over time, consistent application of capsaicin helps to “quiet” these overactive pain pathways, reducing the likelihood of long-term neuralgic pain and improving the patient's overall recovery experience.

By combining its analgesic, anti-inflammatory, and desensitizing effects, capsaicin provides a multifaceted approach to managing shingles symptoms. It offers both immediate relief from the painful rash and long-term benefits in reducing the risk of postherpetic neuralgia. As a key ingredient in the cream, capsaicin ensures that patients receive targeted, localized relief from their most bothersome symptoms, supporting a more comfortable recovery and preventing the progression of shingles into chronic pain conditions.

Aloe Vera-Based Formulation: The cream utilizes aloe vera as a primary base, capitalizing on its well-documented soothing, moisturizing, and anti-inflammatory properties to enhance the overall therapeutic efficacy of the formulation. Aloe vera has long been recognized for its ability to calm irritated skin, making it an ideal vehicle for delivering the active ingredients, particularly in the context of shingles, where patients experience significant skin irritation, itching, and inflammation. Its natural cooling effect immediately soothes the burning and itching sensations associated with herpetic lesions, providing instant relief from discomfort while creating an environment conducive to healing.

One of the key benefits of using an aloe vera base is its ability to enhance the absorption of active cannabinoids like cannabidiol (CBD) and cannabigerol (CBG). Aloe vera's unique composition, rich in polysaccharides and glycoproteins, facilitates deeper penetration of these active compounds into the skin layers. By increasing the bioavailability of cannabinoids at the site of application, aloe vera ensures that CBD and CBG are efficiently delivered to the affected areas, where they can interact with local cannabinoid receptors. This enhanced absorption allows the cannabinoids to exert their full anti-inflammatory, analgesic, and neuroprotective effects, providing relief from pain, reducing inflammation, and supporting nerve health. The synergy between aloe vera and cannabinoids makes the formulation more effective at managing the acute and chronic symptoms of shingles, such as pain and postherpetic neuralgia.

Additionally, aloe vera contributes significantly to skin recovery by accelerating the healing process of shingles lesions. Its natural ability to stimulate fibroblast activity enhances collagen production, which is essential for repairing damaged skin tissues. This not only helps the herpetic blisters heal faster but also minimizes the risk of scarring. Aloe vera's hydrating properties further ensure that the skin remains moisturized, preventing the dryness and flakiness that often accompany healing rashes. By keeping the skin supple and hydrated, aloe vera reduces the discomfort associated with tight, inflamed skin and promotes smoother, healthier skin recovery. Its anti-inflammatory properties also complement the cannabinoids in reducing redness, swelling, and irritation, offering a comprehensive treatment for the skin-related symptoms of shingles.

Furthermore, aloe vera's natural antioxidant content helps protect the skin from further oxidative damage, which can exacerbate inflammation and delay healing. By neutralizing free radicals, aloe vera supports the overall health of the skin while mitigating the inflammatory response triggered by the varicella-zoster virus. The combined cooling, moisturizing, and healing effects of aloe vera make it an essential component of the cream, ensuring that patients receive immediate relief from itching and inflammation while promoting faster and more comfortable skin recovery. This holistic approach not only addresses the visible symptoms of shingles but also works to prevent long-term skin damage and discomfort.

The composition of the cream for the treatment of shingles includes carefully selected ingredients that work together to provide relief from pain, inflammation, and skin irritation while promoting faster healing. In one embodiment, the formulation contains 12.00 mg of Aloe Vera Gel (30%), 0.91 mg of cannabidiol (CBD) (2.27%), 0.60 mg of cannabigerol (CBG) (1.51%), and 0.09 mg of capsaicin (0.23%). This combination offers a balanced approach to soothing shingles lesions, with aloe vera acting as a hydrating and anti-inflammatory base that enhances the absorption of cannabinoids. CBD and CBG deliver potent anti-inflammatory and analgesic effects, while capsaicin helps to desensitize pain receptors, providing immediate relief from the burning and tingling sensations commonly associated with shingles.

In a more concentrated formulation, the cream may include 10.00 mg of Aloe Vera Gel (25%), 1.50 mg of cannabidiol (CBD) (3.75%), 1.00 mg of cannabigerol (CBG) (2.50%), and 0.12 mg of capsaicin (0.30%). This version of the cream increases the concentrations of both CBD and CBG, making it more suitable for patients experiencing severe pain and inflammation. The higher cannabinoid content enhances the anti-inflammatory effects, targeting nerve pain while providing additional support for skin healing. This formula is particularly effective for patients with more intense symptoms, such as postherpetic neuralgia or significant nerve damage caused by shingles.

Another possible formulation might lower the concentration of cannabinoids while focusing more on the soothing and hydrating effects of aloe vera. This combination could contain 15.00 mg of Aloe Vera Gel (35%), 0.70 mg of cannabidiol (CBD) (1.75%), 0.40 mg of cannabigerol (CBG) (1.00%), and 0.08 mg of capsaicin (0.20%). This lower concentration of cannabinoids would still offer anti-inflammatory and pain-relieving benefits but would be more appropriate for patients with milder symptoms or those in the early stages of a shingles outbreak. The increased aloe vera concentration enhances the cream's moisturizing effects, keeping the skin hydrated and soothing irritation.

For patients seeking rapid pain relief with a higher emphasis on capsaicin, a formulation might include 8.00 mg of Aloe Vera Gel (20%), 0.90 mg of cannabidiol (CBD) (2.25%), 0.50 mg of cannabigerol (CBG) (1.25%), and 0.15 mg of capsaicin (0.37%). The increased capsaicin content in this version provides stronger desensitization of pain receptors, which is particularly beneficial for patients suffering from acute nerve pain. The cannabinoids and aloe vera continue to support healing and reduce inflammation while the capsaicin addresses the immediate pain associated with shingles.

Alternatively, a formulation designed for long-term use and prevention of postherpetic neuralgia may include 12.00 mg of Aloe Vera Gel (30%), 1.00 mg of cannabidiol (CBD) (2.50%), 0.70 mg of cannabigerol (CBG) (1.75%), and 0.10 mg of capsaicin (0.25%). This formulation provides sustained anti-inflammatory and analgesic effects, making it suitable for extended application in patients dealing with chronic symptoms or nerve damage. The balanced cannabinoid content helps manage ongoing inflammation and nerve pain, while the capsaicin reduces sensitivity over time, helping prevent the development of chronic pain.

In some embodiments, the purpose of the kit is to provide both systemic and localized relief of shingles symptoms, offering a natural and holistic approach to improving the quality of life for individuals suffering from this condition. The comprehensive nature of the kit is designed to target the various symptoms of shingles, including pain, inflammation, itching, and discomfort caused by herpetic lesions, while also supporting the body's immune response and recovery. By incorporating both oral and topical treatments, the kit aims to deliver a balanced and multifaceted solution to managing the acute and long-term complications of shingles, such as postherpetic neuralgia.

In a preferred embodiment, the disclosure comprises a kit for the comprehensive treatment of shingles, consisting of two primary products: capsules and a topical cream. The capsules provide systemic support by modulating the immune response, reducing inflammation throughout the body, and addressing widespread pain. These capsules typically include a combination of cannabinoids, polyphenols, flavonoids, and other active ingredients known for their immune-modulating, antiviral, and anti-inflammatory properties. The systemic treatment targets the underlying viral activity of the varicella-zoster virus (VZV) while providing relief from neuralgic pain and preventing the onset of chronic complications. The capsules also support the body's natural healing processes, helping to regulate immune function and reduce the risk of prolonged inflammation and nerve damage.

The topical cream, on the other hand, is formulated to offer localized relief from the most immediate and uncomfortable symptoms of shingles, such as pain, itching, and inflammation at the site of the herpetic lesions. By using a combination of ingredients like cannabinoids (CBD, CBG), capsaicin, and aloe vera, the cream works directly on the affected areas to soothe irritation, reduce redness, and accelerate skin recovery. Capsaicin provides targeted pain relief by desensitizing pain receptors, while cannabinoids help reduce localized inflammation and promote skin healing. The aloe vera base ensures that the cream is gentle and hydrating, offering a cooling effect that provides additional comfort to irritated skin.

In one embodiment, the kit is designed specifically for the comprehensive treatment of shingles symptoms, where the oral treatment (capsules) works synergistically with the topical treatment (cream) to address both systemic and localized aspects of the condition. The capsules offer long-lasting systemic relief by managing the immune system and reducing viral activity, while the cream provides fast-acting relief at the site of discomfort, addressing pain, itching, and inflammation more directly.

Together, these treatments ensure that the patient receives both immediate relief from acute symptoms and longer-term support to prevent chronic issues, such as postherpetic neuralgia, which can persist long after the initial shingles outbreak.

Aspects of the disclosure refer to a composition for the treatment of shingles that includes: a) an oral treatment designed to provide systemic support through immune modulation, antiviral action, and inflammation control; and b) a topical treatment designed to relieve localized symptoms by reducing pain, inflammation, and irritation at the site of shingles lesions. This dual approach ensures that all facets of the disease are addressed comprehensively, providing patients with a natural and effective way to manage both the acute and long-term symptoms of shingles. This holistic treatment method helps improve overall quality of life during the course of the condition and supports faster, more complete recovery.

Product 1—capsules: is a composition comprising cannabinoids, flavonoids and polyphenols, specifically: The oral treatment comprises capsules. The capsules comprise: a) an active principle comprising at least two cannabinoids; b) an active principle comprising at least one polyphenol, one flavonoid, or combinations thereof c) a stabilizing solubilizing agent.

In some embodiments, the method for treating shingles in a human individual comprises administering an effective concentration of the composition, wherein the active principle comprises cannabinoids, flavonoids, polyphenols, alkaloids, and stabilizing solubilizing agents. In one further embodiment, the active principle comprises (Cannabidiol) CBD and Cannabigerol (CBG).

In a further embodiment, the composition comprises a second active principle comprising one flavonoid, one polyphenol, and one alkaloid. In a preferred embodiment the flavonoids, polyphenols, and alkaloids are quercetin, resveratrol, and capsaicin, respectively. In a further preferred embodiment, the capsule includes resveratrol and quercetin in addition to the cannabinoids.

In one embodiment, the composition includes a combination of cannabinoids, flavonoids, and polyphenols at concentrations specifically designed to provide a potent and multifaceted approach to the treatment of shingles. For example, the composition may include CBD at a concentration ranging from 6% to 9%, and CBG at a concentration of 2% to 4%, with a preferred embodiment comprising CBD at 8.33% and CBG at 3.33%. These cannabinoids work synergistically to modulate pain and inflammation, making them especially effective in managing the discomfort and nerve pain associated with shingles. The anti-inflammatory properties of CBD help reduce systemic inflammation, while CBG offers neuroprotective benefits, helping to mitigate nerve damage and reduce the risk of postherpetic neuralgia.

In another embodiment, the composition also includes flavonoids and polyphenols, such as quercetin and resveratrol, at a concentration ranging from 18% to 25% per capsule. The flavonoid quercetin may be present at a concentration between 12% and 17%, and the polyphenol resveratrol at a concentration ranging from 6% to 9%. These bioactive compounds complement the cannabinoids by providing strong antioxidant and antiviral properties. Quercetin enhances the immune system's ability to combat viral infections while also reducing the inflammatory response that contributes to pain and discomfort. Resveratrol, known for its antiviral properties, helps inhibit the replication of the varicella-zoster virus, reducing the severity and duration of the shingles outbreak. Together, these ingredients form a powerful combination that targets both the symptoms and underlying viral activity associated with shingles.

In a further embodiment, the composition is administered orally in the form of a single capsule, each containing 50 mg of CBD (8.33%), 20 mg of CBG (3.33%), 100 mg of quercetin (16.67%), and 50 mg of resveratrol (8.33%). This formulation is designed to deliver comprehensive symptom relief by addressing pain, inflammation, and viral activity simultaneously. The inclusion of high concentrations of quercetin and resveratrol enhances the overall efficacy of the treatment by supporting immune modulation and promoting faster recovery from shingles lesions. This combination also helps reduce the likelihood of developing chronic complications, such as postherpetic neuralgia, by protecting nerve cells and reducing oxidative stress.

In other possible formulations, the composition may vary the concentrations of these active ingredients to suit different patient needs. For example, in one embodiment, the composition may include 60 mg of CBD (9%) and 25 mg of CBG (3.75%), combined with 110 mg of quercetin (18.33%) and 55 mg of resveratrol (9.16%). This formulation offers a stronger dose for patients with more severe symptoms or those who require more aggressive intervention to control the viral infection and manage chronic pain. The higher concentrations of cannabinoids and polyphenols in this formulation provide enhanced pain relief and anti-inflammatory effects, making it particularly suitable for long-term management of shingles and its complications.

In yet another embodiment, the composition may be formulated with lower concentrations of active ingredients for patients with mild symptoms or early-stage shingles. For example, the capsule may contain 40 mg of CBD (6.67%), 15 mg of CBG (2.5%), 80 mg of quercetin (13.33%), and 40 mg of resveratrol (6.66%). This lighter formulation still provides effective symptom relief while offering a more moderate dose that may be appropriate for early intervention or patients with milder outbreaks. The focus on early treatment with lower doses can help prevent the progression of the condition and reduce the risk of severe symptoms or long-term complications.

In further embodiments, the composition may also incorporate additional cannabinoids, such as tetrahydrocannabivarin (THCV) or cannabinol (CBN), to enhance the therapeutic effects. For example, a formulation might include 50 mg of CBD (8.33%), 20 mg of CBG (3.33%), 90 mg of quercetin (15%), 45 mg of resveratrol (7.5%), and 10 mg of THCV (1.67%). THCV is known for its ability to modulate immune response and provide additional anti-inflammatory and analgesic benefits, making it a valuable addition for more complex cases of shingles. This combination would be particularly useful for patients experiencing severe immune dysregulation or heightened inflammation, as THCV can help balance immune function while supporting nerve health.

By providing these multiple combinations of cannabinoids, flavonoids, and polyphenols in various concentrations, the composition can be tailored to meet the specific needs of patients at different stages of shingles. Whether patients require early intervention, long-term management, or enhanced antiviral and pain-relief effects, these formulations offer a customizable approach to treatment that ensures optimal therapeutic outcomes. The flexibility in dosage and ingredient combinations allows healthcare providers to fine-tune the treatment to achieve the best possible results for each individual patient.

In one further embodiment, the composition includes flavonoids and polyphenols at a concentration ranging from 18% to 25% per capsule. In a further embodiment, the flavonoids include quercetin, while the polyphenols include resveratrol. In one embodiment, the composition includes resveratrol at a concentration ranging from 6.0% to 9.0% per capsule. In another embodiment, the composition includes quercetin at a concentration ranging from 12% to 17% per capsule. In further embodiments, the composition is administered orally in the form of 1 capsule, each containing a concentration of: CBD: 8.33%, CBG: 3.33%, Quercetin: 16.67%, Resveratrol: 8.33%.

In one embodiment, the composition includes cannabinoids at a concentration ranging from Capsules: CBD: 6%-9%, CBG 2%-4%. In one preferred embodiment, the composition comprises the following concentrations: CBD at 8.33%, and CBG at 3.33%. In one non-limiting embodiment, this composition for shingles care contains the following active ingredients per dosage: CBD-50 mg; CBG-20 mg. In further embodiments, the composition will be administered orally in the form of a capsule, each containing a concentration of CBD: 50 mg, CBG: 20 mg quercetin: 100 mg; and resveratrol: 50 mg.

In one embodiment, the cannabinoid pharmaceutical composition may also include other therapeutic agents such as analgesics, anti-inflammatories, and antipruritics to further enhance its efficacy in treating shingles and providing comprehensive symptom relief. By incorporating additional therapeutic agents, the composition is designed to offer a multi-targeted approach, addressing not only the pain and inflammation caused by shingles but also the itching and discomfort associated with herpetic lesions.

The inclusion of analgesics, such as acetaminophen or ibuprofen, enhances the pain-relieving effects of the cannabinoids like cannabidiol (CBD) and cannabigerol (CBG). While cannabinoids already offer significant analgesic properties through their interaction with the body's endocannabinoid system, combining them with traditional painkillers can provide more immediate relief, especially in cases of acute shingles pain or postherpetic neuralgia. This combination allows for a reduction in pain severity and duration, helping to manage both localized nerve pain and widespread discomfort, thus improving patient quality of life during the active phase of the condition.

In addition to analgesics, the composition may include anti-inflammatory agents such as non-steroidal anti-inflammatory drugs (NSAIDs) or corticosteroids. These agents work alongside cannabinoids to significantly reduce inflammation, both systemically and locally at the site of the shingles lesions. Inflammation plays a central role in the pain and discomfort associated with shingles, and by combining the natural anti-inflammatory properties of cannabinoids with these therapeutic agents, the composition offers a more potent solution for controlling swelling, redness, and irritation. This dual action helps prevent further tissue damage and promotes faster healing of the lesions, while also reducing the likelihood of long-term nerve damage that can lead to chronic pain conditions like postherpetic neuralgia.

The antipruritic (anti-itch) agents, such as menthol, calamine, or diphenhydramine, may also be added to the formulation to address the severe itching that often accompanies shingles outbreaks. The antipruritic ingredients work by soothing the skin and calming the itch response, which can be a major source of discomfort for patients. When combined with cannabinoids, which also offer localized anti-inflammatory and calming effects, the antipruritics can help reduce the urge to scratch the lesions, thereby preventing further irritation, infection, or scarring. This not only enhances patient comfort but also supports the healing process by keeping the skin calm and intact.

Moreover, the composition's synergy between cannabinoids and these additional therapeutic agents provides a more tailored approach to shingles care, allowing for both short-term relief of acute symptoms and long-term management of chronic issues. The cannabinoids act as the base of the treatment, modulating pain and inflammation, while the analgesics, anti-inflammatories, and antipruritics act as supportive agents that target specific symptoms more aggressively. This combination approach ensures that patients receive a well-rounded treatment that addresses the complex nature of shingles, from pain and itching to inflammation and viral activity.

In some embodiments, these additional therapeutic agents are formulated in precise ratios to minimize side effects while maximizing efficacy. For example, the amount of NSAIDs or corticosteroids could be carefully controlled to prevent gastrointestinal or other side effects, while still providing adequate anti-inflammatory action. Similarly, the analgesics would be included at levels that complement the cannabinoid's pain-relief mechanism without causing over-sedation or dependence. The goal of incorporating these agents is to amplify the overall therapeutic impact of the composition without compromising patient safety, offering an effective and well-balanced treatment for shingles symptoms.

By including analgesics, anti-inflammatories, and antipruritics in the cannabinoid pharmaceutical composition, the treatment becomes more versatile and effective at managing the full range of shingles symptoms. This approach not only provides rapid relief from acute pain, itching, and inflammation but also supports faster recovery and helps prevent long-term complications, offering patients a more comfortable and comprehensive path to healing.

In one embodiment, the cannabinoid pharmaceutical composition not only includes therapeutic agents such as cannabinoids, polyphenols, and flavonoids but also incorporates stabilizing solubilizing agents, antioxidants, preservatives, and lipid agents to enhance the formulation's efficacy and shelf stability. These additional components are carefully selected to ensure that the active ingredients are delivered effectively, maintaining their potency and bioavailability over time.

The stabilizing solubilizing agents play a critical role in improving the solubility and stability of the cannabinoids, which are naturally lipophilic and can be difficult for the body to absorb. In further embodiments, the stabilizing solubilizing agents may include a combination of Polyethylene Glycol, Polysorbate, Sorbitan, Sodium Lauryl Sulfate, Lecithin, and Poloxamers. These agents work together to ensure that the cannabinoids, polyphenols, and flavonoids are solubilized efficiently, allowing them to be absorbed into the bloodstream more effectively. In one preferred embodiment, the solubilizing agent is a combination of Sorbitan, Polysorbate, and Polyethylene Glycol, which optimizes the delivery of the active ingredients by enhancing their bioavailability and ensuring consistent therapeutic effects.

In addition to solubilizing agents, the composition includes antioxidants to protect the active ingredients from oxidative degradation. Antioxidants are essential for maintaining the stability and potency of the cannabinoids, polyphenols, and flavonoids during storage and throughout their shelf life. In further embodiments, the antioxidants may comprise but are not limited to Vitamin E, EDTA, Vitamin C, and combinations thereof. These antioxidants help prevent the oxidation of the formulation, ensuring that the therapeutic agents retain their efficacy. In one preferred embodiment, the antioxidant used is Vitamin E, which not only acts as a preservative but also offers additional skin benefits, supporting the healing of shingles lesions and reducing inflammation.

Preservatives are also included in the formulation to protect it from microbial contamination and extend its shelf life. In further embodiments, the preservatives may include but are not limited to Propylparaben, Methylparaben, Benzoic Acid, Sodium Benzoate, Potassium Sorbate, and combinations thereof. These preservatives work to inhibit the growth of bacteria, fungi, and other microorganisms that could compromise the safety and effectiveness of the product. In one preferred embodiment, the preservative is a combination of Propylparaben and Methylparaben, which is widely recognized for its efficacy in preventing microbial contamination while being gentle on the skin.

The lipid agents in the formulation are another crucial component, as they aid in the absorption of the lipophilic cannabinoids and provide additional moisturizing benefits for the skin. In further embodiments, the lipid agents may comprise but are not limited to Medium Chain Triglycerides (MCT oil), Mineral Oil, Olive oil, and combinations thereof. These lipid agents help improve the delivery of the active ingredients by creating an emulsion that enhances absorption through the skin and mucosal membranes. In one preferred embodiment, the lipid agent is Medium Chain Triglycerides (MCT oil), which is known for its ability to enhance the bioavailability of cannabinoids and other active ingredients while providing a soothing, moisturizing effect on the skin.

By incorporating stabilizing solubilizing agents, antioxidants, preservatives, and lipid agents, the cannabinoid pharmaceutical composition ensures that the active ingredients are delivered effectively and remain stable throughout their shelf life. This combination of components not only enhances the bioavailability and therapeutic efficacy of the cannabinoids, polyphenols, and flavonoids but also provides a formulation that is safe, stable, and easy to administer. The use of these agents ensures that the composition maintains its potency, offers consistent relief from shingles symptoms, and promotes faster recovery, all while being gentle on the skin and minimizing the risk of irritation or adverse reactions.

In further embodiments, the emulsifying and solubilizing system within the composition is carefully designed to optimize the bioavailability and stability of the active ingredients, such as cannabinoids, polyphenols, and flavonoids. The emulsifying agents used in this system serve to blend the lipophilic cannabinoids with water-based components, creating a stable emulsion that enhances the absorption of these active compounds when administered orally. The emulsifying agents may comprise but are not limited to Propylene Glycol, Sorbitan, Polysorbate, Lecithin, Sodium Lauryl Sulfate, and Poloxamers. These agents play a key role in ensuring that the cannabinoids (such as CBD and CBG) and other active ingredients are solubilized effectively, enabling them to be absorbed more efficiently by the digestive system.

Propylene Glycol, in particular, acts as both a solvent and an emulsifier, helping to dissolve cannabinoids and facilitate their incorporation into the aqueous components of the capsule. Sorbitan and Polysorbate work in tandem to stabilize the emulsion, ensuring that the mixture remains homogenous and preventing separation of the oil and water phases. Lecithin, a naturally occurring emulsifier, also plays a critical role in enhancing the absorption of lipophilic ingredients by reducing the surface tension between oil and water molecules. Sodium Lauryl Sulfate and Poloxamers further assist in the emulsification process, helping to maintain the stability of the capsule and ensuring the efficient delivery of active ingredients into the bloodstream.

The presence of stabilizing and solubilizing agents within the composition, in combination with emulsifying agents, ensures that the active compounds remain in solution, maintaining their bioavailability and potency over time. These agents prevent the active ingredients from precipitating out of the mixture, ensuring that each dose delivers the intended therapeutic benefits. The stabilizing solubilizing agents protect the cannabinoids and other active compounds from degradation, preserving their efficacy throughout the product's shelf life.

The concentration of stabilizing solubilizing agents, emulsifying agents, and preservatives within the composition ranges from 12% to 17% per capsule. This specific concentration range has been optimized to balance the stability of the formulation with the need for efficient absorption of the active ingredients. The inclusion of preservatives is essential to maintaining the integrity of the composition by preventing microbial growth and protecting against contamination. Common preservatives used in this formulation may include but are not limited to Propylparaben, Methylparaben, Benzoic Acid, Sodium Benzoate, and Potassium Sorbate. These preservatives help extend the shelf life of the product and ensure its safety for consumption, while being present in concentrations that do not interfere with the therapeutic effects of the cannabinoids and other active ingredients.

By utilizing a carefully selected emulsifying and solubilizing system, the composition ensures that the active ingredients are effectively delivered and absorbed, maximizing their therapeutic potential in the treatment of shingles. The inclusion of emulsifiers such as Propylene Glycol, Sorbitan, Polysorbate, Lecithin, Sodium Lauryl Sulfate, and Poloxamers, along with stabilizing agents and preservatives, provides a stable and reliable formulation that can be safely and consistently administered to patients. This system enhances the bioavailability of cannabinoids, ensuring that they reach therapeutic levels in the bloodstream to modulate inflammation, manage pain, and support recovery from shingles.

It should be noted that the properties described in various embodiments herein could be combined in different ways to create additional embodiments of the present disclosure. Further aspects and features of the disclosure will be evident to those skilled in the art.

In some embodiments, the composition includes cannabinoids, flavonoids and polyphenols as active principles, designed to offer a multi-faceted approach to treating conditions such as shingles. These active compounds work synergistically to address inflammation, pain, immune modulation, and support overall recovery. The cannabinoids, such as cannabidiol (CBD) and cannabigerol (CBG), are central to the formulation due to their known anti-inflammatory, analgesic, and neuroprotective properties. Flavonoids like quercetin and polyphenols such as resveratrol add further antioxidant and immune-regulating effects, making the composition especially effective in promoting skin healing and protecting against viral replication.

In a further embodiment, the composition includes cannabinoids at concentrations ranging from 8% to 12%, polyphenols ranging from 6% to 9%, and flavonoids ranging from 12% to 17% per capsule. This range allows flexibility in dosing, tailored to the patient's specific needs and the severity of their condition. For example, higher concentrations of cannabinoids may be appropriate for individuals with more severe inflammation and pain, while the inclusion of higher amounts of quercetin or resveratrol can offer enhanced immune support for those dealing with recurrent viral outbreaks or chronic postherpetic neuralgia.

In a preferred embodiment, the composition includes cannabinoids such as CBD and CBG, alongside flavonoids and polyphenols such as quercetin and resveratrol. These compounds offer a balanced and targeted approach, with CBD working to alleviate pain and reduce inflammation, and CBG providing additional neuroprotective effects that help protect nerve cells from damage, particularly in the context of nerve pain. Quercetin contributes strong antioxidant effects, helping reduce oxidative stress and inflammation, while resveratrol offers antiviral properties, making the composition effective not only for symptom management but also in controlling viral activity.

In a further preferred embodiment, the composition is formulated with specific concentrations of these active principles, such as CBD at 8.33%, CBG at 3.33%, quercetin at 16.67%, and resveratrol at 8.33%. This precise formulation provides a balanced therapeutic effect, optimizing the synergy between cannabinoids, flavonoids, and polyphenols to provide comprehensive relief. CBD and CBG target both systemic and localized pain and inflammation, while quercetin and resveratrol provide additional immune modulation and antiviral activity. This makes the composition ideal for managing both the acute symptoms of shingles and reducing the likelihood of chronic complications like postherpetic neuralgia.

In one embodiment, a variation of the composition may include CBD at 9%, CBG at 4%, quercetin at 15%, and resveratrol at 6%. This formulation provides a higher concentration of cannabinoids, designed for more intense pain relief and inflammation control, while maintaining substantial levels of flavonoids and polyphenols to support immune function and promote faster recovery of herpetic lesions. The increased CBG content also enhances neuroprotection, making this combination particularly suited for patients suffering from severe neuralgic pain.

In another embodiment, the composition may include 8% CBD, 2% CBG, 12% quercetin, and 9% resveratrol. This balance is ideal for patients who require moderate pain relief but need stronger antiviral support. The increased concentration of resveratrol helps inhibit the replication of the varicella-zoster virus, making the formulation effective for managing outbreaks while providing sufficient immune support and reducing inflammation.

A different formulation might include 10% CBD, 3% CBG, 14% quercetin, and 7% resveratrol. This configuration is designed to offer a mid-range solution, combining effective anti-inflammatory and pain-relief properties with immune-enhancing and antiviral effects. The balance of CBD and quercetin in this formulation ensures that inflammation is reduced effectively, while resveratrol continues to support the body's defense against viral replication.

For a more aggressive approach to managing severe shingles symptoms, the composition may include 11% CBD, 4% CBG, 17% quercetin, and 6% resveratrol. This high-concentration formulation is designed for patients dealing with significant inflammation, pain, and viral activity. The increased levels of CBD and CBG provide potent anti-inflammatory and analgesic effects, while quercetin and resveratrol offer enhanced immune support and antiviral properties. This formulation is particularly useful in cases of recurrent outbreaks or for patients who are at higher risk for chronic pain conditions like postherpetic neuralgia.

In yet another embodiment, the composition may include 6% CBD, 3% CBG, 13% quercetin, and 9% resveratrol. This lower-concentration cannabinoid formulation focuses on providing strong antioxidant and immune-boosting benefits while still offering moderate pain relief and inflammation control. The increased levels of quercetin and resveratrol make this composition highly effective for patients who require significant immune support, such as those prone to viral infections or those dealing with lingering post-shingles symptoms like fatigue or mild neuralgia.

In all embodiments, the bioavailability of these active ingredients is enhanced through the use of stabilizing and solubilizing agents such as Polyethylene Glycol, Polysorbate, Sorbitan, Lecithin, Sodium Lauryl Sulfate, and Poloxamers, which ensure that the cannabinoids, flavonoids, and polyphenols are delivered efficiently to the bloodstream. These agents ensure the solubility and stability of the active compounds, preserving their potency and improving absorption. In particular, a preferred embodiment may use a combination of Sorbitan, Polysorbate, and Polyethylene Glycol to enhance the solubility of cannabinoids, while preserving their bioactive properties throughout the digestion process.

Additionally, the composition may include antioxidants like Vitamin E and preservatives such as Propylparaben and Methylparaben to protect the formulation from oxidative degradation and microbial contamination. This ensures that the active ingredients remain stable and effective throughout their shelf life.

By offering these varied combinations of active ingredients and supporting agents, the composition can be tailored to meet the specific needs of different patients, ranging from those seeking early intervention to those requiring long-term management of chronic symptoms.

In another embodiment, the composition not only includes cannabinoids, flavonoids, and polyphenols as active principles but also incorporates stabilizing agents, preservative agents, and antioxidant agents to enhance the stability, efficacy, and shelf-life of the formulation. These additional components ensure that the active ingredients remain effective over time, are properly absorbed, and are protected from degradation, microbial contamination, and oxidation.

The stabilizing agents are essential for maintaining the solubility and homogeneity of the composition, particularly for cannabinoids, which are lipophilic and require assistance to remain soluble in aqueous formulations. In a further embodiment, the stabilizing agents range from 12% to 16%, offering a balance that ensures the cannabinoids, flavonoids, and polyphenols are delivered efficiently and remain bioavailable. In a preferred embodiment, the stabilizing agents include Propylene Glycol 400, Sorbitan 80, and Polysorbate 80. Propylene Glycol 400 acts as a solubilizing agent, ensuring that cannabinoids such as CBD and CBG remain in solution. Sorbitan 80 and Polysorbate 80 are emulsifying agents that help maintain the stability of the formulation, preventing separation and ensuring uniform distribution of active ingredients throughout the product.

In this preferred embodiment, Propylene Glycol 400 is present at 2.67%, Sorbitan 80 at 6.67%, and Polysorbate 80 at 6.67%. This specific combination and concentration of stabilizing agents work synergistically to ensure that the active principles are properly solubilized and remain stable, providing consistent therapeutic efficacy throughout the duration of the treatment.

Preservative agents are included to protect the formulation from microbial contamination, which is critical for maintaining the safety and efficacy of the product over time. In further embodiments, the preservative agents range from 0.1% to 2.0%, ensuring that the composition is protected against microbial growth without compromising the effectiveness of the active ingredients. In a preferred embodiment, the preservative agents include Methylparaben and Propylparaben, both of which are widely used and effective at preventing bacterial and fungal contamination. In a further preferred embodiment, Methylparaben is present at 0.18% and Propylparaben at 0.02%, providing a balanced approach to preserving the composition without overwhelming the formulation with preservatives.

Antioxidant agents are another critical component of the formulation, as they protect the cannabinoids, flavonoids, and polyphenols from oxidative degradation. Oxidation can lead to a loss of potency and efficacy in these active ingredients, so the inclusion of antioxidants ensures that the formulation remains effective over time. In further embodiments, the antioxidants range from 0.07% to 0.10%, which is sufficient to prevent oxidative damage without interfering with the bioavailability of the active ingredients. In a preferred embodiment, the antioxidant agent used is vitamin E, which is known for its strong antioxidant properties. In this embodiment, vitamin E is present at 0.1%, offering robust protection against oxidation while also providing additional skin benefits, such as promoting skin healing and reducing inflammation, which is especially beneficial in treating shingles lesions.

In another embodiment, the composition may include a different combination of stabilizing agents, preservatives, and antioxidants tailored to specific formulation needs. For instance, the composition could include Propylene Glycol 400 at 3%, Sorbitan 80 at 7%, and Polysorbate 80 at 6%. This variation allows for a more fluid and easily absorbable formulation, ideal for patients who require faster absorption of the active ingredients.

In a different formulation, the preservatives may include Benzoic Acid and Potassium Sorbate. For example, the composition could include Benzoic Acid at 0.15% and Potassium Sorbate at 0.05%, providing a more natural preservative system for patients with sensitivities to parabens. This alternative would offer the same protection against microbial growth while catering to patients who prefer preservative systems derived from more natural sources.

Another possible formulation could adjust the antioxidant system. Instead of solely relying on vitamin E, the composition could include a combination of vitamin E and Vitamin C at 0.05%, enhancing the antioxidant protection. Vitamin C adds an extra layer of defense against oxidative stress and can support the overall immune response, making it particularly useful for patients dealing with recurrent viral infections or those in need of stronger immune support.

In one embodiment designed for long-term stability and higher preservation needs, the concentration of preservatives could be adjusted, with Methylparaben at 0.2% and Propylparaben at 0.03%. This increase in preservative concentration could be beneficial for products stored in humid environments or for patients requiring longer shelf-life stability, ensuring the formulation remains free of microbial growth over an extended period.

For formulations designed for more sensitive skin or patients with allergies, the antioxidant system could include a lower concentration of vitamin E at 0.07%, combined with EDTA at 0.03%, offering a gentler antioxidant effect that still provides sufficient protection from oxidation. This combination would be ideal for patients with sensitive skin or those prone to allergic reactions, as it minimizes the potential for skin irritation while maintaining the efficacy of the formulation.

These stabilizing, solubilizing, preservative, and antioxidant systems work in tandem with the active ingredients—cannabinoids, flavonoids, and polyphenols—by ensuring their bioavailability, efficacy, and stability throughout the product's shelf life. For example, a composition including 8.33% CBD, 3.33% CBG, 16.67% quercetin, and 8.33% resveratrol would maintain its therapeutic effectiveness over time, thanks to the presence of these carefully selected stabilizers and antioxidants. The solubilizing agents would ensure the cannabinoids are properly absorbed by the body, while the preservatives and antioxidants would protect the composition from degradation, ensuring consistent relief from inflammation, pain, and viral activity associated with shingles.

By offering these various combinations of stabilizing agents, preservatives, and antioxidants, the formulation can be tailored to meet specific patient needs, offering flexible solutions for both acute treatment and long-term symptom management. This approach guarantees that the therapeutic potential of cannabinoids, flavonoids, and polyphenols is fully realized, delivering an effective and stable treatment option.

In another embodiment, the composition additionally contains stabilizing agents, preservative agents and antioxidant agents. In a further embodiment, the stabilizing agents range from 12% to 16%, preservative agents range from 0.1% to 2.0% and the antioxidants from 0.07% to 0.10%. In a preferred embodiment, the composition comprises stabilizing agents such as Propylene Glycol 400, Sorbitan 80 and Polysorbate 80, preservative agents such as Methylparaben, and Propylparaben, and antioxidants such as vitamin E. In a further preferred embodiment, the composition comprises Propylene Glycol 400 at 2.67%, Sorbitan 80 6.67%, Polysorbate 80 6.67%, Methylparaben 0.18%, Propylparaben 0.02%, and vitamin E at 0.1%.

In some embodiments, this pharmaceutical composition is designed for multiple routes of administration and pharmaceutical forms. In a further embodiment, the routes of administration may include oral, sublingual, topic, inhalation, parenteral, ophthalmic, nasal, otic and intravaginal, while the pharmaceutical forms include tablets, capsules, oral drops, syrups, suspensions, powders for oral reconstitution, elixirs, creams, emulsions, oral solutions, gummies, caramels, sprays, oral topicals, mouth rinses, sublingual tablets, sublingual films, ovules, suppositories, injectable solutions, and injectable suspensions. In a preferred embodiment, this pharmaceutical composition is intended for oral administration and presented in capsules.

One embodiment involves a phytocannabinoid-based composition formulated into oral dosage forms, such as tablets, capsules, oral drops, and intravaginal ovules, or intended for ingestion and gastrointestinal absorption. These forms can include immediate-release, sustained-release, or controlled-release options to regulate the bioavailability of cannabinoids over a period. In some embodiments, these forms may include immediate-release, sustained-release, or controlled-release options to regulate cannabinoid bioavailability over time.

Product 2—topical cream: is a composition comprising cannabinoids and alkaloids, specifically: The topical treatment comprises a topical cream. The topical cream comprises: a) an active principle comprising at least two cannabinoids; b) an active principle comprising at least one alkaloid and aloe vera and, c) an emollient agent.

The topical treatment comprises a carefully formulated topical cream designed to deliver targeted relief from the localized symptoms of shingles, such as pain, itching, and inflammation. This cream offers a multi-pronged therapeutic approach by combining active cannabinoids, alkaloids, aloe vera, and emollient agents to address the discomfort associated with herpetic lesions while promoting faster healing and skin recovery.

The topical cream contains a) an active principle comprising at least two cannabinoids, such as cannabidiol (CBD) and cannabigerol (CBG), both of which are known for their powerful anti-inflammatory, analgesic, and neuroprotective properties. CBD acts as a potent anti-inflammatory agent that helps reduce redness, swelling, and irritation caused by shingles, while also providing relief from nerve pain and discomfort. CBG adds further therapeutic benefit by supporting nerve protection and repair, making it particularly effective in addressing neuralgic pain and reducing the risk of postherpetic neuralgia. Together, these cannabinoids interact with the skin's cannabinoid receptors, modulating pain and inflammation pathways to provide fast and effective relief from the symptoms of shingles.

In addition to cannabinoids, the cream also includes b) an active principle comprising at least one alkaloid and aloe vera. The alkaloid in the formulation, such as capsaicin, is a crucial ingredient for managing pain, particularly the burning or tingling sensations associated with shingles. Capsaicin works by desensitizing pain receptors in the skin, helping to reduce the sensation of pain over time. This provides immediate relief from the discomfort caused by shingles lesions, making daily life more manageable for patients. Aloe vera, known for its soothing, hydrating, and anti-inflammatory properties, complements the action of capsaicin and cannabinoids. Aloe vera not only offers a cooling effect that helps alleviate irritation and itching but also promotes skin healing by moisturizing and repairing damaged tissues. Its antioxidant properties further protect the skin from oxidative stress, which can delay healing and exacerbate inflammation.

To ensure that the cream is comfortable to apply and enhances skin recovery, the formulation also includes c) an emollient agent. The emollient is designed to keep the skin hydrated, soft, and supple, preventing dryness and cracking that often occur around shingles lesions. Emollient agents such as glycerin, shea butter, or medium chain triglycerides (MCT oil) may be included to provide deep moisturization and create a protective barrier on the skin, locking in moisture and promoting healing. These agents also help enhance the absorption of the active ingredients, ensuring that the cannabinoids, alkaloids, and aloe vera are delivered effectively to the affected areas. In one preferred embodiment, MCT oil serves as the emollient agent, known for its ability to improve the bioavailability of lipophilic compounds like cannabinoids, ensuring maximum therapeutic effect.

The combination of these ingredients offers a comprehensive solution to the localized symptoms of shingles. The cannabinoids provide potent anti-inflammatory and pain-relieving effects, while the alkaloid capsaicin offers targeted pain relief by desensitizing the skin's pain receptors. Aloe vera acts as a soothing and healing agent, promoting skin repair and reducing inflammation. The emollient agents ensure that the skin remains hydrated and protected, which is crucial for the overall recovery process.

In another embodiment, the topical cream may include CBD at a concentration of 3%, CBG at 1%, capsaicin at 0.1%, aloe vera at 10%, and an emollient agent such as MCT oil at 5%. This formulation is designed to provide balanced pain relief, inflammation reduction, and skin hydration. The moderate concentration of cannabinoids ensures effective relief from pain and inflammation, while the inclusion of capsaicin provides additional desensitization of pain receptors for those experiencing severe shingles-related pain. Aloe vera and the emollient agent work together to promote skin healing, ensuring that the cream not only alleviates symptoms but also supports the long-term recovery of damaged skin.

For patients with more severe symptoms, a higher concentration of active cannabinoids may be included in the cream. For instance, the cream may comprise CBD at 5%, CBG at 2%, capsaicin at 0.2%, aloe vera at 15%, and an emollient agent like shea butter at 7%. This formulation is ideal for patients suffering from intense pain, inflammation, and irritation, providing a stronger anti-inflammatory and analgesic effect while ensuring that the skin remains moisturized and protected from further damage.

In another embodiment, for those who require a lighter formula for mild symptoms or for maintenance after the acute phase of shingles, the topical cream may contain CBD at 2%, CBG at 0.5%, capsaicin at 0.05%, aloe vera at 8%, and MCT oil at 3%. This formulation focuses on maintaining skin health and providing ongoing relief from mild irritation or discomfort, while still promoting healing and protection from long-term complications.

By integrating cannabinoids, alkaloids, aloe vera, and emollient agents, the topical cream offers a comprehensive and highly effective treatment for the localized symptoms of shingles. This combination not only addresses the immediate needs of pain relief and inflammation reduction but also supports the skin's natural healing process, ensuring that the affected areas recover fully and without lasting damage.

In some embodiments, the method for treating shingles in a human individual comprises administering an effective concentration of the composition, wherein the active principle comprises cannabinoids, flavonoids, polyphenols, alkaloids, and stabilizing solubilizing agents.

In one embodiment, the topical cream formulation for shingles treatment is designed to incorporate a potent combination of cannabinoids, alkaloids, and aloe vera, targeting pain relief, inflammation reduction, and skin healing. The active principles in this composition include Cannabidiol (CBD) and Cannabigerol (CBG) as the primary cannabinoids, along with an alkaloid, capsaicin, known for its powerful pain-relieving properties. The cream is further enhanced by the inclusion of aloe vera, which provides soothing, anti-inflammatory, and hydrating benefits.

In a preferred embodiment, the composition includes cannabinoids such as CBD and CBG at concentrations ranging from 3% to 4%, providing anti-inflammatory and analgesic effects that help manage the localized pain and irritation caused by shingles. CBD at a concentration of 2.27% and CBG at 1.51% work together to target both nerve pain and skin inflammation. CBD reduces inflammation and promotes skin healing, while CBG provides neuroprotective effects, reducing the likelihood of long-term nerve damage and complications like postherpetic neuralgia.

The alkaloid component of the formulation is capsaicin, which is included at a concentration ranging from 0.2% to 0.3%. Capsaicin acts by desensitizing pain receptors in the skin, offering immediate relief from the burning and tingling sensations that shingles patients often experience. In a further preferred embodiment, the cream contains capsaicin at a concentration of 0.23%, which strikes an optimal balance between providing effective pain relief and minimizing skin irritation. Capsaicin helps disrupt the pain signal pathways in the nerves, which makes it particularly effective for addressing the acute nerve pain associated with shingles lesions.

In another embodiment, the composition contains a significant proportion of aloe vera gel, ranging from 28% to 32%, to promote skin hydration, reduce inflammation, and support faster healing of shingles lesions. Aloe vera is well-known for its soothing and moisturizing properties, helping to calm irritated skin and reduce itching, while also accelerating tissue repair. In a preferred embodiment, the composition includes 30% aloe vera gel, which provides a substantial hydrating base, ensuring that the skin remains soft and protected as the cannabinoids and capsaicin work to reduce inflammation and pain.

A specific embodiment of the cream formulation could include the following concentrations per 100 g of product: 30 g of aloe vera gel, 2.27 g of CBD (2.27%), 1.51 g of CBG (1.51%), and 0.23 g of capsaicin (0.23%). This formulation is applied four times a day for four weeks to provide continuous relief from shingles symptoms, ensuring that the skin remains hydrated, inflammation is reduced, and pain is consistently managed. In one embodiment, the topical cream may include higher concentrations of cannabinoids for patients experiencing more severe pain and inflammation. The formulation could include CBD at 3.5%, CBG at 1.75%, capsaicin at 0.3%, and aloe vera gel at 28%. This combination delivers stronger anti-inflammatory and analgesic effects while maintaining a high level of skin hydration. This formulation is ideal for individuals dealing with intense nerve pain or extensive shingles outbreaks, as the higher concentration of cannabinoids provides enhanced pain relief and anti-inflammatory action.

In another embodiment, the formulation may be tailored for maintenance after the acute phase of shingles, with CBD at 2%, CBG at 1%, capsaicin at 0.2%, and aloe vera gel at 32%. This lighter formulation focuses on maintaining skin health and providing ongoing relief from any lingering discomfort or mild irritation. The lower concentration of cannabinoids is sufficient to manage mild symptoms, while the higher concentration of aloe vera ensures continued hydration and protection of the skin as it heals. In a balanced embodiment for patients experiencing moderate symptoms, the composition may include 2.5% CBD, 1.5% CBG, 0.25% capsaicin, and 30% aloe vera. This formulation offers a middle-ground solution, providing sufficient pain relief and inflammation control for most patients, while also supporting skin hydration and recovery. The moderate concentration of capsaicin provides adequate desensitization of pain receptors, making it effective for managing ongoing pain without overwhelming sensitive skin.

For patients who require long-term management of chronic pain, such as postherpetic neuralgia, the composition could include 3% CBD, 2% CBG, 0.23% capsaicin, and 28% aloe vera. This formulation is designed to offer sustained relief from nerve pain while ensuring that the skin remains moisturized and protected. The combination of a higher concentration of CBG with capsaicin helps manage nerve pain more effectively, while the CBD and aloe vera provide continuous support for skin health and inflammation reduction. In another embodiment, for patients with sensitive skin or those who may react to higher concentrations of active ingredients, the formulation may include 1.5% CBD, 1% CBG, 0.2% capsaicin, and 32% aloe vera gel. This high concentration of aloe vera ensures that the skin is well-hydrated and soothed, while the cannabinoids and capsaicin still provide pain relief and anti-inflammatory benefits. This formulation is ideal for patients with sensitive skin who need a gentler approach to managing their shingles symptoms.

By incorporating cannabinoids, capsaicin, aloe vera, and emollient agents, the topical cream can be customized to meet a variety of patient needs, from intense symptom management during the acute phase of shingles to long-term maintenance of skin health and pain relief. Each formulation provides a comprehensive approach to treating shingles, combining pain relief, inflammation reduction, and skin healing in one versatile product. These multiple combinations ensure that the cream can be tailored to the specific severity of symptoms, patient preferences, and treatment goals, offering a flexible and effective solution for shingles care.

In one further embodiment, the active principle comprises (Cannabidiol) CBD and Cannabigerol (CBG). In a further embodiment, the composition comprises one alkaloid. In a preferred embodiment the alkaloid is capsaicin. In a further preferred embodiment, the cream includes capsaicin in addition to the cannabinoids. In a further embodiment, the composition comprises aloe vera. In a further embodiment, the composition includes alkaloids as capsaicin ranging from 0.2% to 0.3%. In one further embodiment, the composition includes alkaloids as capsaicin at a concentration of 0.23% for topical cream. In a further embodiment, the composition includes aloe vera ranging from 28% to 32%. In one further embodiment, the composition includes alkaloids as capsaicin at a concentration of 30% for topical cream. In further embodiments, the composition is administered in the form of 1 application, each containing a concentration of: Aloe Vera Gel: 30 g, CBD: 2.27 g, CBG: 1.51 g, Capsaicin: 0.23 g per 100 g, administered 4 times a day for 4 weeks. In some embodiments, the composition includes cannabinoids and alkaloids as active principles. In a further embodiment, the composition comprises cannabinoids ranging from 3% to 4% and alkaloids ranging from 0.2% to 0.3%. In a preferred embodiment, the composition comprises CBD and CBG. In a preferred embodiment, the composition comprises Capsaicin. In a further preferred embodiment, the composition comprises CBD 2.27%, CBG 1.51%. In a further preferred embodiment, the composition comprises Capsaicin 0.23%.

In one embodiment, the pharmaceutical composition is enhanced by the inclusion of various excipients or carriers, which play a critical role in improving the stability, bioavailability, and overall patient acceptability of the formulation. These excipients are specifically selected to support the efficacy and shelf life of the active ingredients, such as cannabinoids, flavonoids, and alkaloids, ensuring that they remain stable and potent throughout the product's lifecycle. By facilitating the absorption and distribution of cannabinoids like CBD and CBG, the excipients help maximize the therapeutic effects, ensuring that the active compounds are delivered efficiently to the areas where they are most needed.

The inclusion of emulsifiers, preservative agents, antioxidant agents, solvents, viscosity agents, and emollient agents provides additional support to the formulation, ensuring its integrity and effectiveness over time. These components typically range from 65% to 67% of the overall composition, providing a stable base that enhances the solubility, consistency, and application of the cream. This balance ensures that the formulation remains homogenous and easy to apply, while also extending its shelf life and preventing the degradation of the active ingredients.

Emulsifiers are crucial for ensuring that the lipophilic cannabinoids are evenly dispersed within the cream. Common emulsifying agents, such as Sorbitan, Polysorbate 80, and Lecithin, help maintain the stability of the cream by preventing the separation of oil and water components. These emulsifiers create a smooth, even texture, allowing for a consistent application and absorption of the active ingredients across the skin.

Preservative agents, such as Methylparaben and Propylparaben, are included to protect the formulation from microbial contamination and to ensure the cream remains safe for use over its intended shelf life. In this embodiment, the preservative agents typically range from 0.1% to 2.0%, providing sufficient protection against bacterial or fungal growth without compromising the effectiveness or safety of the product. These preservatives help maintain the integrity of the formulation, especially in products that are used over extended periods.

Antioxidant agents, such as Vitamin E, are also added to the composition to prevent the oxidation of sensitive compounds like cannabinoids, which can degrade when exposed to air, light, or heat. In this embodiment, the antioxidant agents typically range from 0.07% to 0.10%. Vitamin E not only prevents oxidative degradation but also offers additional skin benefits, including moisturizing and soothing effects, which enhance the overall therapeutic efficacy of the cream.

Solvents such as Propylene Glycol and Ethanol may be incorporated into the formulation to improve the solubility and absorption of the active ingredients. These solvents help to dissolve cannabinoids and other bioactive compounds, ensuring that they are properly absorbed into the skin and delivered to the affected tissues. This ensures that the therapeutic effects are felt quickly and efficiently, making the cream more effective in managing the symptoms of shingles.

Viscosity agents, such as Carbomer or Hydroxyethylcellulose, are used to give the cream the right texture and consistency, ensuring that it spreads easily on the skin and stays in place without running or dripping. These agents help control the thickness of the cream, making it easy to apply and ensuring that the active ingredients are evenly distributed across the skin for maximum effectiveness.

Emollient agents such as Medium Chain Triglycerides (MCT oil), Shea Butter, or Glycerin are also included in the composition to keep the skin hydrated and protected. These agents help to create a moisturizing barrier on the skin, preventing dryness and irritation, which are common symptoms associated with shingles. The emollients also enhance the absorption of cannabinoids and other active ingredients, ensuring that the skin remains soft and hydrated while also receiving the therapeutic benefits of the formulation. In this embodiment, the emollient agents are present in a range that allows for optimal skin hydration while supporting the delivery of the active principles.

In one embodiment, the formulation may include Vitamin E as the primary antioxidant at 0.10%, combined with a higher concentration of emollients such as MCT oil at 10% and Shea Butter at 5%. This combination ensures that the cream not only provides antioxidant protection to the cannabinoids but also offers enhanced moisturizing properties, making it ideal for patients with sensitive or dry skin. The addition of these emollients helps to create a protective layer on the skin, which is especially beneficial for shingles patients who experience dryness and irritation around the lesions.

In another embodiment, the composition may include Propylene Glycol at 7% and Ethanol at 5%, providing a robust solvent system that enhances the solubility and absorption of cannabinoids like CBD and CBG. This formulation is particularly suitable for patients who require faster relief from pain and inflammation, as the solvents help to increase the bioavailability of the active ingredients, ensuring that they are absorbed quickly into the skin.

In a balanced formulation for optimal texture and application, the composition could include Polysorbate 80 at 6%, Sorbitan at 4%, and Carbomer at 1%. This combination of emulsifying and viscosity agents ensures that the cream has a smooth, stable consistency, making it easy to apply while maintaining the structural integrity of the product. The emulsifiers help to keep the cannabinoids evenly dispersed, while the viscosity agent ensures that the cream stays in place on the skin, providing consistent therapeutic effects.

For a formulation that emphasizes long-term stability and preservation, the composition may include Methylparaben at 0.15%, Propylparaben at 0.05%, and Vitamin E at 0.08%. This combination of preservative and antioxidant agents ensures that the cream remains free from microbial contamination and oxidation for an extended period, making it ideal for patients who need a stable product for long-term use.

By incorporating these excipients and carriers, along with active ingredients such as cannabinoids, alkaloids, and aloe vera, the formulation is designed to be both effective and patient-friendly. The excipients not only enhance the stability and bioavailability of the active ingredients but also improve the overall texture, absorption, and safety of the cream. This comprehensive approach ensures that the cream provides lasting relief from the symptoms of shingles while remaining gentle and moisturizing for the skin, making it suitable for daily use over the course of treatment.

In another embodiment, the pharmaceutical composition designed for topical application contains a carefully balanced combination of excipients that includes emulsifying agents, preservative agents, antioxidant agents, solvent agents, viscosity agents, and emollient agents. These components are specifically selected to improve the formulation's stability, enhance the bioavailability of active ingredients like cannabinoids, and optimize the overall sensory experience for the patient. By ensuring the proper distribution, absorption, and protection of the active compounds, these agents contribute to the composition's therapeutic efficacy and shelf life.

The emulsifying agents, which range from 3.0% to 5.0% in concentration, are responsible for maintaining the homogeneity of the cream by blending water and oil-based components, ensuring a smooth and stable texture. Cetyl Alcohol is a preferred emulsifier in this formulation, contributing not only to emulsification but also to skin conditioning and hydration. At a concentration of 3%, Cetyl Alcohol provides the cream with a stable consistency while enhancing its ability to keep the cannabinoids (CBD and CBG) well-dispersed throughout the product. This ensures that the cannabinoids are uniformly distributed and effectively absorbed by the skin.

The preservative agents protect the cream from microbial contamination, ensuring that it remains safe and effective for the duration of its shelf life. In this embodiment, the preservative agents range from 0.1% to 0.2%, with Potassium Sorbate being the preferred choice due to its efficacy in preventing fungal and bacterial growth. At a concentration of 0.1%, Potassium Sorbate provides adequate preservation without affecting the cream's texture or therapeutic properties. This helps maintain the product's integrity during storage, particularly in environments where temperature and humidity fluctuations are common.

The solvent agents, which range from 3.0% to 6.0%, are crucial for dissolving and facilitating the absorption of lipophilic ingredients like cannabinoids. In a preferred embodiment, Alcohol is used as the primary solvent, with a concentration of 6%. Alcohol helps to dissolve the cannabinoids and other active ingredients, ensuring that they are efficiently absorbed through the skin. It also acts as a penetration enhancer, allowing for faster onset of the therapeutic effects by facilitating the delivery of CBD, CBG, and capsaicin into the deeper layers of the skin.

The viscosity agents in the composition, which range from 0.5% to 1.0%, provide the cream with the desired thickness and texture, ensuring ease of application and uniform coverage. Hydroxyethyl Cellulose, a preferred viscosity agent, is included at 0.5% to maintain a smooth, gel-like consistency. This agent not only provides stability to the formulation but also enhances the sensory experience by ensuring that the cream spreads evenly across the skin without leaving a greasy residue.

The emollient agents in the formulation are responsible for keeping the skin hydrated, soft, and protected, which is particularly important for individuals with shingles, as the condition often results in dry, irritated, and inflamed skin. Emollients range from 5.0% to 10% in concentration. In this embodiment, the combination of Glycerin at 3.0% and Dimethicone at 2.0% provides excellent moisturizing and protective benefits. Glycerin is a humectant that draws moisture into the skin, helping to prevent dryness and promote healing. Dimethicone, a silicone-based emollient, forms a protective barrier on the skin, locking in moisture and protecting sensitive areas from further irritation or damage. This combination helps maintain the skin's moisture balance while also allowing the active cannabinoids and capsaicin to work effectively without causing additional irritation.

The antioxidant agents, present at 0.07% to 0.1%, protect the active ingredients from oxidative degradation. In a preferred embodiment, Vitamin E is used at a concentration of 0.1%, providing robust antioxidant protection that ensures the stability and potency of the cannabinoids, polyphenols, and flavonoids throughout the product's lifespan. Vitamin E also offers additional skin benefits, such as reducing inflammation and supporting skin repair, making it an ideal addition to a cream intended for shingles treatment.

In one embodiment, the emollient concentration may be increased to provide extra hydration and skin protection. The formulation could include Glycerin at 5% and Dimethicone at 3%, offering enhanced moisturizing benefits for individuals with particularly dry or sensitive skin. This higher concentration of emollients ensures that the skin remains hydrated, even in the presence of drying agents like Alcohol. Cetyl Alcohol could remain at 3%, and Potassium Sorbate could be maintained at 0.1%, ensuring stability without compromising on skin protection.

In another embodiment, the focus may be on faster absorption and deeper penetration of the active ingredients. The formulation could include Alcohol at 5% and Propylene Glycol at 3%, providing a dual solvent system that enhances the solubility and penetration of cannabinoids and capsaicin. This combination would allow for rapid absorption of the active principles, offering faster relief from pain and inflammation. Hydroxyethyl Cellulose could remain at 0.5% to maintain the cream's stability and spreadability, while Vitamin E at 0.1% would protect the formulation from oxidative degradation.

In an embodiment designed for long-term use, the composition could balance emollients and viscosity agents to ensure sustained hydration and ease of use. The formulation may include Glycerin at 4%, Dimethicone at 2%, and Hydroxyethyl Cellulose at 0.75%, providing a creamy texture that spreads easily while keeping the skin moisturized for extended periods. Cetyl Alcohol at 4% would enhance the cream's stability, while Potassium Sorbate at 0.15% ensures adequate preservation over time.

For individuals with sensitive skin or for those who require additional antioxidant protection, the formulation may include Vitamin E at 0.1% combined with a secondary antioxidant such as Vitamin C at 0.05%. This combination offers enhanced protection against oxidative stress, which can be particularly beneficial for patients with chronic shingles or recurring outbreaks. The emollient concentration could be maintained at 5%, ensuring that the skin remains soft and protected.

By incorporating these emulsifiers, solvents, viscosity agents, emollients, and antioxidants, the composition is optimized for therapeutic efficacy, stability, and patient comfort. The careful balance of these components ensures that the cannabinoids, alkaloids, and other active ingredients are delivered effectively to the skin, offering relief from pain, inflammation, and irritation associated with shingles. This comprehensive approach ensures that the cream is both effective in treating the symptoms of shingles and gentle on the skin, making it suitable for daily use over the course of the treatment.

In one embodiment, the pharmaceutical composition may further include various excipients or carriers to enhance stability, bioavailability, and patient acceptability, supporting both the efficacy and shelf life of the formulation. These excipients are chosen based on their ability to facilitate the absorption and distribution of the active cannabinoids while maintaining the overall integrity of the composition. In another embodiment, the composition additionally contains emulsifiers agents, preservative agents and antioxidant agents, solvents agents, viscosity agents, and emollient agents. In another embodiment, the range of these components is range from 65% to 67%

It should be noted that the properties described in various embodiments herein could be combined in different ways to create additional embodiments of the present disclosure. Further aspects and features of the disclosure will be evident to those skilled in the art.

In another embodiment, the composition additionally contains emulsifiers agents, preservative agents and antioxidant agents, solvents agents, viscosity agents, and emollient agents. In a further embodiment, the emulsifiers agents range from 3.0% to 5.0%, preservative agents range from 0.1% to 0.2% solvents range from 3.0% to 6.0%, viscosity agents ranging from 0.5% to 1.0%, emollients range from 5.0% to 10%, and the antioxidants from 0.07% to 0.1%. In a preferred embodiment, the composition comprises emulsifiers agents such as Cetyl Alcohol, preservative agents such as Potassium Sorbate, solvents agents such as Alcohol, viscosity agents such as Hydroxyethyl Cellulose, emollients agents such as Glycerin and Dimethicone, and antioxidants such as vitamin E. In a further preferred embodiment, the composition comprises Cetyl Alcohol at 3%, Potassium Sorbate at 0.1%, Alcohol at 6%, Glycerin at 3.0%; Dimethicone at 2.0%, and vitamin E at 0.1%.

In some embodiments, this pharmaceutical composition is designed for multiple routes of administration and pharmaceutical forms. In a further embodiment, the routes of administration may include oral, sublingual, topic, inhalation, parenteral, ophthalmic, nasal, otic and intravaginal, while the pharmaceutical forms include tablets, capsules, oral drops, syrups, suspensions, powders for oral reconstitution, elixirs, creams, emulsions, oral solutions, gummies, caramels, sprays, oral topicals, mouth rinses, sublingual tablets, sublingual films, ovules, suppositories, injectable solutions, and injectable suspensions. In a preferred embodiment, this pharmaceutical composition is intended for topical administration and presented in topical cream.

### Method of Treatment

Aspects of the disclosure provide a method to treat symptoms associated with shingles using compositions comprising phytocannabinoids: (CBD, CBG), flavonoids and polyphenols specifically aimed at patients who are going through shingles as a result of a viral disease caused by the reactivation of the varicella-zoster virus (VZV), which is the same virus that causes chickenpox. After an initial chickenpox infection, the virus remains inactive in the nerve ganglia and can reactivate years later, leading to shingles.

The methods provided are designed to treat individuals, such as humans, by administering orally an effective amount of a composition based on nanoemulsions containing cannabinoids and natural compounds for treating shingles disease. The methods provided are designed to treat individuals, such as humans, by administering a topic cream in an effective amount of a composition based on emulsions containing cannabinoids and natural compounds for treating shingles disease.

In further embodiments, this composition exhibits potential antiviral properties that may aid in the treatment of shingles by affecting the viral entry, replication, or assembly processes. Cannabinoids are suggested to possess broad-spectrum antiviral activity. In this embodiment, the formulation aims to alleviate symptoms caused by the varicella-zoster virus by inhibiting viral replication by targeting DNA polymerase, glycoproteins, the viral envelope, and viral proteins.

In some embodiments, the composition comprises active phytocannabinoid compounds, including CBD and CBG, along with flavonoids, polyphenols, and alkaloids, which may work synergistically to support immune response and overall health during shingles outbreaks. Additionally, in this embodiment, the pharmaceutical composition prevents disease spread, reduces inflammation, and blocks cytokines involved in immune responses. In some embodiments, the cannabinoid pharmaceutical composition may also include other therapeutic agents such as analgesics, anti-inflammatories, and antipruritics, which reduce neuropathic pain and are anti rubefacient to enhance the efficacy of the cannabinoids or provide complementary therapeutic benefits.

In various embodiments, supportive measures to boost immune function and mitigate symptoms are crucial in optimizing outcomes for individuals with compromised immune systems affected by shingles. In some embodiments, a capsule and ointment specifically tailored to address the distinct phases of shingles disease. Shingles progresses through several stages: The pre-eruptive phase is characterized by abnormal skin sensations or pain within the affected dermatome, often accompanied by headaches, malaise, and light sensitivity.

In one embodiment, the acute eruptive phase for shingles is marked by the appearance of painful vesicles or swellings along nerve pathways, typically on one side of the body or face, progressing from macules to fluid-filled vesicles that may ulcerate and crust over, posing high contagion risk until lesions dry.

In other embodiments, the chronic phase follows, featuring persistent pain lasting beyond four weeks post-eruption, accompanied by symptoms like tingling, hypersensitivity, and ongoing discomfort. In this embodiment, the capsule addresses systemic symptoms and supports immune modulation throughout all stages, while the ointment provides targeted relief from pain, itching, and inflammation associated with shingles lesions, ensuring comprehensive management tailored to varying patient needs.

Oral therapy involves delivering the active ingredient of a drug through the mouth, where it is absorbed in the stomach or intestines and enters the bloodstream to exert its therapeutic effects. Oral cannabidiol (CBD) is particularly convenient and recommended for patients suffering from infectious diseases like herpes zoster. This method allows patients to easily ingest medication, making it straightforward to administer and widely accepted among medical professionals and patients alike.

In some embodiments, the cannabinoids used are CBD and CBG. Both are non-intoxicating compounds derived from the cannabis plant known for its potential therapeutic benefits. It interacts with the body endocannabinoid system, which plays a role in regulating various physiological processes such as pain sensation, inflammation, immune response, and mood. CBD is widely studied for its anti-inflammatory properties and its ability to alleviate pain and discomfort associated with conditions like shingles.

In some embodiments, the CBD used in In this embodiments refers to cannabidiol derived from the cannabis plant, which includes a wide range of cannabinoids and beneficial compounds, excluding tetrahydrocannabinol (THC). CBD retains the synergistic effects of multiple cannabinoids, terpenes, and other phytochemicals found in the plant, contributing to its therapeutic potential. It is known for its anti-inflammatory, analgesic, and antioxidant properties, which can help alleviate pain, reduce inflammation, and support immune function in conditions like shingles.

In various embodiments the CBD has shown promise in inhibiting viral replication and modulating immune responses, making it a valuable component in formulations targeting shingles disease. Its versatility and favorable safety profile contribute to its growing popularity in therapeutic applications. In some embodiments, CBG has shown its anti-inflammatory and analgesic properties, facilitating the improvement of the herpetic process in its different phases.

In this embodiment, flavonoid, particularly quercetin, can therefore offer comprehensive therapeutic benefits by addressing inflammation, oxidative stress, immune response, and nerve health simultaneously. In some embodiments, quercetin, a flavonoid, plays a crucial role in managing shingles due to its potent antioxidant and anti-inflammatory properties. By scavenging free radicals and reducing oxidative stress, quercetin protects cells from damage during varicella-zoster virus (VZV) infection.

In one embodiment, flavonoids, particularly quercetin modulates inflammatory pathways, inhibiting cytokine production and alleviating symptoms such as pain and swelling associated with the shingles rash. Quercetin immune-modulating effects enhance the body defense against VZV, potentially reducing viral replication and supporting faster recovery. In some embodiments, its neuroprotective properties may help mitigate nerve pain and prevent complications like postherpetic neuralgia (PHN).

In some embodiments, polyphenols, particularly, resveratrol, a potent polyphenolic compound in the treatment of shingles has notable antioxidant and anti-inflammatory properties. Resveratrol acts by scavenging free radicals, thereby reducing oxidative stress caused by the varicella-zoster virus (VZV) and protecting cells from damage. Its ability to modulate inflammatory pathways helps to alleviate symptoms such as pain and inflammation associated with the shingles rash.

In more than one embodiment, resveratrol also enhances immune function against VZV, potentially inhibiting viral replication and promoting faster recovery. Additionally, its neuroprotective properties may contribute to easing nerve pain and reducing the risk of complications such as postherpetic neuralgia (PHN). Including resveratrol in treatments like. These embodiments for shingles offer a multifaceted approach, addressing inflammation, oxidative stress, immune response enhancement, and nerve health support comprehensively.

In some embodiments, the topical cream is specifically designed for application three times daily to areas affected by conditions like shingles, providing targeted relief from pain, itchiness, and inflammation. The high concentration of CBD aims to alleviate neuropathic pain and reduce inflammation, while capsaicin offers local anesthesia to relieve discomfort.

In one embodiment, capsaicin had a potential use in pain relief. Capsaicin is found in topical creams and ointments to treat neuropathic pain, and other types of muscle or joint pain and anti-inflammatory Properties. In some embodiments, aloe vera is integrated into treatments for shingles due to its beneficial properties in soothing and healing skin affected by the varicella-zoster virus (VZV). In some embodiments, aloe vera has anti-inflammatory, moisturizing, and wound-healing attributes.

When applied topically, aloe vera gel can provide relief from the itching, burning, and discomfort associated with shingles rash. Its cooling effect helps alleviate skin irritation and inflammation, promoting faster healing of lesions and reducing the risk of secondary infections. In this embodiment, by incorporating aloe vera into the topical cream ensures gentle yet effective relief for shingles patients, enhancing comfort and supporting the overall management of the condition.

In some embodiments, topical antiviral therapy offers distinct advantages over systemic drug administration in the treatment of herpes virus skin infections. These include enhanced convenience, higher drug concentrations at the infection site leading to improved efficacy, and targeted delivery that reduces overall costs and minimizes systemic exposure to potential side effects.

In some embodiments, a significant benefit of topical administration is its favorable adverse effects profile, particularly beneficial when the drug is predominantly localized for local pain management with minimal systemic absorption This approach ensures effective treatment while minimizing systemic impacts, making it a preferred choice in managing conditions like shingles.

In this embodiment, a gel is a semisolid preparation typically applied to the skin or mucous membranes for therapeutic or protective purposes. A gel is often used to deliver medications, treat skin conditions, provide moisture, or protect against irritation. They are characterized by their greasy or oily texture, which helps them adhere to the skin and provide a longer-lasting effect compared to creams or lotions.

In some embodiments, it offers a comprehensive approach for treating shingles by targeting various stages of the varicella-zoster virus (VZV) replication cycle. This includes disrupting viral particles directly through a virucidal effect, inhibiting essential viral glycoproteins to prevent host cell attachment and entry, and blocking the transport of viral capsids into the nucleus.

In some embodiments, the pharmaceutical formulation may be administered topically or systemically to reduce the severity or duration of VZV infections, including shingles, by targeting viral pathways involved in disease progression. In this embodiment, the pharmaceutical formulation aims to disrupt VZV replication effectively, potentially reducing symptom severity and improving overall treatment outcomes for shingles patients.

In this embodiment, the pharmaceutical composition specifically targets glycoproteins of the varicella-zoster virus (VZV) crucial for viral attachment and entry into host cells. These include glycoprotein B (gB), glycoprotein C (gC), and glycoprotein E (gE), which play key roles in mediating viral fusion with host cell membranes and facilitating viral spread. In some embodiments by effectively inhibiting these glycoproteins. In this embodiment, the formulation aims to disrupt the initial stages of viral entry and replication.

In some embodiments, the nanoemulsion ointment and capsule formulation includes bioactive compounds known for their immunomodulatory properties, such as CBD, CBG, Resveratrol, and Quercetin. These ingredients have been shown to regulate immune responses, enhance immune function, and reduce inflammation, thereby supporting the body ability to combat viral infections like shingles.

In some embodiments, treatment is tailored with both an ointment designed for topical application to alleviate symptoms of shingles and a capsule intended for different stages of the disease progression.

In this embodiment, the treatment effectively treats a range of shingles symptoms, which include acute pain characterized by burning, tingling, or shooting sensations along affected nerves. It addresses the formation of painful swellings or vesicles on the skin, often accompanied by itching and sensitivity to touch.

In this embodiment, the pharmaceutical formulation aims to reduce inflammation at the site of the rash, promoting faster healing and minimizing the risk of complications such as bacterial infections. Additionally, it targets the persistent neuralgia or nerve pain that can persist even after the rash has healed, known as postherpetic neuralgia (PHN).

Some embodiments offer flexibility in treatment administration, where the formulation can be taken orally or applied topically depending on the individual needs and the stage of shingles progression. In one embodiment the topical application, targets localized pain relief and soothing effects directly at the site of the rash, especially during the acute eruptive phase.

### Combination and Synergy

In this embodiment, the composition leverages a carefully crafted combination of cannabinoids, flavonoids, polyphenols, and alkaloids to produce a synergistic effect that enhances both antiviral and anti-inflammatory benefits. Cannabidiol (CBD) and cannabigerol (CBG), when combined with bioactive compounds like quercetin and resveratrol, amplify the therapeutic potential of each individual component, offering superior relief from the symptoms associated with shingles compared to their use in isolation. This synergistic interaction ensures that multiple physiological pathways are targeted simultaneously, leading to a more comprehensive treatment of the condition.

The combination of CBD and CBG plays a pivotal role in reducing inflammation and modulating immune responses, particularly through their interaction with CB2 receptors found in the peripheral immune system. By activating CB2 receptors, these cannabinoids help to dampen inflammatory responses, which is crucial in reducing the inflammation that underlies the skin lesions and nerve pain caused by shingles. CBD is well-known for its anti-inflammatory properties, which work to alleviate swelling, redness, and irritation. CBG complements these effects by offering additional neuroprotective benefits, helping to protect nerve cells from damage, which can reduce the severity of pain and mitigate the risk of developing chronic nerve damage, such as postherpetic neuralgia.

Quercetin and resveratrol, as flavonoids and polyphenols, bring powerful anti-inflammatory and antioxidant properties to the formulation. Quercetin helps stabilize the immune response, reducing the overactivation of inflammatory pathways and providing relief from pain and swelling. Resveratrol, known for its antiviral effects, helps inhibit the replication of the varicella-zoster virus (VZV), thereby controlling the spread of the virus and reducing the size and duration of shingles outbreaks. Together, quercetin and resveratrol not only enhance the immune system's ability to fight off viral infections but also prevent oxidative stress, which can further exacerbate skin and nerve damage. Their antioxidant properties ensure that the affected tissues are protected from free radicals, promoting faster recovery and reducing the risk of scarring or lingering skin irritation.

The incorporation of capsaicin in the topical formulation adds another layer of therapeutic benefit, particularly in its ability to provide targeted pain relief. Capsaicin works by desensitizing the TRPV1 receptors (transient receptor potential vanilloid 1) in the skin, which are responsible for transmitting pain signals. By interrupting the pain signaling pathways, capsaicin effectively reduces the burning and tingling sensations that are characteristic of shingles. This localized action makes capsaicin especially effective for addressing the acute, intense nerve pain associated with shingles, offering fast relief without the need for systemic analgesics. In combination with the anti-inflammatory and immune-modulating effects of CBD and CBG, capsaicin provides a more comprehensive approach to managing the severe pain associated with the condition.

The synergistic interaction between these components—CBD, CBG, quercetin, resveratrol, and capsaicin—results in a significant reduction of acute pain, inflammation, and lesion size in shingles patients. By acting on both CB2 receptors and other key molecular targets such as TRPV1 receptors, the composition addresses not only the surface symptoms of shingles, like skin irritation and lesions, but also the deeper inflammatory and immune responses that contribute to nerve pain and viral activity.

For example, in cases of severe shingles outbreaks, the combined action of CBD and CBG modulates the immune response, reducing inflammation around the nerve endings and skin lesions. At the same time, quercetin and resveratrol provide systemic support by curbing viral replication and offering antioxidant protection to the affected tissues. Capsaicin adds an additional layer of targeted relief by specifically addressing the localized pain caused by shingles lesions. This comprehensive approach ensures that multiple aspects of the disease are treated simultaneously, improving patient outcomes and accelerating the healing process.

In one embodiment, the topical formulation may include a higher concentration of CBD (5%) and CBG (2%), along with quercetin (12%) and resveratrol (6%). Capsaicin may be present at 0.3%. This formulation is designed for patients with severe shingles symptoms, providing stronger anti-inflammatory and analgesic effects. The increased cannabinoid concentration ensures that inflammation and pain are quickly brought under control, while quercetin and resveratrol reduce viral activity and support immune function.

In another embodiment, the formulation could include CBD (3%), CBG (1.5%), quercetin (10%), resveratrol (5%), and capsaicin (0.23%). This balanced formula is ideal for patients with moderate shingles symptoms, providing a comprehensive approach to pain management, inflammation reduction, and skin healing. The concentration of cannabinoids offers sufficient pain relief, while quercetin and resveratrol provide support for immune modulation and antiviral activity. Capsaicin delivers effective pain relief for localized areas of intense discomfort.

For patients in the post-acute phase of shingles, a lighter formulation may be more appropriate. This combination could include CBD (2%), CBG (1%), quercetin (8%), resveratrol (4%), and capsaicin (0.2%). This lower-concentration formula focuses on maintaining skin health, preventing further irritation, and providing ongoing relief from residual pain or inflammation. The lower capsaicin concentration ensures gentle pain relief while minimizing any potential skin sensitivity.

In another embodiment, for patients requiring stronger antiviral support, the composition could include higher concentrations of quercetin (15%) and resveratrol (7%), combined with CBD (4%), CBG (1.5%), and capsaicin (0.25%). This formulation would be ideal for patients dealing with recurrent shingles outbreaks or those needing additional immune system support. The elevated levels of flavonoids and polyphenols ensure robust antiviral and antioxidant effects, reducing the risk of prolonged outbreaks.

The combination of cannabinoids and flavonoids in this formulation ensures that multiple therapeutic mechanisms are engaged, from immune modulation and inflammation control to pain relief and antiviral activity. This multi-targeted approach not only improves the immediate management of shingles symptoms but also helps prevent long-term complications such as postherpetic neuralgia, scarring, and chronic skin irritation. By reducing viral load and addressing inflammation at both the systemic and local levels, this composition offers a more effective and comprehensive treatment than any of its individual components could achieve alone.

### Method of Production

In one embodiment, various delivery enhancements are employed to improve the efficacy and targeting of cannabinoid compositions. Methods may include liposomal encapsulation, nanoplatform as nanoemulsions, and targeted delivery systems. In one embodiment, the nanoemulsion technology utilizes advanced nanoemulsion technology to encapsulate CBD, CBG, quercetin, and resveratrol. Nanoemulsions enhance bioavailability, and absorption of active ingredients, ensuring effective systemic delivery.

In a particular embodiment, the selection of reactants, surfactants, and stabilizers is essential during the production process, as it significantly influences the stability, size, and functionality of the nanoplatforms or nanoemulsions. Various surfactants may be incorporated to stabilize the nanoemulsion, with concentration and type adjusted according to the formulation. Options include nonionic, cationic, or anionic surfactants, and the hydrophilic-lipophilic balance (HLB) of the surfactant will impact both the emulsification process and the size of the nanoplatform.

In one embodiment, the nanoplatform compositions include nanoparticles with a mean diameter of less than 200 nm. These particles may be delivered in a sustained-release capsule for optimized therapeutic effects in shingles treatment. In one embodiment, high-energy nano-emulsification methods are used to create stable nanoemulsions for effective delivery.

In different embodiments, the composition may differ in cannabinoid concentrations. Furthermore, the ratios of various cannabinoids can be adjusted to enhance the therapeutic profile while ensuring efficacy. In additional variations, the composition may blend two or more cannabinoids in specific ratios to achieve a synergistic effect, where the overall therapeutic action exceeds that of the individual cannabinoids. In a particular embodiment, the production process may incorporate excipients that modify the surface of the nanoplatforms, allowing for targeted delivery and enhanced interactions with biological systems. Such excipients may encompass stabilizers, antioxidants, preservatives, as well as both oil phase and aqueous vehicles.

In one embodiment, polymers of lipids serve as the primary building blocks for the nanoplatforms. The production method may vary the type and molecular weight of the polymers or the chain length and saturation of the lipids, influencing the mechanical stability and release properties of the nanoplatform.

In one variation, the conditions for producing the nanoplatforms or nanoemulsions are significantly important, affecting the final properties and requiring adjustments for various compositions. Temperature control is essential across different production methods, where maintaining the right temperature is crucial for the formation and stability of the nanoemulsion or nanoplatforms. Increased temperatures may lower the viscosity of the mixture, aiding emulsification, but can also threaten the integrity of sensitive components.

In a particular embodiment, pressure adjustments may be applied during high-pressure techniques, like homogenization, to control the size and distribution of the nanoplatforms. Higher pressures can lead to reduced particle sizes but may also escalate energy expenses and the potential for degrading sensitive compounds. Furthermore, the number of pressure cycles can be fine-tuned to ensure consistent formation of the nanoplatforms.

In a particular embodiment, the method of production may consist of mixing the reactants under high shear or vortex conditions to achieve an even distribution of particles and prevent agglomeration. The speed and duration of mixing can be optimized to obtain the desired particle size and homogeneity, with different mixers, such as rotor-stator mixers, selected based on the composition requirements.

### Production Process Capsules:

In one embodiment, the method of production may involve multiple stages, each designed to refine the properties of the nanoplatforms or nanoemulsions. In one embodiment, the preparation of the oil phase occurs in a suitable container, where one or more oils, such as medium or long-chain triglycerides, essential oils, or bioactive lipids, are combined with lipophilic excipients and any liposoluble active ingredients. In one embodiment, the aqueous phase is prepared simultaneously in another container. This phase consists of purified water along with hydrophilic surfactants and co-emulsifying agents. In one embodiment, preliminary emulsification is carried out after both phases have reached the appropriate temperature, achieved by combining the oil phase and the aqueous phase to form the preliminary emulsion.

In one embodiment, the droplet size of the preliminary emulsion is reduced to a nanometric range through the application of high-energy homogenization techniques, which is crucial for obtaining the desired nanoemulsion. In one embodiment, after homogenization, the resulting nanoemulsion undergoes a controlled cooling process to ensure its stability. In one embodiment, the nanoemulsion is filled into a capsule with rigorous controls during the process.

### Production Process Topical Cream

In one embodiment, the preparation of the oil phase occurs in a suitable container, where one or more oils, such as medium or long-chain triglycerides, or bioactive lipids, are combined with lipophilic excipients and any liposoluble active ingredients.

In one embodiment, the aqueous phase is prepared simultaneously in another container. This phase consists of purified water along with hydrophilic surfactants and co-emulsifying agents. In one embodiment, emulsification is carried out after both phases have reached the appropriate temperature, achieved by combining the oil phase and the aqueous phase to form the emulsion. In one embodiment, the emulsion is filled into tubes or jars with rigorous controls during the process.

### EXAMPLES

### Examples of Pharmaceutical Compositions:

The current example discloses a therapeutic agent aimed at treating shingles. This agent comprises a unique composition that utilizes nanoplatform containing cannabinoids, such as CBD, and CBG. The formulation is defined by designated percentage ranges of each to ensure effective administration. The example describes a substance designed to treat singles in patients. This substance comprises a formulation that utilizes nanoplatform containing cannabinoids such as CBD, and CBG. Each compound percentage range for administration is outlined to optimize therapeutic effects.

Particular amounts of cannabinoids: 50 mg of CBD, 20 mg of CBG actives per capsule. Particular amounts of cannabinoids: CBD: 2.27 g, CBG: 1.51 g per 100 g of the topical cream.
Capsules: Formulation 1:600 mg per capsule

| Component | Quantity (mg) | Percentage (%) |
|---|---|---|
| CBD | 50 | 8.33 |
| CBG | 20 | 3.3 |
| Quercetin | 100 | 16.67 |
| Resveratrol | 50 | 8.33 |
| Medium chain triglycerides | 62.2 | 10.37 |
| Sorbitan 80 (Span 80) | 40 | 6.67 |
| Vitamin E | 0.6 | 0.1 |
| Polysorbate 80 (Tween 80) | 40 | 6.67 |
| Polyethylene glycol 400 | 16 | 2.67 |
| Methylparaben | 1.08 | 0.18 |
| Propylparaben | 0.12 | 0.02 |
| Purified Water | 220 | 36.67 |

Formulation 2:800 mg per capsule

| Component | Quantity (mg) | Percentage (%) |
|---|---|---|
| CBD | 50 | 6.25 |
| CBG | 20 | 2.5 |
| Quercetin | 100 | 12.5 |
| Resveratrol | 50 | 6.25 |
| Medium chain triglycerides | 110 | 13.75 |
| Sorbitan 80 (Span 80) | 40 | 5 |
| Vitamin E | 0.8 | 0.1 |
| Polysorbate 80 (Tween 80) | 40 | 5 |
| Polyethylene glycol 400 | 16 | 2 |
| Methylparaben | 1.44 | 0.18 |
| Propylparaben | 0.16 | 0.02 |
| Purified Water | 371.6 | 46.46 |

### Topical Cream:

Formulation 1:60 g per Collapsible tube.

| Component | Quantity (mg) | Percentage (%) |
|---|---|---|
| Aloe Vera Gel | 18.00 | 30 |
| CBD | 1.36 | 2.27 |
| CBG | 0.91 | 1.51 |
| Capsaicin | 0.14 | 0.23 |
| Medium Chain Triglycerides | 6.00 | 10 |
| Cetyl Alcohol | 1.80 | 3 |
| Dimeticone | 1.20 | 2 |
| Vitamin E | 0.06 | 0.1 |
| Potassium Sorbate | 0.06 | 0.1 |
| Hydroxyethyl Cellulose | 0.30 | 0.5 |
| Glycerin | 1.80 | 3 |
| Ethanol | 3.60 | 6 |
| Purified Water | 24.77 | 41.29 |

Formulation 2:40 g per Collapsible tube.

| Component | Quantity (mg) | Percentage (%) |
|---|---|---|
| Aloe Vera Gel | 12.00 | 30 |
| CBD | 0.91 | 2.27 |
| CBG | 0.60 | 1.51 |
| Capsaicin | 0.09 | 0.23 |
| Medium Chain | 6.00 | 15 |
| Triglycerides | | |
| Cetyl Alcohol | 2.00 | 5 |
| Dimeticone | 2.00 | 5 |
| Vitamin E | 0.04 | 0.1 |
| Potassium Sorbate | 0.08 | 0.2 |
| Hydroxyethyl Cellulose | 0.40 | 1 |
| Glycerin | 2.00 | 5 |
| Ethanol | 1.20 | 3 |
| Purified Water | 12.68 | 31.69 |

### Nanoemulsion Process:

### Example 1

Oil phase: A quantity of 10.37 grams of medium-chain triglycerides (MCT) is added to a suitable container. Subsequently, 6.67 grams of Sorbitan 80 (Span 80), and 0.1 grams of Vitamin E are incorporated. Immediately, 8.33 grams of CBD and 3.33 grams of CBG are added. The mixture is stirred at a speed of 500 revolutions per minute (rpm) until all components are fully integrated. The oil phase is then heated to a temperature between 40° C. and 45° C.

Aqueous phase: Heat 36.67 grams of purified water to 90° C. and dissolve 0.18 grams of methylparaben and 0.02 grams of propylparaben. Cool the solution to a temperature between 40° C. and 45° C. Add 2.67 grams of polyethylene glycol 400 (PEG 400) and 6.67 milligrams of polysorbate 80 (Tween 80), and stir until all ingredients are completely incorporated. Then, add 8.33 grams of resveratrol and 16.67 grams of quercetin and stir until fully incorporated. Maintain the temperature between 40° C. and 45° C.

Formation of the preliminary emulsion: Once both the oil and aqueous phases have reached the established temperature, the oil phase is slowly and gradually added to the aqueous phase, under constant stirring. The mixture is stirred for 30 minutes to ensure the formation of a homogeneous preliminary emulsion.

Formation of the nanoemulsion: To reduce the droplet size in the preliminary emulsion to a range of 50 to 200 nanometers (nm), a high-energy ultrasonic homogenizer is used. The sonication process is carried out in 10-second sonication cycles followed by 10 seconds of rest, for a total period of 15 to 20 minutes. During the entire sonication process, the temperature of the emulsion is maintained at 45° C.

Cooling of the nanoemulsion: After the sonication process is completed, the resulting nanoemulsion is cooled until it reaches a temperature below 30° C., while stirring at a low speed between 30 and 50 rpm, to ensure uniform droplet distribution during cooling.

Capsule filling: Once the nanoemulsion has reached the appropriate temperature, the filling of the capsules is carried out. The nanoemulsion is introduced into soft gelatin capsules.

### Example 2

Oil phase: A quantity of 13.75 milligrams of medium-chain triglycerides (MCT) is added to a suitable container. Subsequently, 5.0 grams of Sorbitan 80 (Span 80) and 0.1 milligrams of Vitamin E are incorporated. Immediately, 6.25 grams of CBD and 2.5 grams of CBG are added. The mixture is stirred at a speed of 350-700 revolutions per minute (rpm) until all components are fully integrated. The oil phase is then heated to a temperature between 60° C. and 70° C.

Aqueous phase: Heat 46.45 milligrams of purified water to 90° C. and dissolve 0.18 grams of methylparaben and 0.02 grams of propylparaben. Cool the solution to a temperature between 60° C. and 70° C. Add 2.0 grams of polyethylene glycol 400 (PEG 400) and 5.0 grams of polysorbate 80 (Tween 80), and stir until all ingredients are completely incorporated. Add 6.25 grams of resveratrol and 12.5 g of quercetin and continue stirring until all components are fully integrated. Maintain the temperature between 60° C. and 70° C.

Formation of the preliminary emulsion: Once both the oil and aqueous phases have reached the established temperature, the oil phase is slowly and gradually added to the aqueous phase, under constant stirring. The mixture is stirred for 30 minutes to ensure the formation of a homogeneous preliminary emulsion.

Formation of the nanoemulsion: To reduce the droplet size in the preliminary emulsion to a range of 50 to 200 nanometers (nm), a high-pressure homogenizer is used. The homogenization process is carried out by applying pressures between 1000 and 1500 bar, over 3 to 5 cycles of homogenization. During each cycle, the emulsion is forced through a narrow valve at high speed, generating shear forces that reduce the droplet size. It is crucial to monitor the temperature of the emulsion throughout the process, keeping it between 60° C. and 70° C., using a cooling system if necessary, to preserve the stability of the active ingredients.

Cooling of the nanoemulsion: After the sonication process is completed, the resulting nanoemulsion is cooled until it reaches a temperature below 30° C., while stirring at a low speed between 30 and 50 rpm, to ensure uniform droplet distribution during cooling.

Capsule filling: Once the nanoemulsion has reached the appropriate temperature, the filling of the capsules is carried out. The nanoemulsion is introduced into vegetarian capsules.

### Cream Procedure:

### Example 3

Oil phase: A quantity of 10.0 grams of medium-chain triglycerides (MCT) is added to a suitable container. Subsequently, 3.0 grams of cetyl alcohol is incorporated and heated at 60-65° C. until melting. Subsequently, 0.1 grams of Vitamin E, 2.27 grams of CBD, 1.51 grams of CBG, and 2.0 grams of dimethicone are incorporated. The mixture is stirred at a speed of 350-700 revolutions per minute (rpm) until all components are fully integrated.

Aqueous phase: A quantity of 6 grams of alcohol is added to a suitable container. Subsequently, 0.23 grams of capsaicin is incorporated and is stirred at a speed of 500 revolutions per minute (rpm) until all components are fully integrated. The solution is then reserved for further use. Heat 41.29 grams of purified water to 60-65° C. Add 0.1 grams of potassium sorbate, 3 grams of glycerine, and the solution reserved for capsaicin stir until all ingredients are completely incorporated. Add slowly 0.5 grams of hydroxyethyl cellulose and stir with a homogenizer.

Formation of the emulsion: Once both the oil and aqueous phases have reached the established temperature, the oil phase is slowly and gradually added to the aqueous phase, under constant stirring. The mixture is stirred for 30 minutes to ensure the formation of a homogeneous preliminary emulsion.

Cooling of the emulsion: After the emulsion process is completed, the result is cooled until it reaches a temperature below 30° C., while stirring at a low speed between 30 and 50 rpm, to ensure uniform droplet distribution during cooling.

### Example 4

Oil phase: A quantity of 15.0 grams of medium-chain triglycerides (MCT) is added to a suitable container. Subsequently, 5.0 grams of cetyl alcohol is incorporated and heated at 60-65° C. until melting. Subsequently, 0.1 grams of Vitamin E, 2.27 grams of CBD, 1.51 grams of CBG, and 5.0 grams of dimethicone are incorporated. The mixture is stirred at a speed of 350-700 revolutions per minute (rpm) until all components are fully integrated.

Aqueous phase: A quantity of 3 grams of alcohol is added to a suitable container. Subsequently, 0.23 grams of capsaicin is incorporated and is stirred at a speed of 500 revolutions per minute (rpm) until all components are fully integrated. The solution is then reserved for further use. Heat 31.69 grams of purified water to 60-65° C. Add 0.2 grams of potassium sorbate, 5 grams of glycerine, and the solution reserved for capsaicin stir until all ingredients are completely incorporated. Add slowly 1.0 grams of hydroxyethyl cellulose and stir with a homogenizer.

Formation of the emulsion: Once both the oil and aqueous phases have reached the established temperature, the oil phase is slowly and gradually added to the aqueous phase, under constant stirring. The mixture is stirred for 30 minutes to ensure the formation of a homogeneous preliminary emulsion.

Cooling of the emulsion: After the emulsion process is completed, the result is cooled until it reaches a temperature below 30° C., while stirring at a low speed between 30 and 50 rpm, to ensure uniform droplet distribution during cooling.

1. A composition for the treatment of shingles symptoms, comprising:
a) at least one cannabinoid;
b) a lipid agent;
c) optionally, one or more additional active agents including polyphenols, flavonoids, alkaloids, or aloe vera;
wherein the composition is suitable for oral administration in a capsule form or topical administration in a cream form.

2. The composition according to claim 1, wherein the cannabinoid comprises cannabidiol (CBD), cannabigerol (CBG) or combinations thereof.

3. The composition according to claim 1, wherein the composition is formulated as an oral capsule comprising:
a) at least two cannabinoids;
b) at least one polyphenol and one flavonoid;
c) a stabilizing solubilizing agent.

4. The composition according to claim 1, wherein the composition is formulated as a topical cream comprising:
a) at least two cannabinoids;
b) at least one alkaloid, capsaicin, and aloe vera;
c) an emollient agent.

5. The composition according to claim 3, wherein the cannabinoids in the oral capsule are present in the following concentrations:
a) CBD ranging from 6.0% to 9.0%;
b) CBG ranging from 2.0% to 4.0%;
c) stabilizing solubilizing agents ranging from 12% to 16%.

6. The composition according to claim 4, wherein the cannabinoids in the topical cream are present in the following concentrations:
a) CBD ranging from 2.0% to 3.0%;
b) CBG ranging from 1.0% to 2.0%.

7. The composition according to claim 3, wherein the stabilizing solubilizing agents are selected from Polysorbate 80, Sorbitan 80, PEG 400, or combinations thereof.

8. A method for treating shingles symptoms in a human individual, comprising: administering an effective concentration of the composition of claim 1, wherein the active principle comprises a combination of cannabinoids, polyphenols, and stabilizing solubilizing agents.

9. The method according to claim 8, wherein the human individual is selected based on a diagnosis of shingles (Herpes Zoster).

10. The method according to claim 8, wherein the composition is administered as an oral capsule to treat systemic symptoms of shingles.

11. The method according to claim 8, wherein the composition is administered as a topical cream to treat localized symptoms such as rash and pain at the site of herpetic lesions.

12. The method according to claim 9, further comprising identifying individuals using medical history, physical examination, blood tests, and other diagnostic methods.

13. The method according to claim 8, wherein the effective concentration is administered as a single capsule containing 220 mg of active ingredients daily for a period of 4 weeks.

14. The method according to claim 8, wherein the effective concentration is administered as a topical cream applied 4 times daily for a period of 4 weeks, containing 34.01 g per 100 g of active ingredients.

15. The composition according to claim 4, wherein the emollient agents include Glycerin and Dimethicone.

16. The method according to claim 8, wherein the administration of the composition provides monotherapy for treating shingles (Herpes Zoster).

17. The method according to claim 8, wherein the composition alleviates symptoms including pain, burning sensation, rash, swelling, itching, fatigue, fever, headache, and sensitivity to touch.

18. A method of producing a pharmaceutical composition for the treatment of shingles, comprising:
a) incorporating cannabinoids and isoflavones into a nanoplatform formulation;
b) combining the formulation with a stabilizing solubilizing agent;
c) applying nano emulsification techniques to achieve a nanoplatform size of under 200 nm;
d) producing the composition for oral delivery.

19. A kit for the treatment of shingles, comprising:
a) an oral composition in capsule form, the composition comprising at least two cannabinoids, one polyphenol, one flavonoid, and a stabilizing solubilizing agent;
b) a topical cream composition, the composition comprising at least two cannabinoids, one alkaloid, aloe vera, and an emollient agent;
wherein the kit is designed to provide both systemic and localized relief of shingles symptoms.

20. The kit according to claim 19, further comprising instructions for the administration of the oral composition and topical cream, wherein the oral composition is administered as a daily capsule for systemic treatment and the topical cream is applied 4 times daily for localized symptom relief, for a duration of 4 weeks.
